SEQUENCE LISTING (6 sequences)

SEQ ID NO: 1 - DNA, 1239 bp, Homo sapiens
atgggcagcccctggaacggcagcgacggccccgagggggcgcgggagccgccgtggcccgcgctgccgccttgcgacgagcgccgctgctcgccctttcccctgggggcgctggtgccggtgaccgctgtgtgcctgtgcctgttcgtcgtcggggtgagcggcaacgtggtgaccgtgatgctgatcgggcgctaccgggacatgcggaccaccaccaacttgtacctgggcagcatggccgtgtccgacctactcatcctgctcgggctgccgttcgacctgtaccgcctctggcgctcgcggccctgggtgttcgggccgctgctctgccgcctgtccctctacgtgggcgagggctgcacctacgccacgctgctgcacatgaccgcgctcagcgtcgagcgctacctggccatctgccgcccgctccgcgcccgcgtcttggtcacccggcgccgcgtccgcgcgctcatcgctgtgctctgggccgtggcgctgctctctgccggtcccttcttgttcctggtgggcgtcgagcaggaccccggcatctccgtagtcccgggcctcaatggcaccgcgcggatcgcctcctcgcctctcgcctcgtcgccgcctctctggctctcgcgggcgccaccgccgtccccgccgtcggggcccgagaccgcggaggccgcggcgctgttcagccgcgaatgccggccgagccccgcgcagctgggcgcgctgcgtgtcatgctgtgggtcaccaccgcctacttcttcctgccctttctgtgcctcagcatcctctacgggctcatcgggcgggagctgtggagcagccggcggccgctgcgaggcccggccgcctcggggcgggagagaggccaccggcagaccgtccgcgtcctgctggtggtggttctggcatttataatttgctggttgcccttccacgttggcagaatcatttacataaacacggaagattcgcggatgatgtacttctctcagtactttaacatcgtcgctctgcaacttttctatctgagcgcatctatcaacccaatcctctacaacctcatttcaaagaagtacagagcggcggcctttaaactgctgctcgcaaggaagtccaggccgagaggcttccacagaagcagggacactgcgggggaagttgcaggggacactggaggagacacggtgggctacaccgagacaagcgctaacgtgaagacgatgggataa

SEQ ID NO: 2 - protein, 412 aa, Homo Sapiens
MGSPWNGSDGPEGAREPPWPALPPCDERRCSPFPLGALVPVTAVCLCLFVVGVSGNVVTVMLIGRYRDMRTTTNLYLGSMAVSDLLILLGLPFDLYRLWRSRPWVFGPLLCRLSLYVGEGCTYATLLHMTALSVERYLAICRPLRARVLVTRRRVRALIAVLWAVALLSAGPFLFLVGVEQDPGISVVPGLNGTARIASSPLASSPPLWLSRAPPPSPPSGPETAEAAALFSRECRPSPAQLGALRVMLWVTTAYFFLPFLCLSILYGLIGRELWSSRRPLRGPAASGRERGHRQTVRVLLVVVLAFIICWLPFHVGRIIYINTEDSRMMYFSQYFNIVALQLFYLSASINPILYNLISKKYRAAAFKLLLARKSRPRGFHRSRDTAGEVAGDTGGDTVGYTETSANVKTMG

SEQ ID NO: 3 - protein, 17 aa, Homo Sapiens
REPPWPALPPCDERRCS

SEQ ID NO: 4 - protein, 19 aa, Homo Sapiens
SPPSGPETAEAAALFSREC

SEQ ID NO: 5 - protein, 20 aa, Homo Sapiens
SSRRPLRGPAASGRERGHRQ

SEQ ID NO: 6 - protein, 16 aa, Homo Sapiens
RKSRPRGFHRSRDTAG

(19) United States
(12) Patent Application Publication
(10) Pub. No.: US 2003/0186336 A1
(43) Pub. Date: Oct. 2, 2003

(54) Diagnostic and therapeutic compositions and methods related to GPCR 38, a G protein-coupled receptor (GPCR)

(76) Inventors: Glenna C. Burmer, Seattle, WA (US); Bruce G. Kulander, Seattle, WA (US); Christine L. Roush, Seattle, WA (US); Joseph P. Brown, Seattle, WA (US)

Correspondence Address: Joshua King, GRAYBEAL JACKSON HALEY LLP, Suite 350, 155-108th Avenue N.E., Bellevue, WA, 98004-5901, US

(21) Appl. No.: 10/206,677

(22) Filed: Jul. 26, 2002

(30) Foreign Application Priority Data
Nov. 29, 2001 (WO) PCT/US01/45219

(51) Int. Cl. G01N 33/567

(52) U.S. Cl. 435/7.21

(57) ABSTRACT

The present invention comprises systems, methods, compositions and the like, such as diagnostics, medicaments and therapeutics, relating to GPR 38 and Alzheimer's disease and Parkinson's disease, inflammatory bowel diseases including ulcerative colitis and Crohn's disease, Hodgkin's disease, glioblastoma and carcinomas including breast, colon, lung (small cell and adenocarcinoma) pancreatic (small cell and adenocarcinoma), ovarian, and prostate. Such diagnostics and therapeutics include peptide, protein, antibody and nucleic acid based compositions, including agonists, antagonists, probes, antisense and gene therapy compositions.

### CROSS-REFERENCE TO RELATED APPLICATIONS

The present application claims priority from PCT patent application PCT/US01/45219, filed Nov. 29, 2001, which application claims priority from U.S. provisional patent application Ser. No. 60/250,251, filed Nov. 29, 2000, and U.S. provisional patent application Ser. No. 60/250,452, filed Nov. 30, 2000, both of which are presently pending.

### TABLE OF CONTENTS

The following is a Table of Contents to assist review of the present application:

CROSS-REFERENCE TO RELATED APPLICATIONS

TABLE OF CONTENTS

BACKGROUND

SUMMARY

BRIEF DESCRIPTION OF THE DRAWING

DETAILED DESCRIPTION

A. INTRODUCTION AND OVERVIEW

B. DEFINITIONS

C. GENERAL DISCUSSION OF NUCLEIC ACIDS AND POLYPEPTIDES RELATED TO GPR 38

EXPRESSION PROFILE OF GPR 38:

1. DISCUSSION DIRECTED GENERALLY TO NUCLEIC ACIDS

NUCLEIC ACIDS GENERALLY:

ANALOGS/VARIANTS:

RELATED GENES:

NUCLEIC ACIDS DEFINED BY HYBRIDIZATION:

PROBES:

GPCR POLYNUCLEOTIDES IN VECTORS:

EXPRESSION PROFILE BASED ON mRNA:

2. DISCUSSION DIRECTED GENERALLY TO POLYPEPTIDES

POLYPEPTIDES GENERALLY:

EXPRESSION PROFILES BASED ON PROTEINS:

SCREENING FOR GPCR ACTIVITY:

PROTEIN PURIFICATION:

D. CERTAIN ASSAYS, ANTIBODIES, PROBES, THERAPEUTICS, AND OTHER SYSTEMS AND ASPECTS, OF THE INVENTION

1. IDENTIFYING BINDING AGENTS AND MODULATING AGENTS

METHODS FOR IDENTIFYING BINDING AGENTS AND MODULATING AGENTS:

BIOLOGICAL ACTIVITY ASSAY:

SUPPLYING BIOLOGICAL ACTIVITY OR FUNCTIONALITY OF THE GPCR:

USING BIOLOGICAL ACTIVITY OR FUNCTIONALITY OF THE GPCR:

2. SYSTEMS AND METHODS FOR SCREENING FOR AGONISTS AND ANTAGONISTS

a. Generally

SCREENING FOR AGONISTS AND ANTAGONISTS—PURPOSES OF SAME:

CONSTITUTIVELY ACTIVE RECEPTOR FOR SCREENING FOR ANTAGONISTS:

SCREENING FOR RECEPTOR DIVERSITY:

b. Labeled Ligand Assays

LABELED LIGAND ASSAYS:

LABELED LIGAND ASSAY—BASIC BINDING ASSAY:

c. Functional Assays for Antagonists or Agonists of GPR 38 FUNCTIONAL ASSAYS:

FUNCTIONAL ASSAYS—MEASURING ANTAGONIST ACTIVITY:

3. SYSTEMS AND METHODS FOR SCREENING FOR GPR 38

POLYPEPTIDE OR POLYNUCLEOTIDE

SCREENING FOR POLYPEPTIDE OR POLYNUCLEOTIDE:

a. Assays Based On GPR 38 Polypeptides

SCREENING FOR/WITH POLYPEPTIDE:

LIST OF ASSAYS:

ENZYME-LINKED IMMUNOSORBENT ASSAYS (ELISA):

IMMUNOFLUORESCENCE ASSAY:

BEAD AGGLUTINATION ASSAYS:

ENZYME IMMUNOASSAYS:

SANDWICH ASSAY:

SEQUENTIAL AND SIMULTANEOUS ASSAYS:

IMMUNOSTICK (DIP-STICK) ASSAYS:

IMMUNOCHROMATOGRAPHIC ASSAYS:

IMMUNOFILTRATION ASSAYS:

BIOSENSOR ASSAYS:

b. Assays Based On GPR 38 Polynucleotides

SCREENING FOR/WITH POLYNUCLEOTIDES—PROBES:

4. ANTIBODIES

ANTIBODIES GENERATED AGAINST GPR 38:

ANTIBODIES GENERALLY:

ANTI-IDIOTYPIC ANTIBODIES:

a. Antibody Preparation

(i) Polyclonal Antibodies

ANTIBODY PREP—POLYCLONAL:

ANTIBODY PREP—ADJUVANTS (ALL ABS):

(ii) Monoclonal Antibodies

ANTIBODY PREP—MONOCLONAL:

MOABS—COMBINATORIAL:

HUMANIZED MOAB:

ANTIBODY SUBSTITUTIONS—NON-IMMUNOGLOBULIN POLYPEPTIDES

(ALL ABS):

CHIMERICS:

ANTIBODY LABELING (ALL ABS):

(iii) Humanized And Human Antibodies

HUMANIZED AB GENERALLY:

(iv) Antibody Fragments

ANTIBODY FRAGMENTS:

(v) Bispecific Antibodies

BISPECIFIC ANTIBODIES GENERALLY:

ANTIBODIES—HYBRID IMMUNOGLOBULIN HEAVY CHAIN:

ANTIBODIES—CROSS-LINKED OR “HETEROCONJUGATE”:

ANTIBODIES—DIABODIES:

ANTIBODIES—OTHER:

b. Antibody Purification

ANTIBODY PURIFICATION GENERALLY:

BEFORE LPHIC:

LPHIC:

POST LPHIC:

c. Some Uses For Antibodies Described Herein

(i) Generally

GENERALLY:

ASSAYS:

DIAGNOSTIC USES:

(ii) Assays

ASSAYS:

COMPETITIVE BINDING ASSAYS:

(iii) Affinity Purification

AFFINITY PURIFICATION:

(iv) Therapeutics

THERAPEUTIC USES:

THERAPEUTIC FORMULATIONS:

THERAPEUTIC FORMULATIONS—STERILE:

THERAPEUTIC ADMINISTRATIONS:

THERAPEUTIC ADMINISTRATIONS—SUSTAINED RELEASE-POLYMERS:

THERAPEUTIC ADMINISTRATIONS—SUSTAINED RELEASE-LIPOSOMES:

THERAPEUTICALLY EFFECTIVE AMOUNT:

5. DRUG DESIGN BASED ON GPR 38

USE OF GPR 38 FOR DRUG DESIGN:

6. THERAPEUTICS RELATED TO GPR 38

a. Generally

COMPOSITIONS—CARRIERS, ADJUVANTS, ETC.:

SUSTAINED RELEASE:

THERAPEUTIC APPLICATIONS:

MODULATING BIOLOGICAL ACTIVITY:

ROUTES OF ADMINISTRATION:

b. Discussion Directed Primarily To Polypeptides

DOSAGE REGIMENS:

ADMINISTRATION PROTOCOLS:

PHARMACEUTICAL ADDITIVES (CARRIERS, ADJUVANTS, BUFFERING AGENTS, DISPERSING AGENTS):

c. Discussion Directed Primarily To Polynucleotides

GENERAL:

CARRIERS AND DILUENTS:

ANTISENSE:

RIBOZYMES:

GENE THERAPY:

VECTORS GENERALLY—GDV:

GDV—VIRAL VECTORS:

GDV—NAKED VECTORS:

GDV—LIPOSOMES:

GDV—BACTERIAL CELLS:

GDV—EVENT SPECIFIC PROMOTERS:

GDV—TISSUE SPECIFIC PROMOTERS:

GDV—TISSUE AND EVENT SPECIFIC PROMOTERS:

GDV—OTHER SPECIFIC CONTROL ELEMENTS:

GDV—CANCER DIRECTED VECTOR SYSTEMS:

GDV—EUKARYOTIC LAYERED SYSTEMS:

EXAMPLES

EXAMPLE 1: SELECTION OF ANTIGENS

EXAMPLE 2: ANTIBODY PRODUCTION SCHEDULE

EXAMPLE 3: IMMUNOSORBENT PURIFICATION OF ANTISERUM:

COUPLING OF PEPTIDE TO CNBR-ACTIVATED SEPHAROSE 4B

EXAMPLE 4: IMMUNOSORBENT PURIFICATION OF ANTISERUM:

AFFINITY PURIFICATION OF ANTISERUM

EXAMPLE 5: PREPARATION OF ANTIBODY DILUTIONS

EXAMPLE 6: PREPARATION OF AUTOSTAINER SOLUTIONS

EXAMPLE 7: PREPARATION OF SOLUTIONS FOR ANTIBODY

DETECTION

EXAMPLE 8: DEPARAFFINIZATION AND REHYDRATION OF

SAMPLES

EXAMPLE 9: STEAM METHOD OF TARGET RETRIEVAL

EXAMPLE 10: ANTIBODY DETECTION

EXAMPLE 11: WESTERN BLOTTING

EXAMPLE 12: RESULTS

SEQUENCE LISTING

CLAIMS

ABSTRACT

### BACKGROUND

G protein-coupled receptors (GPCRs) are a large group of proteins that transmit signals across cell membranes. In general terms, GPCRs function somewhat like doorbells. When a molecule outside the cell contacts the GPCR (pushes the doorbell), the GPCR changes its shape and activates “G proteins” inside the cell (similar to the doorbell causing the bell to ring inside the house, which in turn causes people inside to answer the door). In addition, GPCRs are like high-security doorbells because each GPCR responds to only one specific kind of signaling molecule (called its “endogenous ligand”). Part of the GPCR is located outside the cell (the “extracellular domain”), part spans the cell's membrane (the “transmembrane domain”), and part is located inside the cell (the “intracellular domain”). GPCRs are embedded in the outer membrane of a cell and recognize and bind certain types of signaling molecules that are present in the spaces surrounding the cell. GPCRs are used by cells to keep an eye on the cells' own activity and environment. In organisms having many cells, the cells use GPCRs to talk to each other.

GPCRs are of great interest to the pharmaceutical industry and other industries. For example, many drugs act by binding to specific GPCRs and initiating their intracellular actions, and diagnostics and therapeutics based on GPCRs are becoming increasingly important. Databases, such as LifeSpan BioScience's GPCR Database, help researchers to compare and contrast different GPCRs so that various GPCR functions can be investigated and established. With greater knowledge about the distribution of GPCRs in human tissues and their involvement in disease processes, researchers can design more diagnostics and more effective drugs with fewer side effects.

General concepts about GPCRs in general and the GPCR known as GPR 38 in particular are discussed in more scientific terms in the following paragraphs.

The GPCR superfamily has at least 250 members, Strader et al., FASEB J., 9:745-754 (1995); Strader et al., Annu. Rev. Biochem., 63:101-32 (1994). GPCRs play important roles in diverse cellular processes including cell proliferation and differentiation, leukocyte migration in response to inflammation, gene transcription, vision (the rhodopsins), smell (the olfactory receptors), neurotransmission (muscarinic acetylcholine, dopamine, and adrenergic receptors), and hormonal response (luteinizing hormone and thyroid-stimulating hormone receptors). Strader et al., supra; U.S. Pat. Nos. 5,994,097 and 6,063,596. Many important drugs produce their therapeutic actions through their interaction with GPCRs.

GPR 38 is also known as G protein-coupled receptor 38, motilin receptor, and MTLR1, and, in the database system maintained at LifeSpan Biosciences, Seattle Washington, as LSID 160055. GPR 38 is found at chromosome 13, cytoband q 14.21. GPR 38 has been reported to be expressed in enteric neurons, and the human duodenum and colon, Feighner, S. D., et al., Science 284(5423):2184-2188 (1999) (PMID: 10381885), in the stomach, thyroid gland, and bone marrow (human), McKee K. K., et al., Genomics 46(3):426-434 (1997) (PMID: 9441746, and brain, Depoortere I., et al., Brain Res. 777(1-2):103-109 (1997) (PMID: 9449418). GPR 38 is reportedly a heterotrimeric guanosine triphosphate-binding protein (G)-coupled receptor. Feighner, supra.

A DNA sequence for GPR 38 is known and can be found at accession number NM001507; a prototype protein sequence can be found at accession number NP001498.1 SEQ ID NO: 1 and SEQ ID NO: 2, respectively; FIG. 1. See WO9964436. Sequences for GPR 38 can also be found in GenBank at AF034632, AX154589, and AX154591, and in GenPept, SwissProt at AAC26081.1, CAC19107.1, CAC42611.1, CAC42612.1, and O43193. It has been reported that one EST (BF513101) aligns to NM001507 from about 835 to about 901 (about 67 residues out of about 590 nucleotides).

Generally speaking, different GPCRs show both structural and sequence similarities. The most conserved domains of GPCRs are the transmembrane domains and the first two cytoplasmic loops. GPCRs range in size from under 400 to over 1000 amino acids. Coughlin, S. R., Curr. Opin. Cell Biol. 6:191-197 (1994). They contain seven hydrophobic transmembrane regions that span the cellular membrane and form a bundle of antiparallel alpha helices, and GPR 38 appears to be typical in this regard. McKee K. K., supra. The bundle of helices forming the transmembrane regions provide many structural and functional features of the receptor. In most cases, the bundle of helices form a pocket that binds a signaling molecule. However, when the binding site accommodates larger molecules, the extracellular N-terminal segment or one or more of the three extracellular loops participate in binding and in subsequent induction of conformational change in the intracellular portions of the receptor. These helices are joined at their ends by three intracellular and three extracellular loops. GPCRs also contain cysteine disulfide bridges between the second and third extracellular loops, an extracellular N-terminus, and a cytoplasmic or intracellular C-terminus. The N-terminus is often glycosylated, while the C-terminus is generally phosphorylated. A conserved, acidic-Arg-aromatic triplet present in the second cytoplasmic loop may interact with G Proteins. Most GPCRs contain a characteristic consensus pattern. Watson, S. and S. Arkinstall, The G protein Linked Receptor Facts Book, Academic Press, San Diego, Calif. (1994); Bolander, F. F. Molecular Endocrinology, Academic Press, San Diego, Calif. (1994).

GPCRs bind a diverse array of specific, extracellular signaling molecules (which can also be referred to as “ligands”) including peptides, cytokines, hormones, neurotransmitters, growth factors, and specialized stimuli such as photons, flavorants, and odorants. Identified ligands include, for example, purines, nucleotides (e.g., adenosine, cAMP, NTPs), biogenic amines (e.g., epinephrine, norepinepherine, dopamine, histamine, noradrenaline, serotonin), acetylcholine, peptides (e.g., angiotensin, calcitonin, chemokines, corticotropin releasing factor, galanin, growth hormone releasing hormone, gastric inhibitory peptide, glucagon, neuropeptide Y, neurotensin, opioids, thrombin, secretin, somatostatin, thyrotropin releasing hormone, vasopressin, vasoactive intestinal peptide), lipids and lipid-based compounds (e.g., cannabinoids, platelet activating factor), excitatory and inhibitory amino acids (e.g., glutamate, GABA), ions (e.g., calcium), and toxins.

In general, a GPCR binds only one type of signaling molecule and GPCRs are classified according to subfamilies based upon their selectivity and specificity for a particular ligand. When the ligand for a receptor is not known, the receptor is known as an orphan receptor. The extracellular domain interacts with or binds to certain signaling molecules or ligands located outside of the cell. The binding of a ligand to the extracellular domain alters the conformation of the receptor's intracellular domain causing the activation of a G protein. The G protein then activates or inactivates a separate plasma-membrane-bound enzyme or ion channel. This chain of events alters the concentration of one or more intracellular messengers (second messengers) such as cyclic AMP (cAMP), inositol triphosphate, diacylglycerol, or Ca²⁺. These, in turn, alter the activity of other intracellular proteins such as cAMP-dependent protein kinase and Ca²⁺/calmodulin-dependent protein kinases, leading to the transduction and amplification of the original extracellular signal. Baldwin, J. M., Curr. Opin. Cell Biol. 6:180-190 (1994). The G protein is deactivated by hydrolysis of GTP by GTPase. U.S. Pat. Nos. 5,994,097 and 6,063,596.

GPCR mutations, both of the loss-of-function and of the activating variety, have been associated with numerous human diseases, Coughlin, supra. For example, retinitis pigmentosa may arise from either loss-of-function or activating mutations in the rhodopsin gene. Somatic activating mutations in the thyrotropin receptor cause hyperfunctioning thyroid adenomas, Parma, J. et al., Nature 365:649-651 (1993). Parma et al. indicate that it may be possible that certain G protein-coupled receptors susceptible to constitutive activation may behave as proto-oncogenes. Interestingly, GPCRs have functional homologues in human cytomegalovirus and herpesvirus, so GPCRs may have been acquired during evolution for viral pathogenesis, Strader et al., FASEB J., 9:745-754 (1995); Arvanitakis et al., Nature, 385:347-350 (1997); Murphy, Annu. Rev. Immunol. 12:593-633 (1994). The importance of the GPCR superfamily is further highlighted by the recent discoveries that some of its family members, the chemokine receptors CXCR4/Fusin and CCR5, are co-receptors for T cell-tropic and macrophage-tropic HIV virus strains, respectively, Alkhatib et al., Science, 272:1955 (1996); Choe et al., Cell, 85:1135 (1996); Deng et al., Nature, 381:661 (1996); Doranz et al., Cell, 85:1149 (1996); Dragic et al., Nature, 381:667 (1996); Feng et al., Science, 272:872 (1996). It is conceivable that blocking these receptors may prevent infection by the human immunodeficiency (HIV) virus. Other GPCR-related items include regulating cellular metabolism and diagnosing, treating and preventing particular diseases associated with particular GPCRs.

One important way to evaluate GPCRs as novel drug targets and for other purposes is through the creation and use of databases. Such databases can provide large amounts of information about genes, proteins, and other biological matter. An excellent example of such a database is the GPCR database created and maintained by LifeSpan BioSciences, Inc., Seattle, Wash., USA, which database is available by subscription to researchers and others needing such information. The information in the databases can, for example, be searched, compared, and analyzed. The compilation of such databases, as well as the searching, comparing, etc., of the databases, can be referred to as the field of “bioinformatics.” Investigations largely related to genes, such as the information found from the sequencing of the human genome, can be called “genomics” while similar activities on proteins can be called “proteomics.”

Thus, there has gone unmet a need for improved systems, compositions, methods and the like relating to GPR 38, including diagnostics and therapeutics related to the expression or absence of expression of GPR 38 in certain tissues or in relation to certain diseases. The present invention provides these and other advantages.

### SUMMARY

The present invention comprises systems, methods, compositions and the like, such as diagnostics, medicaments, and therapeutics, relating to GPR 38 and Alzheimer's disease and Parkinson's disease, inflammatory bowel diseases including ulcerative colitis and Crohn's disease, Hodgkin's disease, glioblastoma, and carcinomas including breast, colon, lung (small cell and adenocarcinoma), pancreatic (small cell and adenocarcinoma), ovarian, and prostate. GPR 38 is a known gene and known corresponding protein. Representative nucleic acid and amino acid sequences for GPR 38 are provided in FIG. 1 as SEQ ID NO: 1 and SEQ ID NO: 2, respectively. Such diagnostics and therapeutics include peptide, protein, antibody, and nucleic acid based compositions, including agonists, antagonists, probes, antisense, and gene therapy compositions.

Thus, in one aspect the present invention provides assays for the detection of an increased possibility of Alzheimer's disease in a human patient, comprising: a) providing a binding partner specific for GPR 38, b) contacting the binding partner with at least one of neurons and astrocytes of the patient under conditions suitable and for a time sufficient for the binding partner to bind to GPR 38 in the at least one of the neurons and astrocytes, c) detecting the binding partner bound to the GPR 38, and d) determining whether the at least one of the neurons and astrocytes contain reduced levels of GPR 38 relative to normal and therefrom determining whether the patient has an increased possibility of Alzheimer's disease.

In another aspect the present invention provides assays for the detection of an increased possibility of Parkinson's disease in a human patient, comprising: a) providing a binding partner specific for GPR 38, b) contacting the binding partner with at least one of neurons and neuropil from a substantia nigra of the patient under conditions suitable and for a time sufficient for the binding partner to bind to GPR 38 in the at least one of the neurons and neuropil, c) detecting the binding partner bound to the GPR 38, and d) determining whether the at least one of the neurons and neuropil contain decreased levels of GPR 38 relative to normal and therefrom determining whether the patient has an increased possibility of Parkinson's disease.

In a further aspect the present invention provides assays for the detection of an increased possibility of ulcerative colitis in a human patient, comprising: a) providing a binding partner specific for GPR 38, b) contacting the binding partner with at least one of surface epithelium, neuroendocrine cells, and enteric plexus ganglion cells and with at least one of subsets of transformed lymphoid cells and subsets of reactive fibroblasts from a colon of the patient under conditions suitable and for a time sufficient for the binding partner to bind to GPR 38 in the at least one of the surface epithelium, neuroendocrine cells, and enteric plexus ganglion cells and in the at least one of the subsets of transformed lymphoid cells and subsets of reactive fibroblasts, c) detecting the binding partner bound to the GPR 38, and d) determining whether the at least one of the surface epithelium, neuroendocrine cells, and enteric plexus ganglion cells contain reduced levels of GPR 38 relative to normal and determining whether the at least one of the subsets of transformed lymphoid cells and reactive fibroblasts contain increased levels of GPR 38 relative to normal, and therefrom determining whether the patient has an increased possibility of ulcerative colitis.

In other aspects the present invention provides assays for the detection of an increased possibility of Crohn's disease in a human patient, comprising: a) providing a binding partner specific for GPR 38, b) contacting the binding partner with at least one of absorptive epithelium and neuroendocrine cells or with at least one of eosinophils from a small intestine of the patient under conditions suitable and for a time sufficient for the binding partner to bind to GPR 38 in the at least one of the absorptive epithelium and neuroendocrine cells or in the at least one of eosinophils, c) detecting the binding partner bound to the GPR 38, and d) determining whether the at least one of the absorptive epithelium and neuroendocrine cells contain reduced levels of GPR 38 relative to normal and determining whether the at least one of eosinophils contain increased levels of GPR 38 relative to normal, and therefrom determining whether the patient has an increased possibility of Crohn's disease.

The assay can also be for the detection of an increased possibility of Hodgkin's disease in a human patient, comprising: a) providing a binding partner specific for GPR 38, b) contacting the binding partner with Reed Sternberg cells and reactive lymphoid cells from the patient under conditions suitable and for a time sufficient for the binding partner to bind to GPR 38 in the Reed Sternberg cells and reactive lymphoid cells, c) detecting the binding partner bound to the GPR 38, and d) determining whether the Reed Sternberg cells contain increased levels of GPR 38 relative to normal and the reactive lymphoid cells contain focal punctuate staining of GPR 38, and therefrom determining whether the patient has an increased possibility of Hodgkin's disease.

Alternatively, the assay can be for the detection of an increased possibility of glioblastoma in a human patient, comprising: a) providing a binding partner specific for GPR 38, b) contacting the binding partner with neoplastic glial cells from the patient under conditions suitable and for a time sufficient for the binding partner to bind to GPR 38 in the neoplastic glial cells and reactive lymphoid cells, c) detecting the binding partner bound to the GPR 38, and d) determining whether the neoplastic glial cells contain increased levels of GPR 38 relative to normal and therefrom determining whether the patient has an increased possibility of glioblastoma.

In still a further aspect the present invention provides assays for the detection of an increased possibility of carcinoma selected from the group consisting of breast carcinoma, colon carcinoma, lung small cell carcinoma, lung adenocarcinoma, ovarian carcinoma, pancreatic small cell carcinoma, pancreatic adenocarcinoma and prostate carcinoma in a human patient, comprising: a) providing a binding partner specific for GPR 38, b) contacting the binding partner with cells from a tissue selected from the group consisting of breast, colon, lung, ovarian, pancreas and prostate from the patient under conditions suitable and for a time sufficient for the binding partner to bind to GPR 38 in the tissue from the group consisting of breast, colon, lung, ovarian, pancreas and prostate, c) detecting the binding partner bound to the GPR 38, and d) determining whether the tissue from the group consisting of breast, colon, lung, ovarian, pancreas and prostate contain increased levels of GPR 38 relative to normal and therefrom determining whether the patient has an increased possibility of carcinoma selected from the group consisting of breast carcinoma, colon carcinoma, lung small cell carcinoma, lung adenocarcinoma, ovarian carcinoma, pancreatic small cell carcinoma, pancreatic adenocarcinoma and prostate carcinoma wherein the tissue selected corresponds to the tissue potentially containing the possible carcinoma.

In certain embodiments of these assays, the binding partner can be an antibody. The tissues of interest can be in at least one biopsy removed from a living patient or in at least one tissue sample removed from a deceased patient. The present invention provides kits for the detection of antibodies against GPR 38 for use in an assay as described herein. The kit can comprise an antibody specific for GPR 38, one or both of a reagent or a device for detecting the antibody, and a label stating that the kit is to be used in the assay. The label can be an FDA approved label.

The present invention further provides isolated and purified compositions comprising GPR 38 and a pharmaceutically acceptable carrier for use in the manufacture of a medicament for inhibiting, preventing or treating at least one of Alzheimer's disease, Parkinson's disease, ulcerative colitis, Crohn's disease, Hodgkin's disease, glioblastoma, breast carcinoma, colon carcinoma, lung small cell carcinoma, lung adenocarcinoma, pancreatic small cell carcinoma and pancreatic adenocarcinoma and other diseases described herein. The present invention also provides methods of manufacturing a medicament able to reduce symptoms associated with diseases described herein in a human patient, comprising combining a pharmaceutically effective amount of a GPR 38 agonist, a pharmaceutically acceptable carrier, adjuvant, excipient, buffer and diluent. Also provided are methods of manufacturing a medicament able to reduce symptoms associated with diseases described herein in a human patient, comprising combining a pharmaceutically effective amount of a GPR 38 antagonist, a pharmaceutically acceptable carrier, adjuvant, excipient, buffer and diluent

These and other aspects, features, and embodiments are set forth within this application, including the following Detailed Description and attached drawings. The present invention comprises a variety of aspects, features, and embodiments; such multiple aspects, features, and embodiments can be combined and permuted in any desired manner. In addition, various references are set forth herein, including in the Cross-Reference To Related Applications, that discuss certain compositions, apparatus, methods, or other information; all such references are incorporated herein by reference in their entirety and for all their teachings and disclosures, regardless of where the references may appear in this application.

### BRIEF DESCRIPTION OF THE DRAWING

FIG. 1 depicts representative examples of the nucleotide and amino acid sequences of GPR 38.

### DETAILED DESCRIPTION

A. Introduction and Overview

Diseases such as Alzheimer's disease and Parkinson's disease are serious health problems in the modem world. Any improvement in the diagnosis, treatment or other remediation of such diseases is a significant advance for as many as a million or more people. This is also true for inflammatory bowel diseases including ulcerative colitis and Crohn's disease, Hodgkin's disease, glioblastoma, and cancers such as breast cancer, colon cancer, lung cancer (small cell and adenocarcinoma), pancreatic (small cell and adenocarcinoma), ovarian, and prostate cancer. The present invention provides diagnostics, therapeutics, and other helpful compositions based on GPR 38 directed to such diseases and conditions. The compositions, methods, and the like can include one or more of peptide, protein, antibody, nucleic acid, and small molecule components, and can be useful, for example, as agonists, antagonists, probes, antisense, and gene therapy compositions and otherwise as may be desired.

The discussion herein, including the following passages, has been separated by headings for convenience. The disclosure under a given heading is not restricted to that heading. For example, the discussion in the definitions section is a part of the disclosure of the invention, the discussion on polypeptides also contains discussion related to polynucleotides, antibodies, etc., and the discussion on antibodies contains discussion related to therapeutic compositions, etc.

B. Definitions

The following paragraphs provide a non-exhaustive list of definitions of some of the terms and phrases as used herein. All terms used herein, including those specifically described below in this section, are used in accordance with their ordinary meanings unless the context or definition indicates otherwise. Also unless indicated otherwise, except within the claims, the,, use of “or” includes “and” and vice-versa. Non-limiting terms are not to be construed as limiting unless expressly stated (for example, “including” means “including without limitation” unless expressly stated otherwise).

The terms set forth in this application are not to be interpreted in the claims as indicating a “means plus function” relationship unless the word “means” is specifically recited in a claim, and are to be interpreted in the claims as indicating a “means plus function” relationship where the word “means” is specifically recited in a claim. Similarly, the terms set forth in this application are not to be interpreted in method or process claims as indicating a “step plus function” relationship unless the word “step” is specifically recited in the claims, and are to be interpreted in the claims as indicating a “step plus function” relationship where the word “step” is specifically recited in a claim.

“Agonist” indicates a substance, such as a molecule or compound, that interacts with GPR 38, for example by binding to the GPCR, to activate, increase, or prolong the amount or the duration of the effect of the biological activity of the GPCR. Agonists include proteins, nucleic acids, carbohydrates, or any other molecules that bind to and positively modulate the effect of the GPCR. Agonists and other modulators of GPR 38 can be identified using in vitro or in vivo assays for G protein-coupled receptor expression or G protein-mediated signaling. For example, assays for agonists and other modulators include expressing GPR 38 in cells or cell membranes, applying putative modulator compounds in the presence or absence of a specific known ligand (such as motilin) or putative ligand and then determining the functional effects on GPR 38-mediated signaling. Samples or assays comprising GPR 38 that are treated with a potential agonist or other modulator are compared to control samples without the agonist or other modulator to examine the extent of modulation. Control samples can be assigned a relative GPR 38 activity value of 100%. Agonist activity on GPR 38 is achieved when the G protein-coupled receptor activity value relative to the control is at least about 110%, optionally about 150%, preferably about 200-500%, or about 1000-3000% or higher. Down-modulation (for example by an antagonist) of GPR 38 is achieved when the GPR 38 activity value relative to the control is at most about 90%, typically about 80%, optionally about 50% or about 25-0% of the 100% value.

“Aggregate,” see Complex.

“Allele” or “allelic sequence” indicates an alternative form of the gene encoding the GPCR. Alleles may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to alleles are generally ascribed to natural deletions, additions, or substitutions of nucleotides. Each of these types of changes may occur alone or in combination with the others, one or more times in a given sequence.

“Altered” nucleic acid sequences encoding the GPCR include those sequences with deletions, insertions, or substitutions of different nucleotides, resulting in a polynucleotide encoding the same GPCR or a polypeptide variant with at least one substantial structural or functional characteristic of the GPCR. Included within this definition are polymorphisms that may or may not be readily detectable using a particular oligonucleotide probe against the polynucleotide encoding the GPCR. “Altered” proteins may contain deletions, insertions, or substitutions of amino acid residues that produce a silent change and result in a functionally equivalent GPCR. Deliberate amino acid substitutions may be made on the basis of similarity in polarity, charge, solubility, hydrophobicity, hydrophilicity, or the amphipathic nature of the residues, as long as the biological or immunological activity of the GPCR is retained. For example, negatively charged amino acids may include aspartic acid and glutamic acid, positively charged amino acids may include lysine and arginine, and amino acids with uncharged polar head groups having similar hydrophilicity values may include leucine, isoleucine, and valine; glycine and alanine; asparagine and glutamine; serine and threonine; and, phenylalanine and tyrosine.

“Alternative splicing” refers to different ways of cutting and assembling exons to produce mature mRNAs.

“Amino acid” refers generally to any of a class of organic compounds that contains at least one amino group, —NH2, and one carboxyl group, —COOH. The alpha-amino acids, RCH(NH₂)COOH, are the building blocks from which proteins are typically constructed. Amino acid can also refer to artificial chemical analogues or mimetics of a given amino acid as described, depending on the context.

“Amino acid sequence” refers to a string of amino acids, such as an oligopeptide, peptide, polypeptide, or protein sequence, or a fragment of any of these, including naturally occurring or synthetic molecules and those comprising an artificial chemical analogue or mimetic of a given amino acid. In this context, “biologically active fragments,” “biologically functional fragments,” “immunogenic fragments,” and “antigenic fragments” refer to fragments of the GPCR that are preferably about 5 to about 15, 25, or 50 or more amino acids in length and that retain a substantial amount of such activity of the GPCR. Where “amino acid sequence” refers to an amino acid sequence of a naturally occurring protein molecule, “amino acid sequence,” and like terms are not necessarily limited to the complete native amino acid sequence associated with the recited protein molecule.

“Amplification” indicates the production of additional copies of something, such as a nucleic acid sequence. Amplification can be generally carried out using polymerase chain reaction (PCR) technologies or other technologies such as the cycling probe reaction (CPR) that are well known in the art. See, e.g., Dieffenbach, C. W. and G. S. Dveksler, PCR Primer, a Laboratory Manual, pp.1-5, Cold Spring Harbor Press, Plainview, N.Y. (1995); U.S. Pat. Nos. 5,660,988, 5,731,146, and 6,136,533.

“Amplification primers” are oligonucleotides such as natural, analog or artificially created nucleotides that can serve as the basis for the amplification of a selected nucleic acid sequence. They include, for example, both PCR primers and ligase chain reaction oligonucleotides.

“Analog” or “variant” indicates a GPCR that has been modified by deletion, addition, modification, or substitution of one or more amino acid residues in the wild-type receptor. Analogs encompass allelic and polymorphic variants, and also muteins and fusion proteins that comprise all or a significant part of such GPCR, e.g., covalently linked via side-chain group or terminal residue to a different protein, polypeptide, or moiety (fusion partner). Variants of the GPR 38 protein refer to an amino acid sequence that is altered by one or more amino acids, for example by one or more amino acid substitution, insertion, deletion or modification, or proteins with or without associated native-pattern glycosylation. The variant may have “conservative” changes. Such “conservative” changes generally are well known in the art and readily determinable for GPR 38 in view of the present application. Conservative changes include, for example, substitutions where a substituted amino acid has similar structural or chemical properties to the amino acid it replaced (e.g., negatively charged amino acids include aspartic acid and glutamic acid; positively charged amino acids include lysine, arginine, histidine, asparagine, and glutamine; amino acids containing sulfur include methionine and cysteine; polar hydroxy amino acids include serine, threonine, and tyrosine; large hydrophobic amino acids include phenylalanine and tryptophan; small hydrophobic amino acids include alanine, leucine, isoleucine, and valine). A variant may also have “nonconservative” changes which means that the replacement amino acid provides some substantial change in the amino sequence.

A variant preferably retains at least about 80% sequence identity to a native sequence, more preferably at least about 90% identity, and even more preferably at least about 95% identity. Within certain embodiments, such variants contain alterations such that the ability of the variant to bind motilin is not substantially eliminated; in some embodiments the ability to bind motilin is not substantially diminished. Modifications of amino acid residues may include but are not limited to aliphatic esters or amides of the carboxyl terminus or of residues containing carboxyl side chains, 0-acyl derivatives of hydroxyl group-containing residues, and N-acyl derivatives of the amino-terminal amino acid or amino-group containing residues, e.g., lysine or arginine. Guidance in determining which and how many amino acid residues may be substituted, inserted, deleted, or modified without diminishing immunological or biological activity may be found in view of the present application using any of a variety of methods and computer programs known in the art, for example, DNASTAR software. Properties of a variant may generally be evaluated by assaying the reactivity of the variant with, for example, antibodies as described herein or evaluating a biological activity characteristic of the native protein as described herein or as known in the art in view of the present application. Certain polynucleotide variants are capable of hybridizing under appropriately stringent conditions to a naturally occurring DNA sequence encoding GPR 38 protein (or a complementary sequence). Such hybridizing nucleic acid sequences are also within the scope of this invention.

“Antagonist” refers to a molecule which interacts with GPR 38, for example by binding to GPR 38, and prevents, inactivates, decreases, or shortens the amount or the duration of the effect of the biological activity of the GPCR. Antagonists include proteins, nucleic acids, carbohydrates, antibodies, or any other molecules that so affect the GPCR. Antagonists can be identified, for example, using appropriate screens corresponding to those described for agonists above and elsewhere herein or as would be apparent to those skilled in the art in view of the present application.

“Antibody” indicates one type of binding partner, typically encoded by an immunoglobulin gene or immunoglobulin genes, and refers to, for example, intact monoclonal antibodies (including agonist and antagonist antibodies), polyclonal antibodies, phage display antibodies, and multispecific antibodies (e.g., bispecific antibodies) formed, for example, from at least two intact antibodies. Antibody also refers to fragments thereof, which comprise a portion of an intact antibody, generally the antigen-binding or variable region of the intact antibody that are capable of binding the epitopic determinant. Examples of antibody fragments include Fab, Fab′, F(ab′)₂, and Fv fragments, diabodies, linear antibodies, single-chain antibody molecules, and multispecific antibodies formed from antibody fragments. See U.S. Pat. No. 6,214,984. Antibody fragments may be synthesized by digestion of an intact antibody or synthesized de novo either chemically or utilizing recombinant DNA technology. Antibodies according to the present invention have at least one of adequate specificity, affinity, and capacity to perform the activities desired for the antibodies. Antibodies can, for example, be monoclonal, polyclonal, or combinatorial. Antibodies that bind GPCR polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

“Antigenic determinant” refers to the antigen recognition site on an antigen (i.e., epitope). Such antigenic determinant may also be immunogenic. “Antisense” refers to any composition containing a nucleic acid sequence that is complementary to a specific nucleic acid sequence. “Antisense strand” refers to a nucleic acid strand that is complementary to the “sense” strand. Antisense molecules may be produced by any method including transcription or synthesis including synthesis by ligating the gene(s) of interest in a reverse orientation to a desired promoter that permits the synthesis of a complementary strand. Once introduced into a cell, the complementary nucleotides can combine with natural sequences produced by the cell to form duplexes and to block either transcription or translation. The designation “negative” can refer to the antisense strand, and the designation “positive” can refer to the sense strand. “Biologically active,” when referring to a GPCR, indicates that the GPCR retains its receptor site binding of its specific ligand including a mimetic thereof and also transmits signal to activate its native second messenger system.

“Biologically functional,” when referring to a GPCR, indicates a GPCR or a variant, fragment, etc., thereof, that has a functional receptor site able to bind its specific ligand or a mimetic thereof or able to activate its native second messenger system. Such a GPCR may also be biologically active and transmit signal based on such binding to a second messenger such as the GPCR's native second messenger system or another second messenger system such as a marker system, or retain other activity associated with the receptor site. A polypeptide is “biologically functional” if the ability to bind motilin or to activate its native second messenger system is not substantially diminished within a representative in vitro or in vivo assay as described herein, or as would be apparent to those skilled in the art in view of the present application. The term “not substantially diminished” means retaining a functionality that is at least about 90% of the functionality of the native GPCR protein. Appropriate assays designed to evaluate such functionality may be designed based on existing assays known in the art in view of the present application, or on the representative assays provided herein.

“Buffer” refers to a component in a solution to provide a buffered solution that resists changes in pH by the action of its acid-base conjugate components.

“Clone” in molecular biology refers to a vector carrying an insert DNA sequence.

“Cloning” in molecular biology refers to a recombinant DNA technique used to produce multiple, up to millions or more, copies of a DNA sequence. The DNA sequence is inserted into a small carrier or vector (e.g., plasmid, bacteriophage, or virus) and inserted into a host cell for amplification or expression.

“Complementary” or “complementarity” refers to the natural binding of polynucleotides by base pairing. For example, the sequence “A-G-T” binds to the complementary sequence “T-C-A.” Complementarity between two single-stranded molecules may be “partial,” such that only some of the nucleic acids bind, or it may be “complete,” such that all of the nucleotides of at least one of the single-stranded molecules binds to corresponding nucleotides of the other single-stranded molecule. The degree of complementarity between nucleic acid strands has significant effects on the efficiency and strength of the hybridization between the nucleic acid strands. This can be of particular importance in amplification reactions, which can depend upon binding between nucleic acids strands, and in the design and use of peptide nucleic acid (PNA) molecules.

“Complex,” or “aggregate,” indicates a dimer or multimer formed between at least two proteins or other macromolecules, for example a GPCR and its ligand.

“Composition” indicates a combination of multiple substances into an aggregate mixture.

“Composition comprising a given polynucleotide sequence” or “composition comprising a given amino acid sequence” refers broadly to any composition containing the given polynucleotide or amino acid sequence. The composition may comprise a dry formulation, an aqueous solution, or a sterile composition. Compositions comprising polynucleotide sequences encoding the GPCR or fragments of the GPCR may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., SDS), and other components (e.g., Denhardt's solution, dry milk, salmon sperm DNA).

“Consensus sequence” refers to the sequence that reflects the most common choice of base or amino acid at each position from a series of related DNA, RNA, or protein sequences. Areas of particularly good agreement often represent conserved functional domains. The generation of consensus sequences has been subjected to intensive mathematical analysis.

“Conservative changes” to an amino acid sequence, see Analog.

“Constitutively active” refers to GPCRs and their variants that display GPCR receptor activity in the absence of normally required stimulation. Such variants may be identified using the representative in vivo assays for GPCR activity described herein, or as would be apparent to those skilled in the art in view of the present application.

“Constitutively inactive” refers to GPCRs and their variants that fail to display GPCR receptor activity in the presence of normally required stimulation. Such variants may be identified using the representative in vivo assays for GPCR activity described herein, or as would be apparent to those skilled in the art in view of the present application.

“Deletion” refers to a change in the amino acid or nucleotide sequence that results in the absence of one or more amino acid residues or nucleotides.

“Derivative” refers to the chemical modification of the GPCR, of a polynucleotide sequence encoding the GPCR, or of a polynucleotide sequence complementary to a polynucleotide sequence encoding the GPCR. Chemical modifications of a polynucleotide sequence can include, for example, replacement of hydrogen by an alkyl, acyl, or amino group. A derivative polynucleotide encodes a polypeptide that retains at least one biological or immunological function of the natural molecule. A derivative polypeptide can be modified, for example, by glycosylation or pegylation, and retains at least one biological activity or immunological activity of the polypeptide from which it was derived.

“Diabodies” refers to small antibody fragments with two antigen-binding sites, which fragments comprise a heavy-chain variable domain (V_{H}) connected to a light-chain variable domain (V_{L}) on the same polypeptide chain (V_{H}-V_{L}). By using a linker that is too short to allow pairing between the two domains on the same chain, the domains pair with the complementary domains of another chain and create two antigen-binding sites. Diabodies are described, for example, in EP 404,097; WO 93/11161; and Holliger et al., Proc. Natl. Acad. Sci. USA, 90:6444-6448 (1993).

“Digestion” of DNA refers to catalytic cleavage of the DNA with a restriction enzyme that acts only at certain sequences in the DNA. The various restriction enzymes used herein are commercially available and their reaction conditions, cofactors and other requirements were used as would be known to the ordinarily skilled artisan. For analytical purposes, typically 1 μg of plasmid or DNA fragment is used with about 2 units of enzyme in about 20 μl of buffer solution. For the purpose of obtaining DNA fragments for plasmid construction, typically about 5 to 50 μg of DNA are digested with about 20 to 250 units of enzyme in a larger volume. Appropriate buffers and substrate amounts for particular restriction enzymes are specified by the manufacturer. Incubation times of about 1 hour at 37° C. are ordinarily used, but may vary in accordance with the supplier's instructions. After digestion the reaction can be electrophoresed directly on a polyacrylamide gel to obtain the desired fragment.

“Expressed sequence tag” or “EST” refers to a short strand of DNA (typically about 200 base pairs long) which is part of a cDNA. Because an EST is usually unique to a particular cDNA, and because cDNAs correspond to a particular gene in the genome, ESTs can be used to help identify unknown genes and to map their position in the genome. ESTs can also be used to roughly determine the extent to which the protein for a particular gene is expressed in a given tissue.

“Expression vector” is a specialized vector constructed so that the gene inserted in the vector can be expressed in the cytoplasm of a host cell.

“Fragment,” see Portion.

“Gene” refers to the basic unit of heredity that carries the genetic information for a given RNA or protein molecule. A gene is composed of a contiguous stretch of DNA and contains a coding region that is flanked on each end by regions that are transcribed but not translated. A gene is a segment of DNA involved in producing a biologically active or biologically functional polypeptide chain.

“Gene delivery system” indicates a thing such as a system, apparatus or method for the delivery of a nucleic acid of the invention to a target cell or tissue. Examples of gene delivery systems include gene delivery vehicles and gene guns.

“Heterologous” indicates a nucleic acid that comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

“Homology” refers to a degree of complementarity. There may be partial homology or complete homology. The word “identity” may substitute for the word “homology.” A partially complementary sequence that at least partially, and substantially, inhibits a corresponding sequence from hybridizing to a target nucleic acid is referred to as “substantially homologous.” The inhibition of hybridization of the completely complementary sequence to the target sequence may be examined using a hybridization assay (e.g., Southern or Northern blot, in situ hybridization, solution hybridization) under conditions of reduced stringency. A substantially homologous sequence or hybridization probe will compete for and inhibit the binding of a completely homologous sequence to the target sequence under stringency conditions that inhibit non-specific binding but permit specific binding. The absence of non-specific binding may be tested by the use of a second target sequence which lacks even a partial degree of complementarity (e.g., less than about 30% homology or identity). In the absence of non-specific binding, the substantially homologous sequence or probe will not hybridize to the second, non-complementary target sequence.

“Human artificial chromosomes” (HACs) refer to generally linear microchromosomes that may contain DNA sequences of about 6 kb to 10 Mb in size, and which contain the elements required for stable mitotic chromosome segregation and maintenance. Harrington, J. J. et al., Nat. Genet. 15:345-355 1997.

“Humanized antibody” refers to antibody molecules in which the amino acid sequence in the non-antigen-binding regions has been altered so that the antibody more closely resembles a human antibody, and still retains its original binding ability. Typically, humanized antibodies are human immunoglobulins (recipient antibody) in which residues from a complementarity-determining region (CDR) of the recipient are replaced by residues from a CDR of a non-human species (donor antibody) such as mouse, rat or rabbit having the desired specificity, affinity, and capacity. In some instances, Fv framework residues of the human immunoglobulin are replaced by corresponding non-human residues. Furthermore, humanized antibodies may comprise residues that are found neither in the recipient antibody nor in the imported CDR or framework sequences. These modifications are typically made to further refine and optimize antibody performance. In general, the humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin sequence. The humanized antibody optimally also will comprise at least a portion of an immunoglobulin constant region (Fc), typically that of a human immunoglobulin. For further details see, e.g., Jones et al., Nature, 321:522-525 (1986); Reichmann et al., Nature, 332:323-329 (1988); and, Presta, Curr. Op. Struct. Biol., 2:593-596 (1992).

“Hybridization” refers to any process by which a strand of nucleic acids binds with a complementary strand through base pairing.

“Hybridization complex” refers to a complex formed between two nucleic acid sequences by virtue of the formation of hydrogen bonds between complementary bases. A hybridization complex may be formed in solution (e.g., C₀ t or R₀ t analysis) or formed between one nucleic acid sequence present in solution and another nucleic acid sequence immobilized on a solid support (e.g., paper, membranes, filters, chips, pins, or glass slides, polymers, or any other appropriate substrate to which cells or their nucleic acids have been fixed).

“Identity,” see Homology.

“Immunocytochemistry” refers to the use of immunologic methods, including a specific antibody, to study cell constituents.

“Immunohistochemistry” refers to the use of immunologic methods, including a specific antibody, to study specific antigens in tissue slices.

“Immunolocalization” refers to the use of immunologic methods, including a specific antibody, to locate molecules or structures within cells or tissues.

“Immunologically active” refers to the capability of a natural, recombinant, or synthetic GPCR, or any immunogenic fragment thereof, to induce a specific immune response in appropriate animals or cells and to bind with specific antibodies. A polypeptide is “immunologically active” if it is recognized by (e.g., specifically bound by) a B-cell or T-cell surface antigen receptor. Immunological activity may generally be assessed using well known techniques, such as those summarized in Paul, Fundamental Immunology, 3rd ed., 243-247, Raven Press (1993) and references cited therein. Such techniques include screening polypeptides derived from the native polypeptide for the ability to react with antigen-specific antisera or T-cell lines or clones, which may be prepared in view of the present application using well known techniques. Preferably, an immunologically active portion of a GPCR protein reacts with such antisera or T-cells at a level that is not substantially lower than the reactivity of the full-length polypeptide (e.g., in an ELISA or T-cell reactivity assay). Such screens may generally be performed using methods well known to those of ordinary skill in the art in view of the present application, such as those described in Harlow and Lane, Antibodies: A Laboratory Manual, Cold Spring Harbor Press (1988). B-cell and T-cell epitopes may also be predicted via computer analysis.

“Immune response” refers to any of the body's immunologic reactions to an antigen such as antibody formation, cellular immunity, hypersensitivity, or immunological tolerance.

“Insertion” and “addition” when referring to a change in a nucleotide or amino sequence indicate the addition of one or more nucleotides or amino acid residues, respectively, to the sequence.

“In situ hybridization” refers to use of a nucleic acid probe, typically a DNA or RNA probe, to detect the presence of a complementary DNA or RNA sequence in target cells such as cloned bacterial cells, cultured eukaryotic cells, or tissue samples. In situ hybridization can also be used for locating genes on chromosomes. The process can be performed by preparing a microscope slide with cells in metaphase of mitosis, then treating slide with a weak base to denature the DNA. Next, pour radioactively labeled probe onto the slide under hybridizing conditions, expose the slide to a photographic emulsion for a suitable period such as a few days or weeks, then develop the emulsion.

“Isoform” refers to different forms of a protein that may be produced from different genes or from the same gene by alternative RNA splicing.

“Isolated” generally means that the material is removed from its original environment (e.g., the natural environment if it is naturally occurring). When referring to a polynucleotide, isolated means that the polynucletide has been separated from its genome. When referring to protein that is initially expressed as a part of a larger polyprotein, isolated means that the protein has been separated from its polyprotein. Thus, a naturally-occurring polynucleotide or polypeptide present in a living animal is not isolated. But the same polynucleotide or polypeptide, separated from some or all of the coexisting materials in the natural system, is isolated. Such polynucleotides could be part of a vector or such polynucleotides or polypeptides could be part of a composition, and still be isolated provided that such vector or composition is not part of its natural environment.

“Ligand” refers to an ion or molecule that binds with another molecule, such as a GPCR, to form a macromolecule such as a receptor-ligand complex. An “endogenous ligand” refers to a native ligand that binds to the receptor of the GPCR and modulates biological activity or functionality of the GPCR in its native environment. For GPR 38, the endogenous ligand is motilin. A “specific ligand” is a ligand able to bind to the receptor of GPR 38 and modulate the biological activity or functionality of GPR 38; an endogenous ligand is one example of a specific ligand.

“Ligation” refers to the process of forming phosphodiester bonds between two double-stranded nucleic acid fragments. Maniatis, T., J. Sambrook, E. Fritsch, T. Maniatis, Molecular Cloning: A Laboratory Manual, p. 146, Cold Spring Laboratory Press (1989). Unless otherwise provided, ligation may be accomplished using known buffers and conditions with 10 units of T4 DNA ligase (“ligase”) per 0.5 μg of approximately equimolar amounts of the DNA fragments to be ligated.

“Microarray” refers to an array of distinct nucleic acid or amino acid molecules arrayed on a substrate, such as paper, nylon or any other type of membrane, filter, chip, glass slide, or any other suitable solid support. Microarrays can also refer to tissue microarrays, composed of small tissue pieces arranged on a slide. U.S. Pat. No. 5,143,854 and PCT Patent Publication Nos. WO 90/15070 and 92/10092.

“Mimetic” refers to a molecule, e.g., a peptide or non-peptide agent, such as a small molecule, that is able to perform the same biological activity as a certain biologically active agent. For example, some mimetics are molecules comprising the same biological function or activity as GPR 38. The structure of the mimetic can be developed from knowledge of the structure of GPR 38 or portions thereof. For other mimetics, the mimetic is able to effect some or all of the actions of molecules related to GPR 38 such as its endogenous ligand, motilin, or antibodies against GPR 38. Such mimetics can be made, in view of the present application, using techniques well known in the art, see, e.g., U.S. Pat. Nos. 6,197,752; 6,093,697; 6,207,643; 5,849,323, and can be included in the various processes, methods, and systems, etc., described herein, such as databases, binding partner assays, probes, medicaments, and therapeutics.

“Modulate” refers to controllably changing the activity of a substance or other item, such as the biological activity of the GPCR. For example, modulation may cause an increase or a decrease in protein activity, binding characteristics, or other biological, functional, or immunological properties of the GPCR.

“Monoclonal antibody” refers to an antibody obtained from a population of substantially homogeneous antibodies, e.g., the individual antibodies comprising the population are identical except for possible naturally occurring mutations that may be present in minor amounts. Monoclonal antibodies include “chimeric” antibodies (immunoglobulins) in which a portion of the heavy or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. U.S. Pat. No. 4,816,567; Morrison et al., P.N.A.S. USA, 81:6851-6855 (1984). Monoclonal antibodies are highly specific, being directed against a single antigenic site. As a matter of distinction, polyclonal antibody preparations typically include different antibodies directed against different determinants (epitopes) of a target antigen whereas each monoclonal antibody is directed against a single determinant on the antigen. Monoclonal antibodies can be synthesized by hybridoma culture, uncontaminated by other immunoglobulins. For example, the monoclonal antibodies to be used in accordance with the present invention may be made by the hybridoma method first described by Kohler and Milstein, Nature, 256:495 (1975), or may be made by recombinant DNA methods. See, e.g., U.S. Pat. No. 4,816,567. Monoclonal antibodies may also be isolated from phage antibody libraries using the techniques described in Clackson et al., Nature, 352:624-628 (1991), and Marks et al., J. Mol. Biol., 222:581-597 (1991), for example. The modifier “monoclonal” indicates the character of the antibody as being obtained from a substantially homogeneous population of antibodies, and is not to be construed as requiring production of the antibody by any particular method.

“Nonconservative” changes to an amino acid sequence, see Analog.

“Northern blotting” or “Northern analysis” refers to a method used to detect specific RNA sequences. For example, the process can be performed by electrophoresing RNA in a denaturing agarose gel, transferring the gel onto a membrane, and hybridizing with a labeled RNA or DNA probe.

“Nucleic acid sequence” refers to a polymer comprising a string of “nucleic acids” such as an oligonucleotide, or a polynucleotide or fragment thereof. The nucleic acid sequence can be from DNA or RNA of genomic or synthetic origin, may be single-stranded or double-stranded, and may represent the sense or the antisense strand. A nucleic acid sequence can also be a PNA or a DNA-like or RNA-like material. Unless stated otherwise, the term encompasses nucleic acids containing known analogues or mimetics of natural nucleotides that have similar binding properties as the reference nucleic acid.

“Oligonucleotide” refers to a nucleic acid sequence, generally between 6 nucleotides to 60 nucleotides, preferably about 15 to 30 nucleotides, and most preferably about 20 to 25 nucleotides, that can, for example, be used in PCR or other nucleic acid amplification or in a hybridization assay or microarray. “Oligonucleotide” includes “amplimers,” “primers,” “oligomers,” and “probes,” as these terms are commonly defined in the art. Oligonucleotides can be chemically synthesized. Such synthetic oligonucleotides may have no 5′ phosphate and if so will not ligate to another oligonucleotide without adding a phosphate, typically by using an ATP in the presence of a kinase. A synthetic oligonucleotide will ligate to a fragment that has not been dephosphorylated.

“Operably linked” or “operably connected” indicates that one element of an apparatus, system, or method, etc., is connected to another element of the apparatus, system, or method, etc., such that the two elements are able to perform their intended purposes. For example, when a promoter is linked to a polynucleotide to allow transcription of the polynucleotide, it is “operably linked” to the polynucleotide.

“Orphan receptor” refers to a receptor for which the endogenous ligand or other ligands inducing biological activity are not known.

“PCR” or “polymerase chain reaction” refers to an in vitro method that uses oligonucleotide primers, enzymes, and a series of repetitive temperature cycles to generate millions of copies of a nucleic acid, typically DNA, from an original specimen of a specific DNA sequence, which specimen may be present only in a trace amount.

“Peptide nucleic acid” (PNA) refers to a nucleic acid, typically used as an antisense molecule or anti-gene agent, that comprises an oligonucleotide of at least about 5 nucleotides in length linked to a peptide backbone of amino acid residues. The PNA can be soluble, for example by ending in a lysine that confers solubility to the composition. PNAs can bind complementary single-stranded DNA and RNA and stop transcript elongation, and may be pegylated to extend their lifespan in the cell. Nielsen, P. E. et al., Anticancer Drug Des. 8:53-63 (1993).

“Plasmids” refers to extrachromasomal genetic elements composed of DNA or RNA found in both eukaryotic and prokaryotic cells that can propagate themselves autonomously in cells. Plasmids can be used as carriers or vectors to clone DNA molecules. They are designated by a lower case p preceded or followed by capital letters or numbers. The starting plasmids herein are either commercially available, publicly available on an unrestricted basis, or can be constructed from available plasmids in accord with published procedures. In addition, equivalent plasmids to those described are known in the art and will be apparent to the ordinarily skilled artisan in view of the present application.

“Polynucleotide encoding a polypeptide” indicates a polynucleotide that includes only the coding sequence for the polypeptide as well as polynucleotides that include additional coding or non-coding sequence.

“Portion” or “fragment” with regard to a protein (as in “a portion of a given protein”) refers to parts of that protein, a subsequence of the complete amino acid sequence of the receptor containing at least about 8, usually at least about 12, more typically at least about 20, and commonly at least about 30 or more contiguous amino acid residues, up to the entire amino acid sequence minus one amino acid. Thus, a protein “comprising at least a portion of the amino acid sequence of SEQ ID NO: 2” or a protein “comprising at least a portion of the amino acid sequence of GPR 38” encompasses the full-length protein and fragments thereof. A portion or fragment of a nucleic acid refers to nucleic acid sequences that are greater than about 12 nucleotides in length, and typically at least about 60 or 100 nucleotides, generally at least about 1000 nucleotides, or at least about 10,000 nucleotides in length, up to the entire nucleic acid sequence minus one nucleic acid.

“Probe” when referring to nucleic acids indicates a nucleic acid capable of binding to a target nucleic acid (e.g., a nucleic acid encoding GPR 38) that has a complementary sequence via one or more types of chemical bonds, usually through complementary base pairing, usually through hydrogen bond formation. A probe may include natural bases (e.g., A, G, C, or T) or modified bases (e.g., 7-deazaguanosine, inosine). In addition, the bases in a probe may be joined by a linkage other than a phosphodiester bond, so long as it does not prevent hybridization or cause substantial false-positive or false-negative hybridization. Thus, for example, probes may comprise PNA in which the constituent bases are joined by peptide bonds rather than phosphodiester linkages. It will be understood by one of skill in the art in view of the present application that probes may bind target sequences lacking complete complementarity with the probe sequence depending upon the stringency of the hybridization conditions. A “labeled nucleic acid probe” is a nucleic acid probe that is bound, for example via covalent, ionic, van der Waals, or hydrogen bonds, or via a linker, to a label such that the presence of the probe can be determined by detecting the presence of the label bound to the probe.

“Promoter” refers to a nucleotide sequence that contains elements that direct the transcription of a nucleic acid sequence. Generally, a promoter comprises an RNA polymerase binding site. More typically, in eukaryotes, promoter sequences contain binding sites for other transcriptional factors that control the rate and timing of gene expression. Such sites include TATA box, CAAT box, POU box, API binding site, and the like. Promoter regions may also contain enhancer or repressor elements. An “inducible” promoter is a promoter that is active or activatable only under certain, controllable environmental or developmental conditions.

“Receptor” refers to a molecular structure, typically within a cell or on a cell surface, that selectively binds a specific substance (a ligand) and a specific physiologic effect that accompanies the binding. GPCRs are a type of cell-surface receptor, which means a protein in, on, or traversing the cell membrane (in the case of GPCRs, traversing the cell membrane) that recognizes and binds to specific molecules in the surrounding fluid. The binding to a receptor may serve to transport molecules into the cell's interior or to signal the cell to respond in some way.

“Recombinant” refers to both a method of production and a structure. Some recombinant nucleic acids and proteins are made by the use of recombinant DNA techniques that involve human intervention, either in manipulation or selection. Others are made by fusing two fragments that are not naturally contiguous to each other. Engineered vectors are encompassed, as well as nucleic acids comprising sequences derived using any synthetic oligonucleotide process.

“Reverse transcription-polymerase chain reaction” or “rt-PCR” indicates an in vitro method for the analysis of RNA with PCR that first converts RNA into cDNA by reverse transcription using the enzyme reverse transcriptase. The resulting DNA transcript is then amplified by standard PCR methods.

“Sample” is used in its usual broad sense. For example, a biological sample suspected of containing nucleic acids encoding the GPCR, or fragments thereof, or the GPCR itself, may comprise a bodily fluid; an extract from a cell, chromosome, organelle, or membrane from a cell; a cell; genomic DNA, RNA, or cDNA (in solution or bound to a solid support); a tissue; a tissue print, and the like. Biological sample refers to samples from a healthy individual as well as to samples from a subject suspected of having or susceptible to having, e.g., Alzheimer's disease and Parkinson's disease, inflammatory bowel diseases including ulcerative colitis and Crohn's disease, Hodgkin's disease, glioblastoma, and carcinomas including breast, colon, lung (small cell and adenocarcinoma), pancreatic carcinoma (small cell and adenocarcinoma), ovarian carcinoma, and prostate, or any other disease or disorder in which GPR 38 may be involved.

“Second messengers” refer to intracellular signaling molecules such as cyclic AMP (cAMP), inositol triphosphate, diacylglycerol, or Ca²+. It has been reported that a second messenger for GPR 38 is Ca2+because motilin receptor activation leads to increased intracellular Ca2+signaling. Van Assche, G., et al., Neurogastroenterol. Motil. 13:27-35 (2001). Second messengers, in turn, alter the activity of other intracellular proteins such as cAMP-dependent protein kinase and Ca²+/calmodulin-dependent protein kinases, leading to the transduction and amplification of the original extracellular signal.

“Serum albumin” indicates a well known protein found in the blood. Serum albumins are secreted into the blood by liver cells, and bind to and solubilize many small molecules that are only slightly soluble in the blood serum absent such binding by the serum albumin. The folding of the polypeptide chain of serum albumin allows disulfide linkages to form between cysteine residues. The protein contains 3 similar protein domains, and is encoded by a gene having 14 introns and 15 exons. Thus, expression of the gene in eukaryotes and prokaryotes generally includes processing mechanisms or is performed using non-intron containing genes, such as cDNA. Darnell et al., Molecular Cellular Biology, Sci. Am. Books pp. 413-415 (1986); Rothschild et al., N.E.J.M. 286(14):748-757 1972; Sjobring et al., J. Biol. Chem. 266(1):399-405 (1991); Tullis, J., J.A.M.A. 237(4):355-360 (1977); Tullis, J., J.A.M.A. 237(5):460-463 (1977). Serum albumin can be natural, recombinant, purified from an animal source, or produced synthetically.

“Southern blotting” refers to a method for detecting specific DNA sequences via hybridization. For example, a DNA sample can be electrophoresed in a denaturing agarose gel, transferred onto a membrane, and hybridized with a complementary nucleic acid probe.

“Specific binding” or “specifically binding” refers to an interaction between protein or peptide and a certain substance, such as its specific ligand or antibody, and in some cases its agonists or antagonists. The interaction is dependent upon the presence of a particular structure of the protein recognized by the binding molecule (e.g., the antigenic determinant or epitope). For example, if an antibody specifically binds epitope “A,” the presence of a polypeptide containing epitope A or the presence of free unlabeled epitope A will reduce the amount of labeled epitope A that binds to the antibody in a reaction containing free labeled epitope A and the antibody. Conversely, the presence of a polypeptide that does not contain epitope A will not reduce the amount of labeled epitope A that binds to the antibody. Highly specific binding indicates that the protein or peptide binds to its particular ligand, antibody, etc., and does not bind in a significant amount to other proteins present in the sample. Typically, a specific or selective reaction will be at least twice the background signal or noise and more typically more than 10 to 100 times the background signal or noise.

“Stringent conditions” refer to conditions that permit hybridization between complementary polynucleotide sequences. Suitably stringent conditions can be defined by, for example, the concentrations of salt or formamide in the prehybridization and hybridization solutions, or by the hybridization temperature. Stringency can be increased by reducing the concentration of salt, increasing the concentration of formamide, or raising the hybridization temperature. Stringent conditions are dependent upon the type of probe as well as the length of the probe and the GC content of the probe. “Stringent conditions” typically occur within a range from about Tm-5° C. (5° C. below the melting temperature (Tm) of the probe) to about Tm-20-25° C. for a cRNA probe and to about Tm-15° C. for an oligonucleotide probe.

“Highly stringent conditions” refers to conditions under which a probe will hybridize to its target sequence, typically in a complex mixture of nucleic acid sequences, but will not substantially hybridize to other sequences. One example of high stringency conditions for a cRNA probe that is 1,000 nucleotides in length and has a GC content of about 60% is about 55-65° C. in 50% formamide, 0.1×SSC, and 200 μg/ml sheared and denatured salmon sperm DNA. One example of low stringency conditions for the same probe in 50% formamide, 0.1×SSC, and 200 μg/ml sheared and denatured salmon sperm DNA would be 30-35° C. “Very highly stringent conditions” indicates that there must be complete identity between the sequences. The temperature range corresponding to a particular level of stringency can be narrowed further by calculating the purine to pyrimidine ratio of the nucleic acid of interest and adjusting the temperature accordingly. Variations on and modifications of the above ranges and conditions will be readily appreciated by those of skill in the art in view of the present application. As will be understood by those of skill in the art in view of the present application, the stringency of hybridization can be altered to identify or detect identical or related polynucleotide sequences. One guide for nucleic acid hybridization is Tijssen, Laboratory Techniques in Biochemistry and Molecular Biology—v.24 Hybridization with Nucleic Acid Probes, Part I “Overview of principles of hybridization and the strategy of nucleic acid assays” (New York: Elsevier 1993).

“Substantially purified” refers to nucleic acid or amino acid sequences that are removed from their natural environment and are separated from other components from such natural environment, and are at least about 60% free, preferably about 75% or 85% free, and most preferably about 90%, 95% or 99% free from such other components with which they are naturally associated. Substantially purified preferably indicates a substantially homogeneous state and can be in either a dry or aqueous solution or other composition as desired. Purity and homogeneity can be assayed by standard methods, for example on a mass or molar basis, using analytical chemistry techniques such as polyacrylamide gel electrophoresis or high performance liquid chromatography.

“Substitution” when referring to a change in a nucleotide or amino sequence indicates the replacement of one or more nucleotides or amino acids by different nucleotides or amino acids, respectively.

“Transcription terminator region” refers either to a sequence that provides a signal that terminates transcription by the polymerase that recognizes the selected promoter or to a signal sequence for polyadenylation.

“Transformation” and “transfection” refer to a process by which exogenous DNA enters and changes a recipient cell. Transformation may occur under natural or artificial conditions according to various methods well known in the art in view of the present application, and may rely on any known method for the insertion of foreign nucleic acid sequences into the recipient or host cell. The method for transformation is selected based on the type of host cell being transformed and may include, but is not limited to, viral infection, electroporation, heat shock, lipofection, and particle bombardment. “Transformed” cells include stably transformed cells in which the inserted DNA is capable of replication either as an autonomously replicating plasmid or as part of the host chromosome, and refers to cells that transiently express the inserted DNA or RNA for limited periods of time.

“Variant,” see Analog.

“Vector” refers to a small carrier molecule into which a DNA sequence can be inserted for introduction into a new host cell where it will be replicated and, in some cases, expressed (in which case it can be termed an “expression vector”). Vectors are examples of gene delivery vehicles. Exemplary vectors include viruses, plasmids, cosmids, yeast artificial chromosomes, and human artificial chromosomes.

“Western blotting” or “Western analysis” refers to a method for detecting specific protein sequences. For example, the process can be performed by electrophoresing a protein mixture in a denaturing agarose or acrylamide gel, transferring the mixture onto a membrane, and incubating it with an antibody raised against the protein of interest.

Other terms and phrases are defined in other portions of this application.

C. General Discussion of Nucleic Acids and Polypeptides Related to GPR 38

The present invention includes nucleic acid and amino acid polymers, such as DNA and proteins, directed to GPR 38. Such polymers can be cloned, expressed, isolated, purified, and otherwise obtained or manipulated according to routine methods known in the art in view of the present application.

Expression Profile of 38

Immunohistochemistry analysis (coupled with H&E counterstain) as described herein has separated expression levels from 0 to 4, where 0=negative, 1=blush, 2=faint, 3=moderate, and 4=strong. Based on such analyses, GPR 38 immunohistochemical staining was strong in one or more cell types in the following normal human peripheral tissues: adrenal, bladder, pancreas, skin, small intestine, spleen, thymus, thyroid, tonsil, and uterus. GPR 38 staining was strong in the following normal human brain tissues: caudate, cerebellum, cortex, hypothalamus, medulla, pituitary, thalamus, and hippocampus. Moderate staining for GPR 38 was observed in the following normal human peripheral tissues: breast, colon, heart, kidney, liver, lung, lymph node, ovary, prostate, skeletal muscle, and stomach. GPR 38 staining was also moderate in the following normal human brain tissues: amygdala, basal nucleus of Meynert, putamen, and substantia nigra. Faint staining for GPR 38 was identified in normal human testis.

Again using immunohistochemistry for GPR 38 (coupled with H&E counterstain), the following differences in staining between diseased human samples and normals were identified; comparison of staining in diseased tissues versus normal tissues was made during analysis of diseased tissues, and identified differences in staining (increased or decreased relative to staining in normal tissues) indicate that a change from one scale value to another was found:

Brain, Alzheimer's disease (cerebral cortex): In samples of Alzheimer's disease, neurons were generally negative for staining, with very rare neurons showing faint positivity. Astrocytes were negative or showed faint positivity. Compared to normal cortex, which showed moderate staining of neurons and moderate to strong staining of astrocytes, samples of Alzheimer's disease showed reduced staining of neurons and astrocytes, and focal, faint to strong staining within plaques (structure not present in normal brain).

Brain, Parkinson's disease (substantia nigra): In samples of Parkinson's disease, pigmented neurons were negative for staining. Nonpigmented neurons were predominantly negative, showing rare, faint positivity. Neuropil was negative or showed faint positivity. The level of staining in pigmented and nonpigmented neurons and in neuropil was less than that in the normal substantia nigra, in which staining in pigmented neurons was moderate, with occasional staining extending down neuronal processes.

Brain, infarct (cerebral cortex and caudate): In samples of brain infarct, injured neurons were negative, and astrocytes were negative. Compared to normal samples, which showed moderate staining of neurons and moderate to strong staining of astrocytes, samples of brain infarct showed reduced staining of neurons and astrocytes, and faint staining of reactive endothelial cells (cell alteration not present in normal tissue).

Colon, ulcerative colitis: In samples of ulcerative colitis, surface epithelium was essentially negative for staining, with focal faint staining identified occasionally. Neuroendocrine (APUD) cells were negative, and ganglion cells in enteric plexuses were negative to faint. Compared to normal colon, which showed moderate staining of the surface epithelium and neuroendocrine cells, and faint staining of enteric ganglion cells, samples of ulcerative colitis showed loss of staining of the epithelium, neuroendocrine cells, and of ganglion cells, and showed staining of subsets of reactive fibroblasts and transformed lymphoid cells (cell alterations not present in normal tissue).

Small intestine, Crohn's disease: In samples of Crohn's disease, absorptive epithelium (enterocytes) was negative for staining. Neuroendocrine cells were negative or showed faint to moderate positivity. Compared to normal samples, which showed negative to faint staining of enterocytes and strong staining of neuroendocrine (APUD) cells, samples of small intestine from patients with Crohn's disease showed reduced staining of epithelium and neuroendocrine cells, as well as faint staining of eosinophils (eosinophils are not generally present in normal tissue).

Lymph node, Hodgkin's disease: In samples of lymph node from patients with Hodgkin's disease, Reed-Sternberg cells and Reed-Sternberg variants (not present in normal lymph node) showed focal blush to moderate staining. Reactive lymphoid cells (cell alteration not present in normal lymph node) showed focal punctate staining.

Brain, glioblastoma: In samples of glioblastoma, neoplastic cells (altered cells not present in normal brain) showed focal, strong staining.

Carcinomas, including breast, colon, lung (small cell and adenocarcinoma), ovarian, pancreatic (small cell and adenocarcinoma), and prostate showed variable, faint to strong GPR 38 immunohistochemical staining of carcinoma cells (altered cells not present in normal tissue).

1. Discussion Directed Generally to Nucleic Acids

Nucleic Acids Generally

The present invention comprises polynucleotides that encode a GPR 38 polypeptide, or an analog, portion, derivative, mimetic, or variant thereof. Such polynucleotides can be single-stranded (coding or antisense) or double-stranded, and may be DNA (e.g., genomic, cDNA or synthetic) or RNA molecules. Additional coding or non-coding sequences may, but need not, be present within the polynucleotides of the present invention, and the GPR 38 polynucleotides can, but need not, be linked to other molecules or support materials.

Analogs/Variants

The polynucleotides specifically recited herein, as well as full-length polynucleotides comprising such sequences, other portions of full-length polynucleotides, and sequences complementary to at least a portion of such full-length molecules, are specifically encompassed by the present invention. In addition, GPR 38 homologs from other species are specifically contemplated, and may generally be prepared as described herein for the other sequences identified herein, or as would be apparent to those skilled in the art in view of the present application. Analogs and variants of GPR 38 have been reported. Splice variants MTLR1A and MTLR1B were reported by Feighner, S. D., et al., supra, Science 284(5423):2184-2188 (1999) (PMID: 10381885), while dog and rabbit motilin receptor orthologs were reported in WO0132710.

Certain variants encode a polypeptide that retains the motilin binding activity or secondary messenger signaling of GPR 38 at a level that is not substantially lower than the level stimulated by the native protein. The effect on the properties of the encoded polypeptide may generally be assessed as described herein, or as would be apparent to those skilled in the art in view of the present application. Preferred variants contain nucleotide substitutions, deletions, insertions, and modifications at no more than about 20%, preferably at no more than about 10%, of the nucleotide positions. Certain variants are substantially homologous to a native gene, or a portion or complement thereof. Such polynucleotide variants are capable of hybridizing under moderately stringent conditions or other appropriate stringency conditions, as desired, to a naturally occurring DNA sequence encoding a GPR 38 protein (or a complementary sequence). Such hybridizing DNA sequences are also within the scope of this invention.

As a result of the degeneracy of the genetic code there are many nucleotide sequences that encode a polypeptide as described herein. Some of these polynucleotides bear low homology to the nucleotide sequence of any native gene. Nonetheless, polynucleotides that vary due to differences in codon usage are specifically contemplated by the present invention.

Related Genes

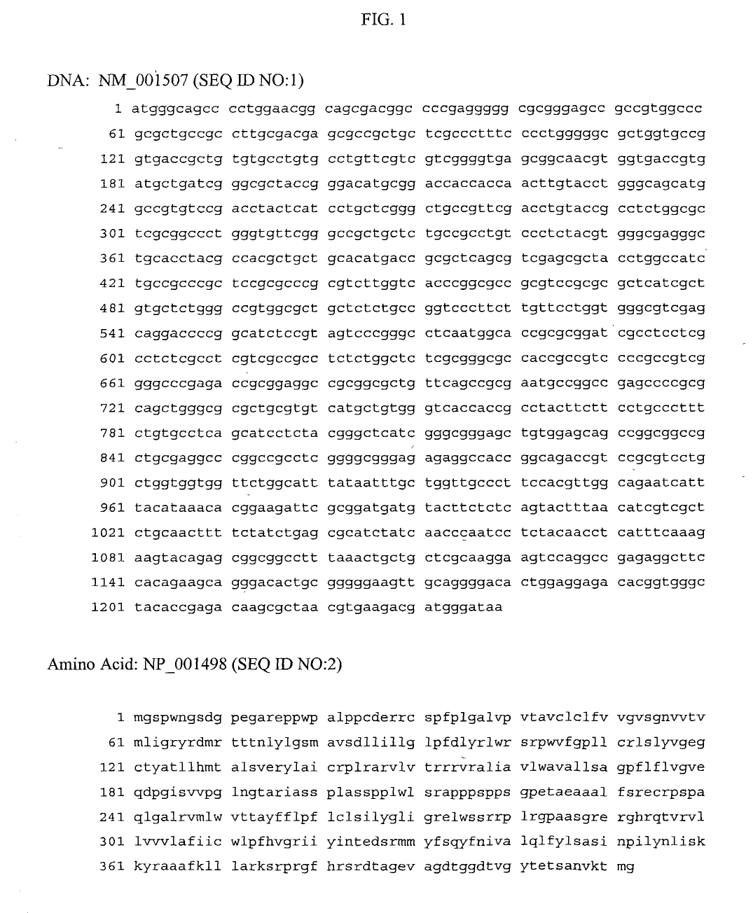

The present invention also provides compositions and methods for identifying and cloning other genes related to GPR 38. Generally, such genes can be recombinant or non-recombinant and comprise a sequence having at least about 70% identity over a stretch of at least about 30 nucleotides to the nucleic acid sequence of SEQ ID NO: 1, FIG. 1. Such related genes can be identified and obtained, for example, either through traditional hybridization and cloning techniques using the polynucleotide of SEQ ID NO: 1, FIG. 1, or other polynucleotide encoding GPR 38 as a probe, or by searching databases such as the GenBank family of databases or the LifeSpan subscription GPCR or LifeSource™ databases.

Nucleic Acids Defined by Hybridization

The present invention further relates to polynucleotides that hybridize to the described sequences herein, for example the sequence of SEQ ID NO: 1, FIG. 1, typically where there is at least about 70%, preferably at least about 90%, and more preferably at least about 95% identity between the sequences. (For example, about 70% identity would include within such definition a 70 bp fragment taken from a 100 bp polynucleotide, or a target sequence that contains non-complementary sequences in addition to the region having the about 70% identity.) The present invention particularly relates to polynucleotides that hybridize under stringent conditions to the herein-described polynucleotides. Such polynucleotides typically comprise at least about 95% and preferably at least about 97% identity, up to complete identity, between the sequences. The polynucleotides that hybridize to the hereinabove described polynucleotides in a preferred embodiment encode enzymes that retain substantially the same ability to bind motilin or substantially the same secondary messenger actions of GPR 38.

Alternatively, the polynucleotide may have at least 15 bases, preferably at least 30 bases, and more preferably at least 50 bases that hybridize to any part of a polynucleotide of the present invention and which has an identity thereto, and which may or may not retain biological activity. For example, such polynucleotides may be employed as probes for the polynucleotides of SEQ ID NO: 1, FIG. 1, such as for recovery of the polynucleotide or as a diagnostic probe or as a PCR primer.

Thus, the present invention comprises polynucleotides having at least about 70% identity, preferably at least about 90% identity and more preferably at least about 95% identity to a polynucleotide that encodes GPR 38, for example either the polynucleotide of SEQ ID NO: 1, or the polypeptide of SEQ ID NO: 2, FIG. 1, as well as fragments thereof, which fragments have at least 15 bases, preferably at least 30 bases, more preferably at least about 50 bases, and most preferably fragments having up to at least about 150 bases or greater, which fragments are at least about 90% identical, preferably at least about 95% identical, and most preferably at least about 97% identical to any portion of a polynucleotide of the present invention.

Probes

GPR 38 DNA and other nucleic acid sequences of the present invention, including analogs and the like of SEQ ID NO: 1, FIG. 1, may, in view of the present application, be isolated using any of a variety of hybridization or amplification techniques that are well known to those of ordinary skill in the art. For example, probes or primers may be designed based on the GPR 38 sequences provided herein or elsewhere, and may be purchased or synthesized. Libraries from any suitable tissue (e.g., breast, ovary, prostate, skeletal muscle, stomach, testis, amygdala, basal nucleus of Meynert, cortex, and substantia nigra), particularly those involved in Alzheimer's disease or Parkinson's disease, may be screened. An amplified portion or partial cDNA molecule may then be used to isolate a full-length gene from a genomic DNA library or from a cDNA library, using well known techniques in view of the present application. As another example, a full-length gene can be constructed from multiple PCR fragments. A nucleic acid sequence corresponding to the native GPR 38 polypeptide is provided in SEQ ID NO: 1, FIG. 1.

GPCR Polynucleotides in Vectors

The present invention also includes polynucleotides wherein the coding sequence for the desired polypeptide is fused in the same reading frame to a polynucleotide sequence that aids in expression and secretion of a polypeptide from a host cell, for example, a leader sequence that functions as a secretory sequence for controlling transport of a polypeptide from the cell. The sequences can be a part of various vectors, which are also discussed further elsewhere herein, or would be apparent to those skilled in the art in view of the present application. The polypeptide having a leader sequence is a preprotein and may have the leader sequence cleaved by the host cell to form the mature form of the polypeptide. The polynucleotides may also code for a proprotein which is the mature protein plus additional 3′ or 5′ amino acid residues. A mature protein having a prosequence is a proprotein and is an inactive form of the protein. Once the prosequence is cleaved an active mature protein remains. Thus, for example, the polynucleotide of the present invention may encode a mature protein, or a protein having a prosequence or for a protein having both a prosequence and a presequence (e.g., leader sequence).

The polynucleotides of the present invention may also have the coding sequence fused in frame to a marker sequence that assists purification of the polypeptide of the present invention. The marker sequence may be, for example, a hexa-histidine tag supplied by a pQE-9 vector to provide for purification of the mature polypeptide fused to the marker in the case of a bacterial host, or a hemagglutinin (HA) tag when a mammalian host, e.g., COS-7 cells, is used. The HA tag corresponds to an epitope derived from the influenza hemagglutinin protein, Wilson, I., et al., Cell, 37:767 (1984).

Expression Profile Based on mRNA

As noted above, the nucleic acids of the invention include mRNA encoding GPR 38 such as mRNA corresponding to SEQ ID NO: 1, FIG. 1, or encoding the polypeptide of SEQ ID NO: 2, FIG. 1. Accordingly, the present invention also provides compositions and methods for localizing mRNA coding for the polypeptide of the invention. Such mRNAs can be localized, if desired, as follows.

Human multiple tissue and cancer cell line blots containing approximately 2 μg of poly(A)⁺ RNA per lane, Clontech (Palo Alto, Calif.) can be radiolabeled with [α³² P] dATP, e.g., using the Amersham Rediprime random primer labeling kit (RPN1633, Piscataway, N.J.). Prehybridization and hybridization can be performed at 65° C. in 0.5 M Na₂HPO₄, 7% SDS, 0.5M EDTA (pH 8.0). Washes can be conducted, e.g., at 65° C. with two initial washes in 2×SSC, 0.1% SDS for 40 min followed by a subsequent wash in 0.1×SSC, 0.1% SDS for 20 min. Membranes are then exposed at −70° C. to X-Ray film (Kodak) in the presence of intensifying screens. If desired, studies using cDNA libraries and Southerns can be performed with selected clones of nucleic acids having the nucleotide sequence of GPR 38 such as the sequence set forth in SEQ ID NO: 1, FIG. 1, or other polynucleotide sequences disclosed herein to examine their expression in certain cell subsets.

Two prediction algorithms that take advantage of the patterns of conservation and variation in multiply-aligned sequences, Rost and Sander, Proteins 19:55-72 (1994), and DSC, King and Sternberg, Protein Sci. 5:2298-2310 (1996) can be used if desired for this and other aspects of the invention where appropriate; other algorithms are also suitable. Alternatively, two appropriate primers are selected and RT-PCR is used on an appropriate mRNA sample selected for the presence of message to produce a cDNA, e.g., a sample that expresses the gene. Full-length clones can be isolated by hybridization of cDNA libraries from appropriate tissues pre-selected by PCR signal. mRNA can be assayed by appropriate technology, e.g., PCR, immunoassay, hybridization, or otherwise. Tissue and organ cDNA preparations are available, e.g., from Clontech, Palo Alto, Calif.

Samples for human mRNA isolation and determination of distribution of expression may include any desired tissue, such as those discussed elsewhere herein. Suitable analytic approaches include Northern analysis, in situ hybridization, solution hybridization, and high density array.

2. Discussion Directed Generally to Polypeptides

Polypeptides Generally

The present invention further relates to polypeptides having an amino acid sequence of GPR 38, such as the sequence set forth in SEQ ID NO: 2, FIG. 1, including analogs, mimetics, fragments, derivatives, and the like of such polypeptides. The polypeptides may be recombinant, natural or synthetic. The polypeptides include (i) polypeptides in which one or more of the amino acid residues are substituted with a conserved or non-conserved amino acid residue (preferably a conserved amino acid residue) and such substituted amino acid residue may or may not be one encoded by the genetic code, (ii) polypeptides in which one or more of the amino acid residues includes a substituent group, (iii) polypeptides in which the mature polypeptide is complexed (e.g., fused or otherwise bonded) with another compound, such as a compound to increase the half-life of the polypeptide (for example, polyethylene glycol), (iv) polypeptides in which additional amino acids are fused to the mature polypeptide, and (v) polypeptides in which a fragment of the polypeptide is soluble, e.g., not membrane bound, yet still binds its specific ligand. Preparing and using such analogs, etc., are within the scope of those skilled in the art in view of the present application.

The polypeptides additionally include polypeptides that have at least about 70% identity, more preferably at least about 90% identity to the polypeptide of GPR 38, and still more preferably at least about 95% identity to the polypeptide of GPR 38. The polypeptides also include portions of such polypeptides with such portion of the polypeptide generally containing at least 30 amino acids and more preferably at least 50 amino acids. As known in the art “similarity” between two polypeptides is determined by comparing the amino acid sequence and its conserved amino acid substitutes of one polypeptide to the sequence of a second polypeptide.

Portions of the polypeptides of the present invention can be used to produce corresponding full-length polypeptides by peptide synthesis; therefore, the fragments may be employed as intermediates for producing the full-length polypeptides. Similarly, portions of the polynucleotides of the present invention may be used to synthesize full-length polynucleotides of the present invention.

Expression Profiles Based on Proteins

An expression profile of GPR 38 can be made using traditional approaches such as Western blotting, immunohistochemistry analysis, protein array, ligand-binding studies, radioimmunoassay (RIA), and high performance liquid chromatography (HPLC). Such profiles can be made as described in the Examples or otherwise, for example as set forth in the following paragraphs.

Screening for GPCR Activity

The activity or functionality of GPR 38 may be measured using any of a variety of functional assays in which activation of the receptor in question results in an observable change in the level of some second messenger system, including but not limited to adenylyl cyclase, calcium mobilization, arachidonic acid release, ion channel activity, inositol phospholipid hydrolysis, or guanylyl cyclase. Heterologous expression systems utilizing appropriate host cells to express the nucleic acid of the subject invention are used to obtain the desired second messenger coupling. Receptor activity may also be assayed in an oocyte expression system.

Protein Purification

The polypeptides can be purified by standard methods, including but not limited to salt or alcohol precipitation, preparative disc-gel electrophoresis, isoelectric focusing, high pressure liquid chromatography (HPLC), reversed-phase HPLC, gel filtration, cation and anion exchange, partition chromatography, and countercurrent distribution. Suitable purification methods will be readily apparent to those skilled in the art in view of the present application and are disclosed, e.g., in Guide to Protein Purification, Methods in Enzymology, Vol. 182, M. Deutscher, Ed., Academic Press, New York, N.Y. (1990). Purification steps can be followed as part of carrying out assays for ligand binding activity. Particularly where GPR 38 is being isolated from a cellular or tissue source, it is preferable to include one or more inhibitors of proteolytic enzymes in the assay system, such as phenylmethylsulfonyl fluoride (PMSF).

D. Certain Assays, Antibodies, Probes, Therapeutics, and Other Systems and Aspects, of the Invention

1. Identifying Binding Agents and Modulating Agents

Methods for Identifying Binding Agents and Modulating Agents

The present invention further provides systems and methods for identifying substances or compounds that bind to or modulate the expression or activity of GPR 38 polypeptide. Suitable assays include ligand binding (membrane binding or slice binding); expression based systems such as CART-technology (Constitutively Activated Receptor Technology, Arena Pharmaceuticals, San Diego, Calif.) for screening chemical libraries of small molecule compounds to identify novel drugs; expression based systems such as AequoScreen (EuroScreen), an aequorin-based assay for high throughput screening of chemical libraries of small molecule compounds to identify novel drugs; expressing GPR 38 in recombinant cell lines for drug screening (Euroscreen, Brussels, Belgium) or functional analysis; Northern, Western and Southern blots; in situ hybridization and solution hybridization; protein arrays, nucleotide arrays, spectral analysis, radioimmunoassay, immunoassay, immunodetection; therapeutic antibodies targeting GPR 38; computer modeling; and, photoaffinity labeling to determine the binding pocket of GPR 38. Several of these assays, as well as other assays, are discussed elsewhere herein.

Biological Activity Assay

To evaluate the effect of a candidate modulating agent on GPR 38 polypeptide activity or functionality, a biological activity assay may be performed wherein the candidate modulating agent is added to the incubation mixture. Briefly, the reaction components, which include the composition to be tested and GPR 38 polypeptide or a polynucleotide encoding GPR 38 polypeptide, are incubated under conditions sufficient to allow the components to interact. Subsequently, the effect of the composition or component on GPCR biological activity or on the level of polynucleotide encoding GPR 38 is measured. The observed effect on GPR 38 may be either inhibitory or stimulatory. The increase or decrease in GPCR biological activity can be measured by, for example, adding a radioactive compound such as ³²P-ATP to the mixture of components, and observing radioactive incorporation into a suitable substrate for GPR 38, to determine whether the compound inhibits or stimulates GPCR biological activity. A polynucleotide encoding GPR 38 may be inserted into an expression vector and the effect of a composition on transcription of GPR 38 mRNA can be measured, for example, by Northern blot analysis.

Within such assays, the candidate agent may be preincubated with GPR 38 polypeptide before addition of ATP and substrate or the substrate may be preincubated with the candidate agent before the addition of GPR 38. Further variations include adding the candidate agent to a mixture of GPR 38 polypeptide and ATP before the addition of substrate, or to a mixture of substrate and ATP before the addition of GPR 38 polypeptide. Any of these assays can further be modified by removing the candidate agent after the initial preincubation step. In general, a suitable amount of antibody or other candidate agent for use in such an assay ranges from about 0.1 μM to about 10 μM. The effect of the agent on GPR 38 biological activity may then be evaluated by quantifying the change in the amount or activity of the substrate, and comparing the level of biological activity with that achieved using the GPR 38 polypeptide without the addition of the candidate agent.

GPR 38 biological activity may also be measured, for example, in whole cells transfected with a reporter gene whose expression is dependent upon the biological activity of GPR 38 or the biological activity of a substrate of GPR 38. For example, polynucleotides encoding GPR 38 polypeptide and a substrate may be cotransfected into a cell. Following activation or modulation of GPR 38 activity, the substrate may then be immunoprecipitated, and its activity evaluated in an in vitro assay. Alternatively, cells may be transfected with an ATF2-dependent promoter linked to a reporter gene such as luciferase. In such a system, expression of the luciferase gene depends upon activation of ATF2 by p38, which may be achieved by the biological activity of GPR 38 polypeptide or the biological activity of a substrate of GPR 38. Candidate modulating agents may be added to the system to evaluate their effect on GPR 38 polypeptide activity.

Alternatively, a whole-cell (hybrid) system may employ only the transactivation domain of ATF2 fused to a suitable DNA-binding domain, such as GHF-1 or GAL4. The reporter system may then comprise the GH-luciferase or GAL4-luciferase plasmid. Candidate GPR 38 protein modulating agents may then be added to the system to evaluate their effect on ATF2-specific gene activation.

Biological functionality can also be assayed using methods similar to those described herein as well as using methods known in the art in view of the present application.

Supplying Biological Activity of Functionality of the GPCR

The present invention also provides compositions, methods, and the like for using GPR 38 to bind motilin or otherwise supply biological activity or functionality of GPR 38. In general, the amounts of the reaction components may range from about 0.1 μg to about 10 μg of GPR 38 polypeptide, from about 0.1 μg to about 10 μg of motilin or other desired substrate to generally provide an excess of ligand over receptor in any given reaction (in the absence of mechanisms of receptor down regulation).

Using Biological Activity or Functionality of the GPCR

The present invention additionally provides compositions, methods, and the like for using the binding of motilin or other suitable substrate by GPR 38 to treat, inhibit or diagnose certain diseases such as those recited elsewhere herein.

2. Systems and Methods for Screening Systems and Methods For Screening for Agonists and Antagonists

a. Generally

Screening for agonists and Antagonists—Purposes of Same

The invention provides for the discovery of selective agonists and antagonists of GPR 38 described herein that can be useful in treatment and management of a variety of diseases including Alzheimer's disease and Parkinson's disease, inflammatory bowel diseases including ulcerative colitis and Crohn's disease, Hodgkin's disease, glioblastoma and breast carcinoma, colon carcinoma, lung carcinoma (small cell and adenocarcinoma), pancreatic carcinoma (small cell and adenocarcinoma), ovarian carcinoma, and prostate carcinoma. Several agonist and antagonists have been reported for GPR 38. Reported agonists include motilin itself, as well as erythromycin, ABT-229, EM574 (an erythromycin derivative), EM-573 (an erythromycin derivative), GM-611, and SK-896. Reported antagonists include RWJ 68023 (a non-peptide antagonist, GM-109 (a peptide antagonist), MOT 1-12[CH(2)NH](10-12), and certain cyclopentene derivatives, see U.S. Pat. No. 5,972,939.

Thus, the receptor biological activity or functionality of GPR 38 can be employed in screening systems to identify agonists or antagonists of the receptor. Essentially, these systems provide methods for bringing together the GPCR, an appropriate known ligand, including ligand for which the GPCR is specific, such as motilin for GPR 38, and a sample to be tested for the presence of an agonist or antagonist.

Constitutively Active Receptor for Screening for Antagonists

The use of a constitutively active receptor encoded by GPR 38 either occurring naturally without further modification or after appropriate point mutations, deletions or the like, allows screening for antagonists and in vivo use of such antagonists to attribute a role to GPR 38 without prior knowledge of the endogenous ligand.

Screening for Receptor Diversity

Use of the nucleic acids further provides for elucidation of possible receptor diversity and of the existence of multiple subtypes within a family of receptors of which GPR 38 is a member.

At least two typical types of screening systems can be used, a labeled-ligand binding assay and a functional assay.

b. Labeled Ligand Assays

Labeled Ligand Assays

A labeled ligand for use in the binding assay can be obtained by labeling motilin or other chosen ligand or a known agonist or antagonist of the specific ligand with a measurable moiety as described herein, or as would be apparent to those skilled in the art in view of the present application. Various labeled forms of desired ligand may be available commercially or can be generated using standard techniques in view of the present application.

Typically, a given amount of GPR 38 is contacted with increasing amounts of a labeled ligand, such as motilin, and the amount of the bound labeled ligand is measured after removing unbound labeled ligand by washing. As the amount of the labeled ligand is increased, a point is eventually reached at which all receptor binding sites are occupied or saturated. Specific receptor binding of the labeled ligand is abolished by a large excess of unlabeled ligand.

An assay system can be used in which non-specific binding of the labeled ligand to the sample is minimal. Non-specific binding is typically less than about 50%, preferably less than about 15%, and more preferably less than about 10% of the total binding of the labeled ligand. In some cases, the non-specific binding of a ligand to a sample may be greater than about 50% of total binding if the level of receptor expression by the sample is very low.

In principle, a binding assay of the invention can be carried out using a soluble receptor of the invention, e.g., following production and refolding by standard methods from an E. coli expression system, and the resulting receptor-labeled ligand complex could be precipitated, e.g., using an antibody against the receptor. The precipitate can then be washed and the amount of the bound labeled ligand measured.

Alternatively, a nucleic acid encoding GPR 38 can be transfected into an appropriate host cell, whereby the receptor will become incorporated into the membrane of the cell. A membrane fraction can then be isolated from the cell and used as a source of the receptor for assay. Preferably, specific binding of the labeled ligand to a membrane fraction from the untransfected host cell will be negligible.

he binding assays of this invention can be used to identify both specific ligand agonists and specific ligand antagonists because both will interfere with the binding of the labeled ligand to the receptor.

Labeled Ligand Assay—Basic Binding Assay

In a basic binding assay, a suitable method for identifying a specific ligand agonist or specific ligand antagonist can comprise:

(a) contacting a GPCR having an amino acid sequence of GPR 38 such as that defined by SEQ ID NO: 2, FIG. 1 or an analog, etc., thereof, in the presence of a known amount of labeled specific ligand with a sample to be tested for the presence of an agonist or antagonist; and

(b) measuring the amount of labeled specific ligand bound to the receptor; whereby a specific ligand agonist or antagonist in the sample is identified by measuring substantially reduced binding of the labeled specific ligand to GPR 38, compared to what would be measured in the absence of such agonist or antagonist.

The methods can further comprise:

(c) Contacting GPR 38 in the presence of a known amount of labeled specific ligand with a compound identified as an agonist or antagonist for the specific ligand in steps (a) and (b); and

(d) Measuring the amount of labeled specific ligand bound to the receptor; whereby the agonist or antagonist specific for GPR 38 or specific ligand is identified by measuring substantially undiminished binding of the labeled specific ligand to the receptor, compared to what would be measured in the absence of such agonist or antagonist. Determining whether a particular molecule inhibiting the binding of the labeled specific ligand to GPR 38 is an antagonist or an agonist can then be determined in a second assay such as a functional assay. The functionality of such agonists and antagonists identified in the binding assay can be determined, for example, in cellular and animal models.

c. Functional Assays for Antagonists or Agonists of GPR 38

Functional Assays

In cellular models, parameters for intracellular activities mediated by GPCRs can be monitored for antagonistic or agonistic activities. Such parameters include but are not limited to intracellular second messenger pathways activated via the GPCRs, changes in cell growth rate, secretion of hormones, etc., using published methods. Examples of such methods include measurement of the effects of a putative ligand on receptor-mediated binding of motilin, or on the effects of the putative ligand on observable biological activities of motilin.

Agonists and antagonists of GPCRs may also be identified directly by using functional assays. An agonist or antagonist may or may not directly inhibit or enhance specific ligand binding to GPCRs.

Functional Assays —Measuring Antagonist Activity

In addition to the methods described above, activities of an agonist or antagonist may be measured in cellular models for altered motilin binding by GPR 38, or on the effects of the putative ligand on observable biological activities of motilin, for example biological activities of motilin related to Alzheimer's disease and Parkinson's disease, inflammatory bowel diseases including ulcerative colitis and Crohn's disease, Hodgkin's disease, glioblastoma, and breast carcinoma, colon carcinoma, lung carcinoma (small cell and adenocarcinoma), pancreatic carcinoma (small cell and adenocarcinoma), ovarian carcinoma, and prostate carcinoma.

3. Systems and Methods for Screening for GPR 38 Polypeptide of Polynucleotide

Screening for Polypeptide of Polynucleotide

As noted elsewhere herein, the present invention provides GPR 38 polypeptide and analogs, etc., thereof The invention also provides systems and methods for detecting such polypeptides in a sample. The assays are typically based on the detection of antigens or epitopes displayed by GPR 38 or antibodies produced against GPR 38, but also include nucleic acid based assays (typically based upon hybridization).

a. Assays Based On GPR 38 Polypeptides

Screening for/with Polypeptide

Many assays are characterized by the ability of GPR 38 polypeptides to be bound by antibodies generated against them and the ability of antibodies produced against such proteins to bind to antigens or epitopes of GPR 38 in a sample. Some exemplary assays are described below and elsewhere herein.

List of Assays

A variety of assays can detect antibodies that bind specifically to the desired protein from a sample, or to detect the desired protein bound to one or more antibodies from the sample. Exemplary assays are described in detail in Antibodies: A Laboratory Manual, Harlow and Lane (eds.), Cold Spring Harbor Laboratory Press (1988). Representative examples of such assays include: countercurrent immuno-electrophoresis (CIEP), radioimmunoassays, radioimmunoprecipitations, enzyme-linked immunosorbent assays (ELISA), dot blot assays, inhibition or competition assays, sandwich assays, immunostick (dip-stick) assays, simultaneous assays, immunochromatographic assays, immuunofiltration assays, latex bead agglutination assays, immunofluorescent assays, biosensor assays, and low-light detection assays, see U.S. Pat. Nos. 4,376,110 and 4,486,530; WO 94/25597; WO/25598; see also Antibodies: A Laboratory Manual, supra.

Enzyme-Linked Immunosorbent Assays (ELISA)

One assay for the detection of GPR 38 is a sandwich assay such as an enzyme-linked immunosorbent assay (ELISA). In one preferred embodiment, the ELISA comprises the following steps: (1) coating GPR 38 polypeptide onto a solid phase, (2) incubating a sample suspected of containing anti-GPR 38 antibodies with the polypeptide coated onto the solid phase under conditions that allow the formation of an antigen-antibody complex, (3) adding an anti-antibody (such as anti-IgG) conjugated with a label to be captured by the resulting antigen-antibody complex bound to the solid phase, and (4) measuring the captured label and determining therefrom whether the sample contains anti-GPR 38 antibodies.

Immunofluorescence Assay

A fluorescent antibody test (FA-test) uses a fluorescently labeled antibody able to bind to one of the proteins of the invention. For detection, visual determinations are made by a technician using fluorescence microscopy, yielding a qualitative result. In one embodiment, this assay is used for the examination of tissue samples or histological sections.

Bead Agglutination Assays

n latex bead agglutination assays, antibodies to one or more of the proteins of the present invention are conjugated to latex beads. The antibodies conjugated to the latex beads are then contacted with a sample under conditions permitting the antibodies to bind to desired proteins in the sample, if any. The results are then read visually, yielding a qualitative result. In some embodiments, as with certain other assays, this format can be used in the field for on-site testing.

Enzyme Immunoassays

Enzyme immunoassays (EIA) include a number of different assays that can use the antibodies described in the present application. For example, a heterogeneous indirect EIA uses a solid phase coupled with an antibody of the invention and an affinity purified, anti-IgG immunoglobulin preparation. The solid phase can be a polystyrene microtiter plate. The antibodies and immunoglobulin preparation are then contacted with the sample under conditions permitting antibody binding, which conditions are well known in the art. The results of such an assay can be read visually, but are preferably read using a spectrophotometer, such as an ELISA plate reader, to yield a quantitative result. An alternative solid phase EIA format includes plastic-coated ferrous metal beads able to be moved during the procedures of the assay by means of a magnet. Yet another alternative is a low-light detection immunoassay format. In this highly sensitive format, the light emission produced by appropriately labeled bound antibodies are quantified automatically. Preferably, the reaction is performed using microtiter plates.

In an alternative embodiment, a radioactive tracer is substituted for the enzyme-mediated detection in an EIA to produce a radioimmunoassay (RIA).

Sandwich Assay

In a capture-antibody sandwich enzyme assay, the desired protein is bound between an antibody attached to a solid phase, preferably a polystyrene microtiter plate, and a labeled antibody. Preferably, the results are measured using a spectrophotometer, such as an ELISA plate reader. This assay is one preferred embodiment for the present invention.

Sequential and Simultaneous Assays

In a sequential assay format, reagents are allowed to incubate with the capture antibody in a stepwise fashion. The test sample is first incubated with the capture antibody. Following a wash step, incubation with the labeled antibody occurs. In a simultaneous assay, the two incubation periods described in the sequential assay are combined. This eliminates one incubation period plus a wash step.

Immunostick (Dip-Stick) Assays

A dipstick/immunostick format is essentially an immunoassay, with the exception that the solid phase is a polystyrene paddle or dipstick instead of a polystyrene microtiter plate. Reagents are the same and the format can either be simultaneous or sequential.

Immunochromatographic Assays

In a chromatographic strip test format, a capture antibody and a labeled antibody are dried onto a chromatographic strip, which is typically comprising nitrocellulose or nylon of high porosity bonded to cellulose acetate. The capture antibody is usually spray dried as a line at one end of the strip. At this end, there is an absorbent material that is in contact with the strip. At the other end of the strip, the labeled antibody is deposited in a manner that prevents it from being absorbed onto the membrane. Usually, the label attached to the antibody is a latex bead or colloidal gold. The assay may be initiated by applying the sample immediately in front of the labeled antibody.

Immunofiltration Assays

Immunofiltration/immunoconcentration formats combine a large solid-phase surface with directional flow of sample/reagents, which concentrates and accelerates the binding of antigen to antibody. In a preferred format, the test sample is preincubated with a labeled antibody, and then applied to a solid phase such as fiber filters, nitrocellulose membranes, or the like. The solid phase can also be precoated with latex or glass beads coated with capture antibody. Detection of analyte is the same as that in a standard immunoassay. The flow of sample/reagents can be modulated by either vacuum or the wicking action of an underlying absorbent material.

Biosensor Assays

A threshold biosensor assay is a sensitive, instrumented assay amenable to screening large numbers of samples at low cost. In one embodiment, such an assay comprises the use of light-addressable potentiometric sensors wherein the reaction involves the detection of a pH change due to binding of the desired protein by capture antibodies, bridging antibodies, and urease-conjugated antibodies. Upon binding, a pH change is effected that is measurable by translation into electrical potential (μvolts). The assay typically occurs in a very small reaction volume, and is very sensitive; the reported detection limit of the assay is 1,000 molecules of urease per minute.

b. Assays Based On GPR 38 Polynucleotides

Screening for/with Polynucleotides—Probes

Polynucleotides, including fragments thereof, as described herein can be used as hybridization probes for a cDNA or a genomic library to isolate full-length DNA and to isolate other DNAs that have a high sequence similarity to GPR 38 or similar biological activity to GPR 38. Probes of this type preferably have at least 10, preferably at least 15, and even more preferably at least 30 bases, and may contain, for example, at least 50 or more or 150 or more bases. The probe may also be used to identify a DNA clone corresponding to a transcript, including a full-length transcript, and a genomic clone or clones that contain the gene including regulatory and promoter regions, exons, and introns. An example of an assay comprising a screen comprises isolating the coding region of the gene by using a DNA sequence of GPR 38 such as a suitable portion of the sequence set forth in SEQ ID NO: 1, FIG. 1, to synthesize an oligonucleotide probe. Labeled oligonucleotides having a sequence complementary to or identical to that of the polynucleotides described herein can be used to screen a library of genomic DNA to determine to which members of the library the probe hybridizes.

Such probes can also be labeled with an analytically detectable reagent to facilitate identification of the probe. Useful reagents include, but are not limited, to radioactivity, fluorescent dyes, or enzymes capable of catalyzing the formation of a detectable product. The probes are thus useful to isolate complementary copies of DNA from other sources or to screen such sources for related sequences.

4. Antibodies

Antibodies Generated Against GPR 38

Antibodies against GPR 38 have been generated using peptides derived from the amino acid sequence of GPR 38 as antigens, then using traditional antibody generation techniques described below. The antibodies were then used to conduct immunohistochemistry and other analyses of a variety of tissue samples to determine GPR 38 expression in such tissues. The antigenic fragments were as follows: REPPWPALPPCDERRCS, SEQ ID NO: 3, SPPSGPETAEAAALFSREC, SEQ ID NO: 4, SSRRPLRGPAASGRERGHRQ, SEQ ID NO: 5, and RKSRPRGFHRSRDTAG, SEQ ID NO: 6. Commercially available antibodies can also be used for certain purposes related to GPR 38. Commercially available antibodies include GPR38 (D-19), sc-5450; GPR38-A (C-17), sc-5451; GPR38-B (H-15); and sc-5454, from Santa Cruz Biotechnology, Inc., Santa Cruz, Calif. The specification will now discuss a variety of antibody types, methods, uses, etc., related to GPR 38.

Antibodies Generally

In some embodiments, the present invention provides antibodies or similar binding partners directed to GPR 38, and ligands to GPR 38 or to the binding site of the antibodies. Compositions and uses for such antibodies and ligands are contemplated, including diagnostic, medicament, and therapeutic uses. Various diagnostic, medicament, and therapeutic uses for antibodies have been reviewed, for example, in Goldenberg et al., Semin. Cancer Biol., 1(3):217-225 (1990); Beck et al., Semin. Cancer Biol., 1(3):181-188 (1990); Niman, Immunol. Ser., 53:189-204 (1990); Endo, Nippon Igaku Hoshasen Gakkai Zasshi (Japan), 50(8):901-909 (1990); and, U.S. Pat. No. 6,214,984.

Recognized immunoglobulin genes include the kappa, lambda, alpha, gamma, delta, epsilon, and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Anti-Idiotypic Antibodies

The present invention also encompasses anti-idiotypic antibodies, polyclonal, monoclonal, and otherwise, that are produced using the antibodies described herein as antigens. These antibodies are useful because they may mimic the structures of the receptors.

Techniques for producing antibodies, including antibody fragments, include the following.

a. Antibody Preparation

(i) Polyclonal Antibodies

Antibody Prep—Polyclonal

Polyclonal antibodies are generally raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Antibody Prep—Adjuvants (All Abs)

Suitable adjuvants for the vaccination of animals for the production of polyclonal, monoclonal, and other antibodies include but are not limited to Adjuvant 65 (containing peanut oil, mannide monooleate, and aluminum monostearate); Freund's complete or incomplete adjuvant; mineral gels such as aluminum hydroxide, aluminum phosphate, and alum; surfactants such as hexadecylamine, octadecylamine, lysolecithin, dimethyldioctadecylammonium bromide, N,N-dioctadecyl-N′,N′-bis(2-hydroxymethyl) propanediamine, methoxyhexadecylglycerol, and pluronic polyols; polyanions such as pyran, dextran sulfate, poly IC, polyacrylic acid, and carbopol; peptides such as muramyl dipeptide, dimethylglycine, tuftsin, stress proteins, core-containing proteins from a positive stranded RNA virus, see U.S. Pat. No. 6,153,378; and, oil emulsions. The polypeptides could also be administered following incorporation into liposomes or other microcarriers. Information concerning adjuvants and various aspects of immunoassays are disclosed, e.g., in the series by P. Tijssen, Practice and Theory of Enzyme Immunoassays, 3rd Edition (1987), Elsevier, New York. Other useful references covering methods for preparing polyclonal antisera include Microbiology, Hoeber Medical Division, Harper and Row (1969); Landsteiner, Specificity of Serological Reactions, Dover Publications, New York (1962); and, Williams, et al., Methods in Immunology and Immunochemistry, Vol. 1, Academic Press, New York (1967).

Animals can be immunized against the antigen, immunogenic conjugates, or derivatives by combining 1 mg or 1 μg of the peptide or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein or through a different cross-linking reagent. Conjugates also can be made in recombinant cell culture as protein fusions. In addition, aggregating agents such as alum can be suitably used to enhance the immune response.

(ii) Monoclonal Antibodies

Antibody Prep—Monoclonal

Monoclonal antibodies are obtained from a population of substantially homogeneous antibodies, e.g., the individual antibodies comprising the population are identical except for possible naturally occurring mutations that may be present in minor amounts. For example, monoclonal antibodies can be made using the hybridoma method first described by Kohler and Milstein, Nature, 256:495 (1975), or can be made by recombinant DNA methods.

n the hybridoma method, a mouse or other appropriate host animal, such as a hamster, is immunized as hereinabove described to elicit lymphocytes that produce or are capable of producing antibodies that will bind specifically to the protein used for immunization. Alternatively, lymphocytes may be immunized in vitro. Lymphocytes then are fused with myeloma cells using a suitable fusing agent, such as polyethylene glycol, to form a hybridoma cell, Goding, Monoclonal Antibodies: Principles and Practice, pp. 59-103, Academic Press (1986).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium, for example murine myeloma lines, such as those derived from MOPC-21 and MPC-11 mouse tumors available from the Salk Institute Cell Distribution Center, San Diego, Calif. USA, and SP-2 cells available from the American Type Culture Collection, Rockville, Md. USA. Human myeloma and mouse-human heteromyeloma cell lines have also been described for the production of human monoclonal antibodies, Kozbor, J. Immunol., 133:3001 (1984); Brodeur et al., Monoclonal Antibody Production Techniques and Applications, pp. 51-63, Marcel Dekker, Inc., New York (1987).

Culture medium in which hybridoma cells are growing is assayed for production of monoclonal antibodies directed against the antigen. The binding specificity of monoclonal antibodies produced by hybridoma cells can be determined by immunoprecipitation or by an in vitro binding assay, such as radioimmunoassay (RIA) or enzyme-linked immunosorbent assay (ELISA). The binding affinity of the monoclonal antibody can, for example, be determined by the Scatchard analysis of Munson and Pollard, Anal. Biochem., 107:220 (1980).

After hybridoma cells are identified that produce antibodies of the desired specificity, affinity, or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-SEPHAROSE™, hydroxyapatite chromatography, gel electrophoresis, dialysis, or affinity chromatography.

DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which can then be transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Review articles on recombinant expression in bacteria of DNA encoding antibody include Skerra et al., Curr. Opinion in Immunol., 5:256-262 (1993), and Pluckthun, Immunol. Revs., 130:151-188 (1992).

MOABS—Combinatorial

In a further embodiment, antibodies or antibody fragments can be isolated from antibody phage libraries generated using the techniques described in McCafferty et al., Nature, 348:552-554 (1990), using the proper antigen such as CD11 a, CD18, IgE, or HER-2 to select for a suitable antibody or antibody fragment. Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling, Marks et al., Biotechnology, 10:779-783 (1992), as well as combinatorial infection and in vivo recombination as strategies for constructing very large phage libraries, Waterhouse et al., Nuc. Acids. Res., 21:2265-2266 (1993). Combinatorial antibodies are also discussed in Huse et al., Science 246:1275-1281 (1989), and Sastry et al., Proc. Natl. Acad. Sci. USA, 86:5728-5732 (1989), and Alting-Mees et al., Strategies in Molecular Biology 3:1-9 (1990). These references describe a system commercially available from Stratacyte, La Jolla, Calif. USA. Briefly, mRNA is isolated from a B cell population and utilized to create heavy and light chain immunoglobulin cDNA expression libraries in the γIMMUNOZAP(H) and γIMMUNOZAP(L) vectors. These vectors may be screened individually or co-expressed to form Fab fragments or antibodies, see Huse et al., supra; see also Sastry et al., supra. Positive plaques can subsequently be converted to a non-lytic plasmid, which allows for high-level expression of monoclonal antibody fragments from E. coli.

Humanized MOAB

Binding partners can also be constructed utilizing recombinant DNA techniques to incorporate the variable regions of a gene that encode a specifically binding antibody. The construction of these binding partners can be readily accomplished by one of ordinary skill in the art in view of the present application. See Larrick et al., Biotechnology, 7:934-938 (1989); Riechmann et al., Nature, 332:323-327 (1988); Roberts et al., Nature, 328:731-734 (1987); Verhoeyen et al., Science 239:1534-1536 (1988); Chaudhary et al., Nature, 339:394-397 (1989); see also U.S. Pat. No. 5,132,405 entitled “Biosynthetic Antibody Binding Sites”.) For example, the DNA can be modified by substituting the coding sequence for human heavy- and light-chain constant domains in place of homologous murine sequences, U.S. Pat. No. 4,816,567; Morrison, et al., Proc. Nat. Acad. Sci., 81:6851 (1984), or by covalently joining to the immunoglobulin coding sequence all or part of the coding sequence for a non-immunoglobulin polypeptide. In another example, DNA segments encoding the desired antigen-binding domains specific for the protein or peptide of interest are amplified from appropriate hybridomas and inserted directly into the genome of a cell that produces human antibodies. See Verhoeyen et al., supra; see also Reichmann et al., supra. Some of these techniques transfer the antigen-binding site of a specifically binding mouse or rat monoclonal antibody or the like to a human antibody. Such antibodies can be preferable for therapeutic use in humans because they are typically not as antigenic as rat or mouse antibodies.

In an alternative embodiment, genes that encode the variable region from a hybridoma producing a monoclonal antibody of interest can be amplified using oligonucleotide primers for the variable region. These primers may be synthesized by one of ordinary skill in the art, or may be purchased from commercially available sources. For instance, primers for mouse and human variable regions including, among others, primers for V_{H}a, V_{H}b, V_{H}c, V_{H}d, C_{H}1, V_{L}, and C_{L} regions are available from Stratacyte (La Jolla, Calif.). These primers may be utilized to amplify heavy- or light-chain variable regions, which may then be inserted into vectors such as IMMUNOZAP™ (H) or IMMUNOZAP™ (L) (Stratacyte), respectively. These vectors may then be introduced into E. coli for expression. Utilizing these techniques, large amounts of a single-chain protein containing a fusion of the V_{H} and V_{L} domains may be produced, see Bird et al., Science 242:423-426 (1988).

Antibody Substitutions—Non-Immunoglobulin Polypeptides (All Abs)

Non-immunoglobulin polypeptides can be substituted in monoclonal and other antibodies described herein for the constant domains of an antibody, or they can be substituted for the variable domains of one antigen-combining site of an antibody to create a chimeric bivalent antibody comprising one antigen-combining site having specificity for an antigen and another antigen-combining site having specificity for a different antigen.

Chimerics

Chimeric or hybrid antibodies can also be prepared in vitro using known methods in synthetic protein chemistry, including those involving crosslinking agents, in view of the present application. For example, immunotoxins may be constructed using a disulfide-exchange reaction or by forming a thioether bond. Examples of suitable reagents for this purpose include iminothiolate and methyl-4-mercaptobutyrimidate.

Antibody Labeling (All Abs)

For diagnostic applications or otherwise as desired, and for monoclonal and other antibodies described herein, the antibodies and other binding partners typically will be labeled with a detectable moiety. The detectable moiety can be any moiety that is capable of producing, either directly or indirectly, a detectable signal. For example, the detectable moiety may be a radioisotope, such as ³H, ⁴C, ³²P, ³⁵S, or ¹²⁵I; a fluorescent or chemiluminescent compound, such as fluorescein isothiocyanate, rhodamine, or luciferin; or an enzyme, such as alkaline phosphatase, beta-galactosidase, or horseradish peroxidase. Any method known in the art for conjugating the antibody or binding partner to the detectable moiety may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. Cytochem., 30:407 (1982).

(iii) Humanized And Human Antibodies

Humanized Ab Generally

Methods for humanizing non-human antibodies are well known in the art and have been discussed in part above. Generally, a humanized antibody has one or more amino acid residues introduced into it from a source which is non-human. These non-human amino acid residues are often referred to as “import” residues, which are typically taken from an “import” variable domain. Humanization can be performed essentially following the method of Winter and co-workers, Jones et al., Nature, 321:522-525 (1986); Riechmann et al., Nature, 332:323-327 (1988); Verhoeyen et al., Science, 239:1534-1536 (1988), by substituting rodent CDRs or CDR sequences for the corresponding sequences of a human antibody. Accordingly, such humanized antibodies are chimeric antibodies, U.S. Pat. No. 4,816,567, wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making humanized antibodies is very important to reduce antigenicity. According to the so-called “best-fit” method, the sequence of the variable domain of a rodent antibody is screened against the entire library of known human variable-domain sequences. The human sequence that is closest to that of the rodent is then accepted as the human framework (FR) for the humanized antibody. Sims et al., J. Immunol., 151:2296 (1993); Chothia and Lesk, J. Mol. Biol., 196:901 (1987). Another method uses a particular framework derived from the consensus sequence of all human antibodies of a particular subgroup of light or heavy chains. The same framework may be used for several different humanized antibodies. Carter et al., Proc. Natl. Acad. Sci. USA, 89:4285 (1992); Presta et al., J. Immunol., 151:2623 (1993).

It is typically desirable that antibodies be humanized with retention of high affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence, e.g., the analysis of residues that influence the ability of the candidate immunoglobulin to bind antigen. In this way, FR residues can be selected and combined from the consensus and import sequences so that the desired antibody characteristic, such as increased affinity for the target antigen(s), is achieved. In general, CDR residues are directly and most substantially involved in influencing antigen binding.

It is also possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA. 90:2551-255 (1993); Jakobovits et al., Nature, 362:255-258 (1993); Bruggemann et al., Year Immuno., 7:33 (1993). Human antibodies can also be produced in phage-display libraries, Hoogenboom and Winter, J. Mol. Biol., 227:381 (1991); Marks et al., J. Mol. Biol., 222:581 (1991).

(iv) Antibody Fragments

Antibody Fragments

Various techniques have been developed for the production of antibody fragments. Such fragments can be derived via proteolytic digestion of intact antibodies, see, e.g., Morimoto et al., J. Biochem. Biophys. Meth. 24:107-117 (1992) and Brennan et al., Science, 229:81 (1985). Fragments can also be produced directly by recombinant host cells. For example, antibody fragments can be isolated from antibody phage libraries discussed above. Fab′-SH fragments can be directly recovered from E. coli and chemically coupled to form F(ab′)₂ fragments, Carter et al., Biotechnology 10:163-167 (1992). F(ab′)₂ fragments can be isolated directly from recombinant host cell culture. Other techniques for the production of antibody fragments will be apparent to the skilled practitioner.

(v) Bispecific Antibodies

Bispecific Antibodies Generally

Bispecific antibodies (BsAbs) are antibodies that have binding specificities for at least two different antigens. Bispecific antibodies can be derived from full-length antibodies or from antibody fragments, e.g., F(ab′)₂ bispecific antibodies.

Methods for making bispecific antibodies are known in the art. Traditional production of full-length bispecific antibodies is based on the coexpression of two immunoglobulin heavy chain-light chain pairs, where the two chains have different specificities, Millstein and Cuello, Nature, 305:537-539 (1983). Because of the random assortment of immunoglobulin heavy and light chains, these hybridomas (quadromas) produce a mixture of potentially 10 different antibody molecules, of which only one has the correct bispecific structure. Purification of the correct molecule, which is usually accomplished by affinity chromatography steps, is rather cumbersome, and the product yields are low. Similar procedures are disclosed in WO 93/08829, and in Traunecker et al., E.M.B.O. J., 10:3655-3659 (1991).

According to another approach, antibody variable domains containing the desired binding specificities (antibody-antigen combining sites) are fused to immunoglobulin constant domain sequences. The fusion is preferably with an immunoglobulin heavy chain constant domain, comprising at least part of the hinge, C_{H} 2, and C_{H} 3 regions. It is preferred to have the first heavy-chain constant region (C_{H} 1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

Antibodies—Hybrid Immunoglobulin Heavy Chain

In one embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure may facilitate the separation of the desired bispecific compound from unwanted immunoglobulin chain combinations, as the presence of an immunoglobulin light chain in only one half of the bispecific molecule provides for a facile method of separation. This approach is disclosed in WO 94/04690. For further details of generating bispecific antibodies see, for example, Suresh et al., Meth. Enzymol., 121:210 (1986).

Antibodies—Cross-Linked or “Heteroconjugate”

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells, U.S. Pat. No. 4,676,980), and for treatment of HIV infection, WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Antibodies—Diabodies

The “diabody” technology described by Hollinger et al., Proc. Natl. Acad. Sci. USA, 90:6444-6448 (1993) has provided an alternative mechanism for making BsAb fragments. The fragments comprise a heavy-chain variable domain (V_{H}) connected to a light-chain variable domain (V_{L}) by a linker that is too short to allow pairing between the two domains on the same chain. Accordingly, the VH and VL domains of one fragment are forced to pair with the complementary V_{L} and V_{H} domains of another fragment, thereby forming two antigen-binding sites.

Another strategy for making BsAb fragments by the use of single-chain Fv (sFv) dimers has also been reported. See Gruber et al., J. Immunol., 152:5368 (1994). These researchers designed an antibody comprising the V_{H} and V_{L} domains of a first antibody joined by a 25-amino-acid-residue linker to the V_{H} and V_{L} domains of a second antibody. The refolded molecule bound to fluorescein and the T-cell receptor and redirected the lysis of human tumor cells that had fluorescein covalently linked to their surface.

Antibodies—Other

Techniques for generating bispecific antibodies from antibody fragments have also been described in the literature. For example, bispecific antibodies can be prepared using chemical linkage. Brennan et al., Science, 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the BsAb. The BsAbs produced can be used as agents for the selective immobilization of enzymes.

Fab′-SH fragments can be directly recovered from E. coli, which can be chemically coupled to form bispecific antibodies. Shalaby et al., J. Exp. Med., 175:217-225 (1992) describe the production of a fully humanized BsAb F(ab′)₂ molecule. Each Fab′ fragment was separately secreted from E. coli and subjected to directed chemical coupling in vitro to form the BsAb. The BsAb thus formed was able to bind to cells overexpressing the HER2 receptor and normal human T cells, as well as trigger the lytic activity of human cytotoxic lymphocytes against human breast tumor targets. See also Rodriguez et al., Int. J. Cancers (Suppl.) 7:45-50 (1992).

Various techniques for making and isolating BsAb fragments directly from recombinant cell culture have also been described. For example, bispecific F(ab′)₂ heterodimers have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers.

b. Antibody Purification

Antibody Purification Generally

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., supra, Bio/Technology 10:163-167 (1992), describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

Before LPHIC

The antibody composition prepared from the cells is preferably subjected to at least one purification step prior to LPHIC. Examples of suitable purification steps include hydroxyapatite chromatography, gel electrophoresis, dialysis, and affinity chromatography. The suitability of protein A as an affinity ligand depends on the species and isotype of any immunoglobulin Fe domain that is present in the antibody. Protein A can be used to purify antibodies that are based on human γ1, γ2, or γ4 heavy chains, Lindmark et al., J. Immunol. Meth. 62:1-13 (1983). Protein G has been recommended for mouse isotypes and for human γ3, Guss et al., E.M.B.O. J., 5:1567-1575 (1986). The matrix to which the affinity ligand is attached is often agarose, but other matrices are available. Mechanically stable matrices such as controlled pore glass or poly(styrenedivinyl)benzene allow for faster flow rates and shorter processing times than can be achieved with agarose. Where the antibody comprises a C_{H} 3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™, chromatography on an anion or cation exchange resin (such as a polyaspartic acid column), chromatofocusing, SDS-PAGE, and ammonium sulfate precipitation are also available depending on the antibody to be recovered.

LPHIC

Following any preliminary purification step(s), the mixture comprising the antibody of interest and contaminant(s) can be subjected to LPHIC. See U.S. Pat. No. 6,214,984. Often, the antibody composition to be purified will be present in a buffer from the previous purification step. However, it may be necessary to add a buffer to the antibody composition prior to the LPHIC step. Many buffers are available and can be selected by routine experimentation. The pH of the mixture comprising the antibody to be purified and at least one contaminant in a loading buffer is adjusted to a pH of about 2.5-4.5 using either an acid or base, depending on the starting pH. The loading buffer can have a low salt concentration (e.g., less than about 0.25 M salt).

The mixture is loaded on the HIC column. HIC columns normally comprise a base matrix (e.g., cross-linked agarose or synthetic copolymer material) to which hydrophobic ligands (e.g., alkyl or aryl groups) are coupled. One example of an HIC column comprises an agarose resin substituted with phenyl groups (e.g., a Phenyl SEPHAROSETM column). Many HIC columns are available commercially. Examples include, but are not limited to, Phenyl SEPHAROSE 6 FAST FLOW™ column with low or high substitution (Pharmacia LKB Biotechnology, AB, Sweden); Phenyl SEPHAROSE™ High Performance column (Pharmacia LKB Biotechnology, AB, Sweden); Octyl SEPHAROSE™ High Performance column (Pharmacia LKB Biotechnology, AB, Sweden); FRACTOGEL™ EMD Propyl or FRACTOGEL™ EMD Phenyl columns (E. Merck, Germany); MACRO-PREP™ Methyl or MACRO-PREP™ t-Butyl Supports (Bio-Rad, California); WP HI-Propyl (C₃)™ column (J. T. Baker, New Jersey); and TOYOPEARL™ ether, phenyl or butyl columns (TosoHaas, Pa.).

The antibody is typically eluted from the column using an elution buffer that is the same as the loading buffer. The elution buffer can be selected using routine experimentation in view of the present application. The pH of the elution buffer is between about 2.5-4.5 and has a low salt concentration (e.g., less than about 0.25 M salt). It may not be necessary to use a salt gradient to elute the antibody of interest; the desired product may be recovered in the flow-through fraction that does not bind significantly to the column.

he LPHIC step provides a way to remove a correctly folded and disulfide bonded antibody from unwanted contaminants (e.g., incorrectly associated light and heavy fragments). The method can provide an approach to substantially remove an impurity characterized as a correctly folded antibody fragment whose light and heavy chains fail to associate through disulfide bonding. Antibody compositions prepared using LPHIC can be up to about 95% pure or more. Purities of more than about 98% have been reported. U.S. Pat. No. 6,214,984.

Post LPHIC

Antibody compositions prepared by LPHIC can be further purified as desired using techniques which are well known in the art. Diagnostic or therapeutic formulations of the purified protein can be made by providing the antibody composition in a physiologically acceptable carrier, examples of which are provided below. To remove contaminants (e.g., unfolded antibody and incorrectly associated light and heavy fragments) from the HIC column so that it can be re-used, a composition including urea (e.g., 6.0 M urea, 1% MES buffer pH 6.0, 4 mM ammonium sulfate) can be flowed through the column.

c. Some Uses For Antibodies Described Herein

(i) Generally

Generally

The present invention comprises any suitable use for the antibodies and other binding partners discussed herein. The following provides some of the desired uses, including diagnostic and therapeutic uses. Various diagnostic and therapeutic uses for antibodies have been reviewed in Goldenberg et al., Semin. Cancer Biol., 1(3):217-225 (1990); Beck et al., Semin. Cancer Biol., 1(3):181-188 (1990); Niman, Immunol. Ser. 53:189-204 (1990); and, Endo, Nippon Igaku Hoshasen Gakkai Zasshi (Japan) 50(8):901-909 (1990), for example.

Assays

The antibodies can be used in immunoassays, such as enzyme immunoassays. BsAbs can be useful for this type of assay; one arm of the BsAb can be designed to bind to a specific epitope on the enzyme so that binding does not cause enzyme inhibition, the other arm of the antibody can be designed to bind to an immobilizing matrix ensuring a high enzyme density at the desired site. Examples of such diagnostic BsAbs include those having specificity for IgG as well as ferritin, and those having binding specificities for horseradish peroxidase (HRP) as well as a hormone, for example. Monoclonal and polyclonal antibodies are also exemplary antibodies for immunoassays.

The antibodies can be designed for use in two-site immunoassays. For example, two antibodies are produced binding to two separate epitopes on the analyte protein; one antibody binds the complex to an insoluble matrix, the other binds an indicator enzyme.

Diagnostic Uses

Antibodies can also be used for immunodiagnosis, in vitro or in vivo or otherwise, of various diseases or conditions based on the presence or absence of GPR 38. Such diseases and conditions include Alzheimer's disease and Parkinson's disease, inflammatory bowel diseases including ulcerative colitis and Crohn's disease, Hodgkin's disease, glioblastoma and carcinomas including breast, colon, lung (small cell and adenocarcinoma), pancreatic (small cell and adenocarcinoma), ovarian, and prostate. To facilitate this diagnostic use, an antibody that binds an antigen such as GPR 38, which is differentially expressed in tumors, can be conjugated with a detectable marker (e.g., a chelator that binds a radionuclide). Examples of other tumor-associated antigens being used in a similar fashion include an antibody having specificity for the tumor-associated antigen CEA used for imaging colorectal and thyroid carcinomas and the anti-p185^{HER2} antibody used for detecting cancers characterized by amplification of the HER2 protooncogene. Other uses for the antibodies of the present invention will be apparent to the skilled practitioner in view of the present application.

(ii) Assays

Assays

For certain applications such as some diagnostic and other assay applications, the antibody typically can be labeled directly or indirectly with a detectable moiety. The detectable moiety can be any moiety that is capable of producing, either directly or indirectly, a detectable signal. For example, the detectable moiety may be a radioisotope, such as ³H, ¹⁴C, ³²P, ³⁵S, or 125I; a fluorescent or chemiluminescent compound, such as fluorescein isothiocyanate, rhodamine, or luciferin; or an enzyme, such as alkaline phosphatase, beta-galactosidase or HRP.

Any method known in the art for separately conjugating the antibody to the detectable moiety may be employed, including those methods described by Hunter et al., Nature, 144:945 (1962); David et al., Biochemistry, 13:1014 (1974); Pain et al., J. Immunol. Meth. 40:219 (1981); and, Nygren, J. Histochem. and Cytochem. 30:407 (1982).

The antibodies of the present invention may be employed in any desired assay method, such as competitive binding assays, direct and indirect sandwich assays, and immunoprecipitation assays. Zola, Monoclonal Antibodies: A Manual of Techniques, pp. 147-158 (CRC Press, Inc. (1987).

Competitive Binding Assays

Competitive binding assays rely on the ability of a labeled standard to compete with the test sample analyte for binding with a limited amount of antibody. The amount of analyte in the test sample is inversely proportional to the amount of standard that becomes bound to the antibody. To facilitate determining the amount of standard that becomes bound, the antibody generally is insolubilized before or after the competition, so that the standard and analyte that are bound to the antibody may conveniently be separated from the standard and analyte which remain unbound.

BsAbs are particularly useful for sandwich assays which involve the use of two molecules, each capable of binding to a different immunogenic portion, or epitope, of the sample to be detected. In a sandwich assay, the test sample analyte is bound by a first arm of the antibody which is immobilized on a solid support, and thereafter a second arm of the antibody binds to the analyte, thus forming an insoluble three part complex. See, e.g., U.S. Pat. No. 4,376,110. The second arm of the antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme. Assays are discussed further elsewhere herein in relation to binding partners such as antibodies, GPR 38 polypeptides, and polynucleotides, including assays searching for or using such GPR 38 polypeptides and polynucleotides, and would be apparent to those skilled in the art in view of the present application.

(iii) Affinity Purification

Affinity Purification

The antibodies also are useful for the affinity purification of an antigen of interest from recombinant cell culture or natural sources.

(iv) Therapeutics

Therapeutic Uses

Therapeutic compositions and uses, etc., for the antibodies described herein will now be discussed. As with other parts of this application, this section does not contain the entire discussion of therapeutic uses or compositions, etc., for antibodies; other sections discuss both antibodies and therapeutics, and the discussion in this section applies to certain other aspects discussed herein. Turning to antibodies and therapeutics, the antibodies can be used, for example, for targeting degenerating neurons, for redirected cytotoxicity (e.g., to kill tumor cells), for delivering immunotoxins to tumor cells, for converting enzyme activated prodrugs at a target site (e.g., a tumor), and for treating infectious diseases or targeting immune complexes to cell surface receptors.

Therapeutic Formulations

Therapeutic formulations of the antibody can be prepared for storage by mixing the antibody having the desired degree of purity with optional physiologically acceptable carriers, excipients, or stabilizers (Remington's Pharmaceutical Sciences, 16th edition, Osol, A., Ed. (1980), for example in the form of lyophilized cake or aqueous solutions. Acceptable carriers, excipients or stabilizers are nontoxic to recipients at the dosages and concentrations employed, and include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and nonionic surfactants such as Tween, Pluronics or polyethylene glycol (PEG).

The antibodies also may be entrapped in microcapsules prepared, for example, by coacervation techniques or by interfacial polymerization (for example, hydroxymethylcellulose or gelatin-microcapsules and poly-[methylmethacrylate] microcapsules, respectively), in colloidal drug delivery systems (for example, liposomes, albumin microspheres, microemulsions, nano-particles, and nanocapsules), or in macroemulsions. Such techniques are disclosed in Remington's Pharmaceutical Sciences, supra.

Therapeutic Formulations—Sterile

An antibody to be used for in vivo human administration should be sterile. This can be accomplished by filtration through sterile filtration membranes, for example prior to or following lyophilization and reconstitution. The antibody ordinarily will be stored in lyophilized form or in solution. Therapeutic antibody compositions generally are placed into a container having a sterile access port, for example, an intravenous solution bag or vial having a stopper pierceable by a hypodermic injection needle.

Therapeutic Administrations

The route of antibody administration is in accord with known methods, e.g., injection or infusion by intravenous, intraperitoneal, intracerebral, intramuscular, intraocular, intraarterial, or intralesional routes, or by sustained release systems as noted below.

The antibody can be administered, for example, continuously by infusion or by bolus injection. Suitable examples of sustained-release preparations include semipermeable matrices of solid hydrophobic polymers containing the protein, which matrices are in the form of shaped articles, e.g., films, or microcapsules. Examples of sustained-release matrices include polyesters, hydrogels (e.g., poly(2-hydroxyethyl-methacrylate) as described by Langer et al., J. Biomed. Mater. Res., 15:167-277 (1981), and Langer, Chem. Tech., 12:98-105 (1982), or poly(vinylalcohol)), polylactides, U.S. Pat. No. 3,773,919; EP 58,481, copolymers of L-glutamic acid and gamma ethyl-L-glutamate, Sidman et al., Biopolymers, 22:547-556 (1983), non-degradable ethylene-vinyl acetate, Langer et al., supra, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid, EP 133,988.

Therapeutic Administrations—Sustained Release—Polymers

While polymers such as ethylene-vinyl acetate and lactic acid-glycolic acid sustain release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for antibody stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Therapeutic Administrations—Sustained Release-Liposomes

Sustained-release antibody compositions also include liposomally entrapped antibody. Liposomes containing the antibody can be prepared by methods such as those in DE 3,218,121; Epstein et al., Proc. Natl. Acad. Sci. USA, 82:3688-3692 (1985); Hwang et al., Proc. Natl. Acad. Sci. USA, 77:4030-4034 (1980); EP 52,322; EP 36,676; EP 88,046; EP 143,949; EP 142,641; Japanese patent application 83-118008; U.S. Pat. Nos. 4,485,045 and 4,544,545; and EP 102,324. Ordinarily the liposomes are of the small (about 200-800 Angstroms) unilamellar type in which the lipid content is greater than about 30 mol. % cholesterol, the selected proportion being adjusted for the optimal antibody therapy.

Therapeutically Effective Amount

An effective amount of antibody to be employed therapeutically will depend, for example, upon therapeutic objectives, the route of administration, and the condition of the patient. Accordingly, it will be necessary for therapist to titer the dosage and modify the route of administration as required to obtain the optimal therapeutic effect. A typical daily dosage might range from about 1 μg/kg to up to 10 mg/kg or more, depending on the factors mentioned above. Typically, the clinician will administer antibody until a dosage is reached that achieves the desired effect. The progress of this therapy is easily monitored by conventional assays.

5. Drug Design Based on GPR 38

Use of GPR 38 for Drug Design

GPR 38 can serve as a valuable tool for designing drugs for treating various pathophysiological conditions such as Alzheimer's disease and Parkinson's disease, inflammatory bowel diseases including ulcerative colitis and Crohn's disease, Hodgkin's disease, glioblastoma, and carcinomas including breast, colon, lung (small cell and adenocarcinoma), pancreatic (small cell and adenocarcinoma), ovarian, and prostate among others and diagnostic assays for such conditions, as well as other diseases or conditions as described herein or that would be readily apparent to those skilled in the art in view of the present application.

6. Therapeutics Related to GPR 38

a. Generally

Compositions—Carriers, Adjuvants, etc.

For administration to a patient, one or more polypeptides, polynucleotides, antibodies, modulating agents, etc., as described herein are generally formulated as a pharmaceutical composition, which may be a sterile aqueous or non-aqueous solution, suspension or emulsion, and which additionally comprises a physiologically acceptable carrier (e.g., a non-toxic material that does not interfere with the activity of the active ingredient), binder, excipient, buffer, adjuvant, dispersion agent, or other desired element. Any suitable carrier, etc., known to those of ordinary skill in the art may be employed in a pharmaceutical composition. Representative carriers include physiological saline solutions, gelatin, water, alcohols, natural or synthetic oils, saccharide solutions, glycols, injectable organic esters such as ethyl oleate or a combination of such materials. Such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, antimicrobial compounds, chelating agents such as EDTA or glutathione, adjuvants (e.g., aluminum hydroxide), inert gases, or preservatives. Compositions of the present invention may also be formulated as a lyophilizate. Pharmaceutical compositions may also contain other compounds, which may be biologically or therapeutically active or inactive.

Sustained Release

The compositions described herein may be administered as part of a sustained release formulation (e.g., a formulation such as a capsule that effects a slow release of compound following administration). Such formulations may generally be prepared using well known technology and administered by, for example, oral, rectal, or subcutaneous implantation, or by implantation at the desired target site. Sustained-release formulations may contain a polypeptide, polynucleotide or modulating agent dispersed in a carrier matrix or contained within a reservoir surrounded by a rate controlling membrane. Carriers for use within such formulations are biocompatible, and may also be biodegradable; preferably the formulation provides a relatively constant level of release. The amount of active compound contained within a sustained release formulation depends upon the site of implantation, the rate and expected duration of release, and the nature of the condition to be treated or prevented.

Therapeutic Applications

The polypeptides, polynucleotides, modulating agents, agonists, antagonists, etc., herein may be used to provide various therapies and medicaments, including processed for making medicaments, related to the motilin binding or secondary messenger actions of GPR 38; some of these applications are discussed elsewhere herein, or would be apparent to those skilled in the art in view of the present application. Briefly, the motilin binding or secondary messenger actions of GPR 38 are implicated in Alzheimer's disease, Parkinson's disease, inflammatory bowel diseases including ulcerative colitis and Crohn's disease, Hodgkin's disease, glioblastoma, and carcinomas including breast, colon, lung (small cell and adenocarcinoma), pancreatic (small cell and adenocarcinoma), ovarian, and prostate. Thus, the present invention provides for remediation or inhibition of such diseases based on GPR 38 in a patient. A “patient” may be any mammal, preferably a human, and may be afflicted with the disease, or may be free of detectable disease. Accordingly, the treatment may be of an existing disease or may be prophylactic.

Modulating Biological Activity

Treatment includes administration of a composition or compound which modulates the biological activity of GPR 38. Such modulation includes the suppression of GPR 38 expression or activity when it is over-expressed, or augmentation of GPR 38 expression or activity when it is under-expressed. Modulation can also include the suppression or enhancement of motilin binding by GPR 38, or the biological activity of motilin related to Alzheimer's disease and Parkinson's disease, inflammatory bowel diseases including ulcerative colitis and Crohn's disease, Hodgkin's disease, glioblastoma, and breast carcinoma, colon carcinoma, lung carcinoma (small cell and adenocarcinoma), pancreatic carcinoma (small cell and adenocarcinoma), ovarian carcinoma, and prostate carcinoma. In general, for administration to a patient, an antibody or other agent is formulated as a pharmaceutical composition as described herein. A suitable dose of such an agent is an amount sufficient to show benefit in the patient based on the criteria noted herein.

Routes of Administration

Therapeutic agents can be provided as a liquid solution, or as a solid form (e.g., lyophilized) which can be resuspended in a solution prior to administration. Therapeutic agents can be typically administered via traditional direct routes, such as buccal/sublingual, rectal, oral, nasal, topical (such as transdermal and ophthalmic), vaginal, pulmonary, intracranial, intraarterial, intramuscular, intraperitoneal, subcutaneous, intraocular, intranasal or intravenous, or via indirect routes. Non-parenteral routes are discussed further in. See WO 96/20732.

b. Discussion Directed Primarily to Polypeptides

Dosage Regimens

The GPCR agonists, antagonists, and other polypeptide-based therapeutic agents of this invention can be used therapeutically to stimulate or inhibit depending on the desired result the activity of GPR 38, for example via the action of a specific ligand, or the endogenous ligand, for GPR 38, and thereby to treat medical conditions and situations caused by, mediated by or otherwise related to specific or endogenous ligand, or otherwise to improve or enhance a medical condition by providing a desired biological activity. As with other therapeutic regimens for the present application, the dosage regimen involved in a therapeutic application will be determined by the attending physician, considering various factors that may modify the action of therapeutic substance, e.g., the condition, body weight, sex, and diet of the patient, the severity of any infection or other condition, including complicating conditions, time of administration, and other clinical factors.

Administration Protocols

Typical protocols for therapeutic administration of such substances can be determined by a person skilled in the art in view of the present application. Administration of the compositions can be any desired route including those described herein such as parenteral (e.g., intraperitoneal, intravenous, subcutaneous, or intramuscular injection), non-parenteral, or by infusion or by any other acceptable systemic or local method as desired. Often, treatment dosages are titrated upward from a low level to optimize safety and efficacy. Generally, daily dosages will fall within a range of about 0.01 to 20 mg protein per kilogram of body weight. Typically, the dosage range will be from about 0.1 to 5 mg per kilogram of body weight. Dosages can be adjusted to account for variations in molecular size and half-life (clearance times) following administration. An “effective amount” of a composition of the invention is an amount that will ameliorate one or more of the well known parameters that characterize medical conditions caused or mediated by, or otherwise related to, specific or endogenous ligand.

The ligand agonists and antagonists of the invention encompass neutralizing antibodies or binding fragments thereof in addition to other types of inhibitors, including small organic molecules and inhibitory ligand analogs, which can be identified using the methods of the invention.

Pharmaceutical Additives (Carriers, Adjuvants, Buffering Agents, Dispersing Agents

The compositions can be administered in simple solution, or in combination with other materials such as carriers, preferably pharmaceutical carriers. Useful pharmaceutically acceptable carriers for nucleic acid-based therapeutic agents can often be useful for agonists and antagonists and other polypeptide agents discussed herein, provided appropriate desirable qualities are provided. Suitable carriers include any compatible, non-toxic substances suitable for delivering the compositions of the invention to a patient. Sterile water, alcohol, fats, waxes, and inert solids may be included in a carrier. Pharmaceutically acceptable adjuvants, including human-acceptable adjuvants selected from those discussed elsewhere herein, buffering agents or dispersing agents can also be incorporated into the pharmaceutical composition. Generally, compositions useful for parenteral administration of such drugs are well known; e.g., Remington's Pharmaceutical Science, 17th Ed., Mack Publishing Company, Easton, Pa. (1990). Alternatively, compositions of the invention may be introduced into a patient's body by implantable drug delivery systems, Urquhart et al., Ann. Rev. Pharmacol. Toxicol. 24:199 (1984).

Therapeutic formulations can be administered in many conventional dosage formulations. Formulations typically comprise at least one active ingredient, together with one or more pharmaceutically acceptable carriers. Formulations may include those suitable for oral, rectal, nasal, or parenteral (including subcutaneous, intramuscular, intravenous, and intradermal) administration.

The formulations may conveniently be presented in unit dosage form and may be prepared by any methods well known in the art of pharmacy. See, e.g., Gilman et al. (eds.) (1990), The Pharmacological Bases of Therapeutics, 8th Ed., Pergamon Press; and Remington's Pharmaceutical Sciences, supra, Easton, Pa.; Avis et al. (eds.) (1993) Pharmaceutical Dosage Forms: Parenteral Medications Dekker, New York; Lieberman et al. (eds.) (1990) Pharmaceutical Dosage Forms: Tablets Dekker, New York; and Lieberman et al. (eds.) (1990), Pharmaceutical Dosage Forms: Disperse Systems Dekker, New York.

c. Discussion Directed Primarily To Polynucleotides

General

Certain pharmaceutical compositions contain DNA or other polynucleotides encoding a polypeptide, antibody fragment or other modulating agent as described above (such that a GPR 38 polypeptide, or analog thereof, or a modulating agent is generated in situ) or an antisense polynucleotide. As indicated above and elsewhere herein, pharmaceutically acceptable carriers for nucleic acid-based therapeutic agents can often be useful for agonists, antagonists, and other polypeptides and other agents discussed herein, and vice-versa, provided appropriate desirable qualities are obtained. In such pharmaceutical compositions, the DNA may be present within any of a variety of delivery systems known to those of ordinary skill in the art, including nucleic acid, bacterial, and viral expression systems, as well as colloidal dispersion systems, or liposomes.

The GPCR agonists, antagonists, and other polypeptide-based therapeutic agents of this invention can be used therapeutically to stimulate or inhibit depending on the desired result the activity of GPR 38, for example via the action of a specific or endogenous ligand for GPR 38, and thereby to treat medical conditions and situations caused by, mediated by or otherwise related to the ligand, or otherwise to improve or enhance a medical condition by providing a desired biological activity. As with other therapeutic regimens for the present application, the dosage regimen involved in a therapeutic application will be determined by the attending physician, considering various factors that may modify the action of therapeutic substance, e.g., the condition, body weight, sex, and diet of the patient, the severity of any infection or other condition, including complicating conditions, time of administration, and other clinical factors.

Appropriate nucleic acid expression systems contain the necessary DNA sequences for expression in the patient (such as a suitable promoter and terminating signal). Such gene delivery vehicles (GDV) are also discussed elsewhere herein.

Carriers and Diluents

Pharmaceutically acceptable carriers or diluents, excipients, buffers, adjuvants, and the like are nontoxic to recipients at the dosages and concentrations employed. Representative examples of carriers or diluents for injectable solutions include water, isotonic saline solutions which are preferably buffered at a physiological pH (such as phosphate-buffered saline or Tris-buffered saline), mannitol, dextrose, glycerol, and ethanol, as well as polypeptides or proteins such as human serum albumin. In one exemplary composition where therapeutic agent comprises a GDV, such as a vector or recombinant virus carrying an antisense, gene therapy or ribozyme agent, the GDV can be provided in 10 mg/ml mannitol, 1 mg/ml HSA, 20 mM Tris, pH 7.2, and 150 mM NaCl. In such a composition, the GDV can represent approximately 1 μg of material, it may be less than 1% of high molecular weight material, and less than 1/100,000 of the total material (including water). Such compositions can be stable at −70° C. for at least six months.

Antisense

The present invention also encompasses anti-sense oligonucleotides capable of specifically hybridizing to mRNA encoding a GPR 38 or analogs and the like thereof so as to prevent translation of the mRNA. Based upon GPR 38 coding sequence, an antisense sequence is designed and preferably inserted into a vector suitable for transfection into host cells and expression of the antisense. The antisense nucleic acids should anneal to GPR 38 mRNA under physiological conditions. Preferably, the antisense does not anneal to other mRNAs, especially those of related molecules. Such antisense effectors may be produced by a variety of methods known in the art, including the use of a heterologous expression cassette introduced into cells. Such effectors and methods related thereto are described in detail in Antisense RNA and DNA (1988), D. A. Melton, Ed., Cold Spring Harbor Press, Cold Spring Harbor, N.Y.; U.S. Pat. No. 5,610,288; U.S. Pat. No. 5,665,580; and U.S. Pat. No. 5,681,944.

This invention further provides pharmaceutical compositions comprising (a) an amount of an oligonucleotide effective to reduce activity of GPR 38 by passing through a cell membrane and binding specifically with mRNA encoding GPR 38 in the cell so as to prevent its translation and (b) a pharmaceutically acceptable carrier capable of passing through a cell membrane. In one embodiment, the oligonucleotide is coupled to a substance that inactivates mRNA. In another embodiment, the substance that inactivates mRNA is a ribozyme; ribozymes are discussed further elsewhere herein.

Ribozymes

mRNA are RNA molecules that contain anti-sense sequences for GPR 38 and an RNA-cleaving enzymatic activity that cleaves a specific site in a target RNA. Two types of ribozymes are the hammerhead ribozyme, Rossi, J. J., et al., Pharmac. Ther., 50:245-254 (1991) and the hairpin ribozyme, Hampel et al., Nucl. Acids Res., 18:299-304 (1990), and U.S. Pat. No. 5,254,678. The recognition sequences for hairpin ribozymes and for hammerhead ribozymes are known. The additional nucleotides of the hammerhead ribozyme or hairpin ribozyme are determined by the target flanking nucleotides and the hammerhead consensus sequence, see Ruffner et al., Biochemistry, 29:10695-10702 (1990). The preparation and use of certain ribozymes is described in U.S. Pat. No. 4,987,071. Ribozymes can be expressed from a vector introduced into the host cells.

Gene Therapy

GPR 38 polypeptides, such as antagonists or agonists or other agents that are polypeptides, can be employed by expression of such polypeptides in vivo, which is often referred to as “gene therapy.”

For example, cells from a patient may be engineered with a polynucleotide (DNA or RNA) encoding a polypeptide ex vivo, with the engineered cells then being provided to a patient to be treated with the polypeptide. Such methods are well known in the art. For example, cells may be engineered by procedures known in the art by use of a retroviral particle containing RNA encoding a polypeptide of the present invention.

Similarly, cells may be engineered in vivo for expression of a polypeptide in vivo by, for example, procedures known in the art. As known in the art, a producer cell for producing a retroviral particle containing RNA encoding the polypeptide of the present invention may be administered to a patient for engineering cells in vivo and expression of the polypeptide in vivo. These and other methods for administering a polypeptide of the present invention by such method should be apparent to those skilled in the art from the teachings of the present invention. For example, the expression vehicle for engineering cells may be other than a retrovirus, for example, an adenovirus which may be used to engineer cells in vivo after combination with a suitable delivery vehicle.

Retroviruses from which the retroviral plasmid vectors hereinabove mentioned may be derived include, but are not limited to, Moloney Murine Leukemia Virus, spleen necrosis virus, retroviruses such as Rous Sarcoma Virus, Harvey Sarcoma Virus, avian leukosis virus, gibbon ape leukemia virus, human immunodeficiency virus, adenovirus, Myeloproliferative Sarcoma Virus, and mammary tumor virus.

The vector includes one or more promoters. Suitable promoters include the retroviral LTR; the SV40 promoter; and, the human cytomegalovirus (CMV) promoter described in Miller, et al., Biotechniques, Vol. 7, No. 9, 980-990 (1989), or any other promoter (e.g., cellular promoters such as eukaryotic cellular promoters including the histone, pol III, and β-actin promoters). Other viral promoters include adenovirus promoters, thymidine kinase (TK) promoters, and B19 parvovirus promoters. The selection of a suitable promoter will be apparent to those skilled in the art in view of the present application.

The nucleic acid sequence encoding the polypeptide of the present invention is under the control of a suitable promoter. Suitable promoters include adenoviral promoters, such as the adenoviral major late promoter; or hetorologous promoters, such as the cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTRs (including the modified retroviral LTRs hereinabove described); the β-actin promoter; and human growth hormone promoters. The promoter also may be the native promoter which controls the gene encoding the polypeptide.

The retroviral plasmid vector is employed to transduce packaging cell lines to form producer cell lines. Examples of packaging cells which may be transfected include, but are not limited to, the PE501, PA317, ψ-2, ψ-AM, PA12, T19-14X, VT-19-17-H2, ψCRE, ψCRIP, GP+E-86, GP+envAm12, and DAN cell lines as described in Miller, Human Gene Therapy, Vol. 1, pgs. 5-14 (1990). The vector may transduce the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host.

The producer cell line generates infectious retroviral vector particles which include the nucleic acid sequence(s) encoding the polypeptides. Such retroviral vector particles then may be employed, to transduce eukaryotic cells, either in vitro or in vivo. The transduced eukaryotic cells will express the nucleic acid sequence(s) encoding the polypeptide. Eukaryotic cells which may be transduced include, but are not limited to, embryonic stem cells, embryonic carcinoma cells, as well as hematopoietic stem cells, hepatocytes, fibroblasts, myoblasts, keratinocytes, endothelial cells, and bronchial epithelial cells.

Vectors Generally—GDV

Turning to a general discussion of vectors that are useful in accordance with the present invention, including some of those discussed elsewhere herein, a “gene delivery vehicle” is a recombinant vehicle, such as a viral vector, a nucleic acid vector (such as plasmid), a naked nucleic acid molecule such as a gene, a retrotransposon, a cosmid, a nucleic acid molecule complexed to a polycationic molecule capable of neutralizing the negative charge on the nucleic acid molecule and condensing the nucleic acid molecule into a compact molecule, a bacterium, and certain eukaryotic cells such as a producer cell, that are capable of delivering a nucleic acid molecule having one or more desirable properties to host cells in an organism. See WO 96/20731A; WO 96/21015; WO 96/20732.

Typically, the GDV is an assembly that carries a nucleic acid molecule (or sequence), such molecule often capable of expressing sequences or genes of interest. In the context of protein expression, the GDV typically includes promoter elements such as for RNA Polymerase II or RNA replicase, and may include a signal that directs polyadenylation. In addition, the GDV preferably includes a molecule that, when transcribed, is operably linked to the molecules or genes of interest and acts as a translation initiation sequence. The GDV may include a selectable marker such as neomycin, thymidine kinase, hygromycin, phleomycin, histidinol, or dihydrofolate reductase (DHFR), as well as one or more restriction sites and a translation termination sequence. In addition, if the GDV comprises a retroviral particle, the GDV must include a retroviral packaging signal and LTRs appropriate to the retrovirus used, provided these are not already present. The GDV can also be used in combination with other viral vectors or inserted physically into cells or tissues as described below. The GDV may include a sequence that encodes a protein or active portion of the protein, antisense or ribozyme. Such sequences may be designed to inhibit MHC antigen presentation in order to suppress the immune response of cytotoxic T-lymphocytes against a transplanted tissue.

GDV—Viral Vectors

Viral vectors useful as a GDV include recombinant retroviral vectors and recombinant adenovirus vectors. The construction of recombinant retroviral vectors is described in U.S. Pat. Nos. 5,591,624; 5,716,832; 5,716,832; 5,716,613. Recombinant retroviral vectors may be used to generate transduction competent retroviral vector particles by introducing them into appropriate packaging cell lines, see U.S. Pat. Nos. 5,591,624; 5,716,832; 5,716,832; 5,716,613. Similarly, adenovirus vectors may also be readily prepared and utilized in view of the present application. See also Berkner, Biotechniques, 6:616-627 (1988), and Rosenfeld et al., Science, 252:431-434 (1991), WO 93/07283, WO 93/06223, and WO 93/07282).

The GDV can be a Sindbis RNA expression vector that includes, in order, a 5′ sequence which is capable of initiating transcription of a Sindbis virus, a nucleotide sequence encoding Sindbis non-structural proteins, a viral junction region, a heterologous sequence, a Sindbis RNA polymerase recognition sequence, and a stretch of 25 consecutive polyadenylate residues. A wide variety of heterologous sequences may be included in the GDV. Within various embodiments of the invention, the GDV may contain (and express, within certain embodiments) two or more heterologous sequences.

Other viral vectors suitable for use in the present invention include, for example, poliovirus, Evans et al., Nature, 339:385-388 (1989), and Sabin, J. of Biol., Standardization 1:115-118 (1973); rhinovirus, Arnold, J. Cell. Biochem. L401-405 (1990); pox viruses, such as canary pox virus or vaccinia virus, Fisher-Hoch et al., PNAS 86:317-321 (1989); Flexner et al., Ann. N.Y. Acad. Sci. 569:86-103 (1989); Flexner et al., Vaccine 8:17-21 (1990); U.S. Pat. Nos. 4,603,112 and 4,769,330; WO 89/01973); SV40, Mulligan et al., Nature, 277:108-114 (1979); influenza virus, Luytjes et al., Cell, 59:1107-1113 (1989); McMicheal et al., The New England Journal of Medicine 309:13-17 (1983); and Yap et al., Nature, 273:238-239 (1978); parvovirus such as adeno-associated virus, Samulski et al., Journal of Virology 63:3822-3828 (1989), and Mendelson et al., Virology 166:154-165 (1988); herpes, Kit, Adv. Exp. Med. Biol., 215:219-236 (1989); HIV; measles, EP 0,440,219); measles, EP 0 440,219); astrovirus, Munroe, S. S. et al., J. Vir., 67:3611-3614 (1993); Semliki Forest Virus, and coronavirus, as well as other viral systems, e.g., EP 0,440,219; WO 92/06693; U.S. Pat. No. 5,166,057). In addition, viral carriers may be homologous, non-pathogenic (defective), replication competent virus, e.g., Overbaugh et al., Science 239:906-910 (1988).

here the GDV is a retroviral vector, the nucleic acid molecules carried by the retroviral vector are typically of a size sufficient to allow production of viable virus. The production of any measurable titer of infectious virus on susceptible monolayers is considered to be “production of viable virus.” Within preferred embodiments, a heterologous sequence within the retroviral vector GDV will comprise at least 100 bases, at least 2 kb, 3.5 kb, 5 kb, or 7 kb, or even a heterologous sequence of at least 8 kb.

GDV—Naked Vectors

A nucleic acid molecule without any covering, such as a viral capsid or bacterial cell membrane, is also suitable for use as a GDV within the present invention. See Ulmer et al., Science 259:1745-1749 (1993). Such “naked” nucleic acids include plasmids, viral vectors without coverings, and even naked genes without any control region. The GDV may be either DNA or RNA, or may be a combination of the two, comprising both DNA and RNA in a single molecule.

Various viral vectors that can be used to introduce a nucleic acid sequence into the targeted patient's cells include, but are not limited to, vaccinia or other pox virus, herpes virus, retrovirus, or adenovirus. Techniques for incorporating DNA into such vectors are well known to those of ordinary skill in the art. Preferably, the retroviral vector is a derivative of a murine or avian retrovirus including, but not limited to, Moloney murine leukemia virus (MoMuLV), Harvey murine sarcoma virus (HaMuSV), murine mammary tumor virus (MuMTV), and Rous Sarcoma Virus (RSV). A retroviral vector may additionally transfer or incorporate a gene for a selectable marker (to aid in the identification or selection of transduced cells) or a gene that encodes the ligand for a receptor on a specific target cell (to render the vector target specific). For example, retroviral vectors can be made target specific by inserting a nucleotide sequence encoding a sugar, a glycolipid, or a protein. Targeting may also be accomplished using an antibody, by methods known to those of ordinary skill in the art.

Viral vectors are typically non-pathogenic (defective), replication competent viruses, which require assistance in order to produce infectious vector particles. This assistance can be provided, for example, by using helper cell lines that contain plasmids that encode all of the structural genes of the retrovirus under the control of regulatory sequences within the LTR, but that are missing a nucleotide sequence which enables the packaging mechanism to recognize an RNA transcript for encapsulation. Such helper cell lines include (but are not limited to) ψ2, PA317, and PA12. A retroviral vector introduced into such cells can be packaged and vector virion produced. The vector virions produced by this method can then be used to infect a tissue cell line, such as NIH 3T3 cells, to produce large quantities of chimeric retroviral virions.

GDV—Liposomes

Another delivery system, which can be targeted, for GPR 38 polynucleotides is a colloidal dispersion system. Colloidal dispersion systems include macromolecule complexes, nanocapsules, microspheres, beads, and lipid-based systems including oil-in-water emulsions, micelles, mixed micelles, and liposomes. One colloidal system for use as a delivery vehicle in vitro and in vivo is a liposome (e.g., an artificial membrane vesicle). Liposomes are small, lipid vesicles comprised of an aqueous compartment enclosed by a lipid bilayer, typically spherical or slightly elongated structures and several hundred angstroms in diameter. RNA, DNA, and intact virions can be encapsulated within the aqueous interior and be delivered to cells in a biologically active form, Fraley, et al., Trends iochem. Sci., 6:77 (1981).

Liposomes offer several readily exploited features. Under appropriate conditions, the liposome can fuse with the plasma membrane of a target cell or with the membrane of an endocytic vesicle within a cell which has internalized the liposome, thereby disgorging its contents into the cytoplasm. Prior to interaction with the surface of a target cell, however, the liposome membrane acts as a relatively impermeable barrier which sequesters and protects its contents, for example from degradative enzymes in the plasma. Liposomes have for this reason also been referred to as “micropills”. Additionally, because a liposome is a synthetic structure, custom-formulated liposomes can be designed that incorporate desirable features. Stryer, L., Biochemistry, 236-240 (1975) (W. H. Freeman, San Francisco); Szoka et al., Biochim. Biophys. Acta 600:1-18 (1980); Bayer et al., Biochim. Biophys. Acta. 550:464 (1979); Rivnay et al., Meth. Enzymol. 149:119 (1987); Wang et al., P.N.A.S. 84: 7851 (1987); and, Plant et al., Anal. Biochem. 176:420 (1989).

In addition to mammalian cells, including human cells, liposomes have been used for delivery of polynucleotides in plant, yeast, and bacterial cells. In order for a liposome to be an efficient gene transfer or delivery vehicle, the following characteristics should be present: (1) encapsulation of the genes of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information, Mannino, et al., Biotechniques, 6:882 (1988).

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticuloendothelial system (RES) in organs which contain sinusoidal capillaries. Active targeting, on the other hand, involves alteration of the liposome by coupling the liposome to a particular ligand such as a monoclonal antibody, sugar, glycolipid, or protein, or by changing the composition or size of the liposome in order to achieve targeting to organs and cell types other than the naturally occurring sites of localization.

GDV—Bacterial Cells

A bacterial cell suitable for use as a GDV within the present invention can be a bacterium that expresses a cytotoxic agent, such as an anti-tumor agent, on its cell surface or exported from the bacterium. Representative examples include BCG, Stover, Nature, 351:456-458 (1991) and Salmonella, Newton et al., Science 244:70-72 (1989). Eukaryotic cells suitable for use in the present invention include producer cells and ex vivo transduced cells.

GDV—Event Specific Promoters

Within some embodiments of the present invention, the GDV comprises a nucleic acid molecule under the transcriptional control of an event-specific promoter, such that upon activation of the event-specific promoter the nucleic acid molecule is expressed. Numerous event-specific promoters may be utilized within the context of the present invention, including for example, promoters that are activated by cellular proliferation (or are otherwise cell-cycle dependent) such as the thymidine kinase or thymidilate synthase promoters, Merrill, Proc. Natl. Acad. Sci. USA, 86:4987-91 (1989); Deng et al., Mol. Cell. Biol., 9:4079-82 (1989); promoters such as the α- or β-interferon promoters that are activated when a cell is infected by a virus, Fan and Maniatis, E.M.B.O. J., 8(1):101-110 (1989); Goodbourn et al. Cell, 45:601-610 (1986); and promoters that are activated by the presence of hormones, e.g., estrogen response promoters; see Toohey et al., Mol. Cell. Biol., 6:4526-38 (1986).

A recombinant viral vector (for example a recombinant MLV retrovirus) carries a gene expressed from an event-specific promoter, such as a cell cycle-dependent promoter (e.g., human cellular thymidine kinase or transferrin receptor promoters), which will be transcriptionally active primarily in proliferating cells, such as tumors. In this manner, replicating cells which contain factors capable of activating transcription from these promoters are preferentially affected (e.g., destroyed) by the agent produced by the GDV.

GDV—Tissue Specific Promoters

Within another embodiment of the present invention, the GDV comprises a nucleic acid molecule under the transcriptional control of a tissue-specific promoter, such that upon activation of the tissue-specific promoter the nucleic acid molecule is expressed. A wide variety of tissue-specific promoters may be utilized within the context of the present invention. Representative examples of such promoters include: liver-specific promoters such as Phospho-Enol-Pyruvate Carboxy-Kinase, Hatzogiou et al., J. Biol., Chem. 263: 17798-808 (1988); Benvenisty et al., Proc. Natl. Acad. Sci. USA, 86:1118-22 (1989); Vaulont et al., Mol. Cell. Biol., 9:4409-15 (1989), the albumin promoter and the alpha-fetoprotein (AFP) promoter, Feuerman et al., Mol. Cell. Biol., 9:4204-12 (1989); Camper and Tilghman, Genes Develop. 3:537-46 (1989); B cell specific promoters such as the IgG promoter; breast carcinoma or hepatocellular carcinoma specific promoters such as carcinoembryonic antigen (CEA) promoter, Schrewe et al., Mol. and Cell. Biol., 10:2738 (1990); pancreatic acinar cell specific promoters such as the elastase promoter, Swift et al., Genes Develop. 3:687-96 (1989); breast epithelial specific promoters such as the casein promoter, Doppler et al., Proc. Natl. Acad. Sci. USA, 86:104-08 (1989); erythroid specific-transcription promoters which are active in erythroid cells, such as the porphobilinogen deaminase promoter, Mignotte et al., Proc. Natl. Acad. Sci. USA, 86:6458-52 (1990); α- or β- globin specific promoters, van Assendelft et al., Cell, 56:969-77 (1989), Forrester et al., Proc. Natl. Acad. Sci. USA, 86:5439-43 (1989); promoters which regulate skeletal muscle such as the myo-D binding site, Burden, Nature, 341:716 (1989); Weintraub et al., Proc. Natl. Acad. Sci. USA, 86:5434-38 (1989); promoters which are specific for β cells of the pancreas, such as the insulin promoter, Ohlsson et al., Proc. Natl. Acad. Sci. USA, 85:4228-31 (1988); Karlsson et al., Mol. Cell. Biol., 9:823-27 (1989); promoters that are specific for the pituitary gland, such as the growth hormone factor promoter, Ingraham et al., Cell, 55:519-29 (1988); Bodner et al., Cell, 55:505-18 (1988); promoters which are specific for melanocytes, such as the tyrosine hydroxylase promoter; breast carcinoma specific promoters such as the HER2/neu promoter, Tal et al., Mol. Cell. Biol., 7:2597 (1987); liver-specific promoters such as the alcohol dehydrogenase (ADH) promoter, Felder, Proc. Natl. Acad. Sci. USA, 86:5903-07 (1989); T-cell specific promoters such as the T-cell receptor promoter, Anderson et al., Proc. Natl. Acad. Sci. USA, 85:3551-54 (1988); Winoto and Baltimore, E.M.B.O. J., 8:729-33 (1989); osteoblast or bone-specific promoters such as the osteocalcin promoter, Markose et al., Proc. Natl. Acad. Sci. USA, 87:1701-1705 (1990); McDonnell et al., Mol. Cell. Biol., 9:3517-23 (1989); Kemer et al., Proc. Natl. Acad. Sci. USA, 86:4455-59 (1989) the IL-2 promoter, IL-2 receptor promoter, the whey (WAP) promoter, and the MHC Class II promoter.

GDV—Tissue and Event Specific Promoters

The GDV can also comprise a nucleic acid molecule under the transcriptional control of both an event-specific promoter and a tissue-specific promoter, such that the nucleic acid molecule is maximally expressed only upon activation of both the event-specific promoter and the tissue-specific promoter. In particular, by utilizing such vectors, the substance expressed from the nucleic acid molecule is expressed only in cell types satisfying both criteria (e.g., in the example above, combined promoter elements are functional only in rapidly dividing liver cells). Within preferred embodiments of the invention, the number of transcriptional promoter elements may also be increased, in order to improve the stringency of cell-type specificity.

GDV—Other Specific Control Elements

A variety of other elements that control gene expression may also be utilized within the context of the present invention, including for example locus-defining elements such as the β-globin gene and the T cell marker CD2. In addition, elements which control expression at the level of splicing and nuclear export are the β-globin intron sequences, the rev and rre elements in HIV-1, and the CTE element in the D-type masonpfizer monkey retrovirus.

GDV—Cancer Directed Vector Systems

Within preferred embodiments of the invention, the GDV is a retroviral vector and the gene produces an agent against a tumor, the gene being under control of a tissue-specific promoter having specificity for the tissue of tumor origin. Since the retroviral vector preferentially integrates into the genome of replicating cells (for example, normal liver cells are only slowly replicating, while those of a hepatocarcinoma are replicating more quickly), these two levels of specificity (viral integration/replication and tissue-specific transcriptional regulation) lead to preferential killing of tumor cells.

Transcriptional promoter/enhancer elements as discussed above need not necessarily be present as an internal promoter (lying between the viral LTRs for retroviruses, for example), but may be added to or replace the transcriptional control elements in the viral LTRs which are themselves transcriptional promoters, such that condition-specific (e.g., event or tissue specific) transcriptional expression will occur directly from the modified viral LTR. In this case, either the condition for maximal expression will need to be mimicked in retroviral packaging cell lines (e.g., by altering growth conditions, supplying necessary transregulators of expression or using the appropriate cell line as a parent for a packaging line), or the LTR modification is limited to the 3′ LTR U3 region, to obtain maximal recombinant viral titers. In the latter case, after one round of infection/integration, the 3′ LTR U3 is now also the 5′ LTR U3, giving the desired tissue-specific expression. Similarly, for other viral vectors, the promoters may be exogenous, or hybrids with normal viral promoter elements.

GDV—Eukaryotic Layered Systems

The present invention also provides eukaryotic layered vector initiation systems, which are generally comprised of a 5′ promoter, a construct that is capable of expressing one or more heterologous nucleotide sequences, and, of replication in a cell either autonomously or in response to one or more factors, a polyadenylation sequence, and a transcription termination sequence. Briefly, eukaryotic layered vector initiation systems provide a two stage or “layered” mechanism that controls expression of heterologous nucleotide sequences. The first layer initiates transcription of the second layer, and comprises a 5′ promoter, polyadenylation site, and transcription termination site, as well as one or more splice sites if desired. Representative examples of promoters suitable for use in this regard include any viral or cellular promoters such as CMV, retroviral LTRs, SV40, β-actin, immunoglobulin promoters, and inducible promoters such as the metallothionein promoter and glucocorticoid promoter. The second layer comprises a construct which is capable of expressing one or more heterologous nucleotide sequences, and, of replication in a cell either autonomously or in response to one or more factors. Within one embodiment of the invention the construct may be a Sindbis GDV as described above.

The GDV in this and other embodiments can include one or both of a marker gene, such as neomycin resistance, and a “suicide gene,” such as the herpes simplex virus thymidine kinase (HSVTK) gene.

The GDV is then introduced into suitable packaging cell lines, which cell lines can be selected for particularly desirable characteristics, such as where the GDVs each display amphotropic, xenotropic or polytropic characteristics. Other suitable packaging cell lines include the 293 2-3 VSV-G system, and cell lines that exhibit vector structural protein modified to facilitate targeting of the transduction of the vector to a preferred location (e.g., a regional lymph node or a cell that presents a particular antigen). The cell lines can then be tested to confirm that they contain the desirable components.

Next, cell cultures are prepared, and supernatant fluids that contain the retroviral vectors are harvested. The fluids can be tested for GDV potency, typically measured in colony forming units (CFU) or plaque forming units (PFU), as appropriate. In one approach, the GDV themselves are not further processed prior to administration to the host animal or plant. In a preferred approach, the GDV is then concentrated, purified, and formulated before administration.

### EXAMPLES

The Examples below provide information as follows: Example 1 relates to the identification and selection of appropriate antigens for IHC analyses. Examples 2 to 4 relate to antibody production and purification based on such antigens. Examples 5 to 10 relate to H&E staining. Example 11 relates to Western blot analyses, and Example 12 relates to results from such analyses.

### Example 1

### Selection of Antigens

Antigenic peptides were derived from the amino acid sequence of GPR 38 based on analyses of likely antigen-containing regions. Design of antigen peptides (approximately 20 amino acids in length) for antibody generation was performed using basic techniques, including BLAST methods of peptide analysis to determine regions comprising (1) specificity to the protein/gene of interest, and (2) antigenicity. With respect to specificity, parameters that precluded the use of a particular peptide included the presence of 6 or more contiguous amino acids with sequence identity to protein(s) other than the protein of interest, the presence of sites of posttranslational modification, including phosphorylation and glycosylation, and highly hydrophobic sequences, which could indicate potential in situ localization within the plasma membrane. The selected antigens were as follows: REPPWPALPPCDERRCS, SEQ ID NO: 3, SPPSGPETAEAAALFSREC, SEQ ID NO: 4, SSRRPLRGPAASGRERGHRQ, SEQ ID NO: 5, and RKSRPRGFHRSRDTAG, SEQ ID NO: 6.

### Example 2

### Antibody Production Schedule

Day 0—Pre-immune serum collection (approximately 5.0 ml). Immunize using 200 μg antigen peptide per rabbit in Complete Freund's Adjuvant.

Day 14—Immunize using 100 μg antigen per rabbit in Incomplete Freund's Adjuvant.

Day 28—Immunize using 100 μg antigen per rabbit in Incomplete Freund's Adjuvant.

Day 42—Immunize using 100 μg antigen per rabbit in Incomplete Freund's Adjuvant.

Day 49—First production bleed; obtain 24.0-26.0 ml.

Day 56—Immunize using 100 μg antigen per rabbit in Incomplete Freund's Adjuvant.

Day 63—Second production bleed and ELISA analysis.

Day 70—Immunize using 100 μg antigen per rabbit in Incomplete Freund's Adjuvant.

Day 77—Third production bleed and affinity purification.

### Example 3

### Immunosorbent Purification of Antiserum Coupling of Peptide to CNBr-Activated Sepharose 4B

Weigh out 0.8 g of CNBr-activated Sepharose 4B (2.5 ml of final gel volume). Wash and re-swell on sintered glass filter with 1 mM HCl, followed by coupling buffer (0.1 M NaHCO₃, 0.25 M NaCl, pH 8.5). Dissolve 10 mg of protein or peptide in coupling buffer. Mix protein solution with gel suspension and incubate 2 hours at room temperature or overnight at 4° C. Block remaining active groups with 0.2 M glycine buffer, pH 8.1. Wash away excess adsorbed protein with coupling buffer, followed by 0.1 M acetate buffer containing 0.5 M NaCl, pH 4.3. Equilibrate the column with phosphate-buffered saline (PBS), pH 7.7.

### Example 4

### Immunosorbent Purification of Antiserum Affinity Purification of Antiserum

Dilute 10 ml of clear antiserum 1:1 with PBS, pH 7.7, apply to affinity column at a flow rate of 0.3 ml/minute, and monitor absorbance of eluate at 280 nm. Collect fractions of unbound material and rinse column with PBS, pH 7.7. Elute bound antibody with 0.2 M glycine, pH 1.85, and collect eluate until absorbance at 280 nm returns to baseline. Neutralize all collected fractions with 1 M Tris-HCl, pH 8.5 immediately after collection. Determine OD at 280 nm, and determine the total OD recovered. Conduct ELISA analysis with the corresponding antigen to confirm the presence and identity of recovered antibody and the removal of all antibody from the original serum. Concentrate antibody to approximately 2.0 mg/ml and dialyze against PBS with 0.01% NaN₃.

### Example 5

### Preparation of Antibody Dilutions

The purpose of this protocol was to dilute antibodies in solution. Materials include Tris-HCL Buffer with carrier protein and 0.015 M NaN₃ (Dako Antibody Diluent #S0809 (DAKO, Carpentaria, Calif.); vials containing the antibodies described above or commercial antibodies against GPR 38; pipetmen and disposable tips; container of chopped ice; 12 ml Dako reagent tubes; and, reagent tube rack.

The procedure was a) calculate proportions of antibody and diluent according to desired concentrations and volume requirements; b) label reagent tubes and place in rack; c) pipette needed volume of diluent into tube(s); d) place vials of antibodies into ice; e) invert and/or flick antibody vial(s) 3 or 4 times to insure suspension; f) pipette required volume of antibody(s) into corresponding diluent volumes; and, g) mix gently.

### Example 6

### Preparation of Autostainer Solutions

The purpose of this protocol was the preparation of concentrated solutions for use in a DAKO autostainer. Materials include DAKO® TBST (Tris Buffered Saline Containing Tween-S3306), 10× Concentrate, DAKO® Target Retrieval Solution, 10× Concentrate (S1699), deionized H₂O, 20L container, with lid, marked at the 10L level, DAKO® TBS (Tris Buffered Saline-S1968), and DAKO Tween® (S1966).

The procedure to make TBST 10× Concentrate was a) pour 2 500 ml bottles DAKO® TBST into a 20 L container, b) add deionized H₂O until solution level was at 10 L mark, c) replace lid and shake 10 to 20 times, d) pour diluted DAKO® TBST into autostainer carboy(s) as designated. The procedure to make Target Retrieval Solution was a) measure 135 ml of deionized H₂O and pour into slide bath, b) measure 15 ml of DAKO® Target Retrieval solution, c) add to H₂O, and d) agitate. This solution was then used in the steam method of target retrieval, Example 9, below. The procedure to make TBS was a) fill 20L container to 10L mark with deionized H₂O, b) add 2 envelopes of DAKO® TBS, c) add 5 ml of DAKO TWEEN®, and d) replace lid and agitate 10 to 20 times.

### Example 7

### Preparation of Solutions for Antibody Detection

Solutions for antibody detection were prepared using Vector® Biotinylated antibody (BA series), Vectastain® ABC-AP Kit (AK-5000), 10 mM sodium phosphate, pH 7.5, 0.9% saline (PBS), Vector® Red Alkaline Phosphatase Substrate Kit I (SK-5100), and 100 mM Tris-HCl, pH 8.2 Buffer. To prepare biotinylated antibody, add 10 ml of PBS to reagent tube, add 1 drop biotinylated antibody to the PBS, then mix gently. To prepare ABC, to 10 ml of PBS, add 2 drops each of Reagent A and Reagent B, mix immediately, then allow to stand 30 minutes before use. To prepare AP Red, which should be prepared immediately before use, to 5 ml of Tris-HCl buffer, add 2 drops of Reagent 1 and mix well, add 2 drops of Reagent 2 and mix well, then add 2 drops of Reagent 3 and mix well.

### Example 8

### Deparaffinization and Rehydration of Samples

The purpose of this protocol was to remove paraffin from and rehydrate preserved tissues in preparation for IHC procedures. Materials and equipment include fume hood, vertical slide rack(s), three xylene (VWR #72060-088) baths, three 100% alcohol blend (VWR #72060-050) baths, two 95% alcohol blend (VWR #72060-052) baths, one 70% alcohol blend (VWR #72060-056) bath, and Tris-Buffered Saline (DAKO® S1968)+Tween® (DAKO S1966).

Insert the slides into the vertical rack(s). Move slides through baths inside fume hood as follows:

Xylene 5 Minutes

Xylene 5 Minutes

Xylene 5 Minutes

100% Alcohol 2 Minutes

100% Alcohol 2 Minutes

100% Alcohol 1 Minute

95% Alcohol 2 Minutes

95% Alcohol 2 Minutes

70% Alcohol 1 Minute

Finally, place slides into a container with TBST.

### Example 9

### Steam Method of Target Retrieval

The purpose of this protocol was to optimize antibody binding within paraffin embedded tissues. Materials and equipment included a steamer, deionized H₂O, target retrieval solution, 10× concentrate (DAKO #S1699), 250 ml graduated cylinder, 15 ml graduated cylinder, staining dish(es), and deparaffinized and rehydrated tissue on microscope slides in immersed TBST. The procedure was to a) fill the steamer with deionized H₂O to appropriate depth as indicated, b) turn the steamer on, c) in a graduated cylinder, measure 135 ml of deionized H₂O and pour into staining dish(es), d) pipette 15 ml of target retrieval solution and release into deionized H₂O, e) place the staining dish(es) into the basket of the steamer and heat for at least 10 minutes to preheat, f) add rack(s) containing tissue slides to heated target retrieval solution, g) cover and steam for 20 minutes, h) remove container from steamer and let stand at room temperature for 20 minutes, i) transfer rack(s) with slides to container(s) of TBST, and j) slides are now ready for staining procedures.

### Example 10

### Antibody Detection

The deparaffinized, rehydrated, and steamed (if needed) slides were loaded onto racks within a DAKO autostainer and then the autostainer was run according to the manufacturer's instructions. The slides were removed and the autostainer was turned off.

### Example 11

### Western Blotting

The purpose of this protocol was to visualize the immunoreactivity of the antibodies described above against GPR 38 on a western blot. Materials and equipment included western blot membrane, TBS Tween (TBST: 100 mM Tris-HCl pH 7.5, 150 mM NaCl, 0.1% Tween™ 20), 5% non-fat dried milk in TBST (blotto), antibody of interest (primary), peroxidase-conjugated AffiniPure goat anti-rabbit IgG (H+L) (secondary)-Jackson ImmunoResearch, ECL solution (Amersham Biosciences, Uppsala Sweden), film, developer D-19, fixer, rocking platform.

During the blotting procedure, the blot was kept wet at all times and on a substantially level surface. The Western blot was placed right-side up in 10 ml of blotto. The membrane was flipped over and the dish rocked so that the solution covered it. The membrane was then flipped back to the right side and solution was again rocked over it. The blot was then placed on a shaker for at least 1 hour. Ten ml of primary antibody were prepared by diluting 1:500 in blotto.

The blotto was removed from the Western blot and replaced with the primary antibody. The blot was flipped again and placed on the shaker for 1 hour. Secondary antibody and peroxidase-conjugated AffiniPure goat anti-rabbit IgG (H+L) are prepared 1:20,000 in 10 ml of blotto. The primary antibody was removed and the Western blot was washed 3 times with 10 ml of blotto. The blotto was removed and replaced with the secondary antibody solution. The blot was flipped and placed on the shaker for 1 hour. The secondary antibody was removed and the blot washed 2 times with 10 ml of blotto. The blotto was removed and the blot was washed 2 times with 10 ml TBST. ECL was prepared by combining equal amounts of Solution 1 and 2.

The blotto was removed and 1 ml of ECL was placed on the blot. The blot was flipped and let sit for 1 minute. The blot was placed on plastic wrap and immediately covered with plastic wrap. The ECL was pressed out. The blot was placed on the film, then the film was developed.

### Example 12

### Results

A summary of the results of these analyses are reported in the text above, for example in the Expression Profile of GPR 38 portion of the General Discussion of Nucleic Acids and Polypeptides Related to GPR 38. SPPSGPETAEAAALFSREC, SEQ ID NO: 4, SSRRPLRGPAASGRERGHRQ, SEQ ID NO: 5 and a pool of all of the antigens set forth in SEQ ID NOS: 3-6 were each used to create antibodies as described above for the analyses.

The following comparison statements are arranged as follows within each disease section:

Analyses based on antibodies against SPPSGPETAEAAALFSREC, SEQ ID NO: 4.

Analyses based on antibodies against SSRRPLRGPAASGRERGHRQ, SEQ ID NO: 5.

Analyses based on antibodies against the pooled antigens set forth in SEQ ID NOS: 3-6.

Brain, Alzheimer's Disease (cortex):

Compared to normal cortex, samples of Alzheimer's disease showed reduced staining of neurons and astrocytes, and focal intense staining within plaques.

Compared to normal cerebral cortex, samples of Alzheimer's disease showed reduced staining of neurons and astrocytes, and senile plaques had faint staining surrounding negative amyloid cores.

Compared to normal cortex samples of Alzheimer's disease showed reduced staining of neurons and astrocytes.

Brain, Infarct (cerebral cortex and caudate):

Compared to normal cortex and caudate, samples of brain infarct showed reduced staining of neurons and astrocytes and faint staining of reactive endothelial cells.

Compared to normal cerebral cortex and caudate, samples of brain infarct showed no significant changes.

Compared to normal cortex and caudate, samples of brain infarct showed no significant differences.

Brain, Glioblastoma:

Compared to normal cortex, samples of glioblastoma showed focally strongly positive neoplastic cells.

Compared to normal cortex, samples of glioblastoma showed positive neoplastic glial cells.

Compared to normal cortex, samples of glioblastoma showed no significant differences.

Brain, Parkinson's Disease (substantia nigra):

Pigmented neurons were negative for staining. Nonpigmented neurons were negative. Neuropil was negative. The level of staining in pigmented neurons appeared less than that in the normal substantia nigra. Lewy bodies were negative for staining.

Pigmented neurons were negative for staining. Nonpigmented neurons were negative. Neuropil was negative. The level of staining in both pigmented and nonpigmented neurons appeared less than that in the normal substantia nigra. Lewy bodies were negative for staining.

Pigmented neurons were negative for staining. Nonpigmented neurons were predominantly negative, rarely faintly positive. Neuropil was negative to faintly positive. The level of staining in pigmented and nonpigmented neurons appeared less than that in the normal substantia nigra. Lewy bodies were negative for staining.

Colon, Ulcerative Colitis:

Compared to normal colon, samples of ulcerative colitis showed loss of staining of the superficial epithelium and APUD cells.

Compared to normal colon, samples of ulcerative colitis showed similar features.

ompared to normal colon, samples of ulcerative colitis showed loss of staining of APUD cells and ganglion cells of the enteric plexus, and staining of some reactive fibroblasts and transforming lymphoid cells.

Heart, Diabetes:

There was no significant difference in the level of staining in myocytes overall in diabetes compared to normal heart samples.

There was increased staining in myocytes in one sample of diabetes compared to normal heart samples.

There was no significant difference in the level of staining in myocytes in diabetes compared to normal heart samples.

Heart, Heart Failure:

There was no significant difference in the level of staining in myocytes in heart failure compared to normal heart samples.

There was no significant difference in the level of staining in myocytes in heart failure compared to normal heart samples.

Cardiac myocytes were negative except for scattered moderately positive fibers in one sample more apparent within the subendocardial region. This sample showed a significant difference in the level of staining in myocytes in heart failure compared to normal heart samples.

Heart, Myocardial Infarct:

Compared to normal heart samples, there was no significant difference in staining in acutely injured myocytes.

Compared to normal heart samples, there was increased staining in acutely injured myocytes.

Compared to normal heart samples, there no significant difference in staining in acutely injured myocytes.

Kidney, Diabetes:

Compared to normal kidney samples, samples of diabetic kidney showed no significant differences.

Compared to normal kidney samples, samples of diabetic kidney showed similar features.

Kidney, Hypertension:

Compared to normal kidney samples, samples of kidney from hypertensive patients showed no significant differences.

Compared to normal kidney samples, samples of kidney from hypertensive patients showed distinctive proximal tubular staining and interstitial fibroblast staining.

Compared to normal kidney samples, samples of kidney from hypertensive patients showed focally increased staining of proximal and distal tubules, and reduced overall staining in one sample that showed atrophy.

Lung, Asthma:

Compared to normal lung samples, lung from asthmatic patients showed no significant differences.

Compared to normal lung samples, lung from asthmatic patients showed reduced staining of Type II pneumocytes and alveolar macrophages.

Compared to normal lung samples, lung from asthmatic patients showed no significant differences.

Lung, Bronchitis:

Compared to normal lung samples, lung from patients with bronchitis showed no significant differences.

Compared to normal lung samples, lung from patients with bronchitis showed reduced staining of respiratory epithelium, Type II pneumocytes and alveolar macrophages.

Compared to normal lung samples, lung from patients with bronchitis showed no significant differences.

Lung, Emphysema:

Compared to normal lung samples, lung from patients with emphysema showed no significant differences.

Compared to normal lung samples, lung from patients with emphysema showed reduced staining of bronchial epithelium and alveolar septa, as well as reduced staining of alveolar macrophages.

Compared to normal lung samples, lung from patients with emphysema showed no significant differences.

Lung, Pneumonia:

Compared to normal lung samples, lung from patients with pneumonia showed loss of staining of Type II pneumocytes and alveolar macrophages.

Compared to normal lung samples, lung from patients with pneumonia showed no significant changes.

Lymph Node, Hodgkin's Lymphoma:

Compared to normal lymph node, samples from patients with Hodgkin's lymphoma showed positivity of Reed Sternberg cells.

Compared to normal lymph node, samples from patients with Hodgkin's lymphoma showed positive Reed Sternberg cells and eosinophils.

Compared to normal lymph node, samples from patients with Hodgkin's lymphoma showed faintly positive Reed Sternberg cells and punctate cytoplasmic and/or nuclear positivity of a subpopulation of lymphoid cells.

Lymph Node, Non-Hodgkin's Lymphoma:

Compared to normal lymph node, samples from patients with Non-Hodgkin's lymphoma showed no significant differences.

Compared to normal lymph node, samples from patients with Non-Hodgkin's lymphoma showed focal staining of neoplastic lymphoid cells.

Compared to normal lymph node, samples from patients with Non-Hodgkin's lymphoma showed no significant differences.

Prostate, Benign Prostatic Hyperplasia:

Compared to normal prostate, samples of benign prostatic hyperplasia showed increased staining of prostate epithelium, notably basal cells.

Compared to normal prostate, samples of benign prostatic hyperplasia showed increased staining of epithelium.

Compared to normal prostate, samples of benign prostatic hyperplasia showed no significant differences.

Small Intestine, Crohn's Disease:

Compared to normal samples, samples of small intestine from patients with Crohn's disease showed reduced staining of crypt epithelium and APUD cells.

Compared to normal samples, samples of small intestine from patients with Crohn's disease showed faintly positive cosinophils.

Compared to normal samples, samples of small intestine from patients with Crohn's disease showed loss of staining of APUD cells and of Auerbach's plexus.

From the foregoing, it will be appreciated that, although specific embodiments of the invention have been described herein for purposes of illustration, various modifications may be made without deviating from the spirit and scope of the invention. Accordingly, the invention includes all permutations and combinations of the subject matter set forth herein and is not limited except as by the appended claims.

What is claimed is:

1. An assay for the detection of an increased possibility of Alzheimer's disease in a human patient, comprising:
a) providing a binding partner specific for GPR 38,
b) contacting the binding partner with at least one of neurons and astrocytes of the patient under conditions suitable and for a time sufficient for the binding partner to bind to GPR 38 in the at least one of the neurons and astrocytes,
c) detecting the binding partner bound to the GPR 38,
d) determining whether the at least one of the neurons and astrocytes contain reduced levels of GPR 38 relative to normal and therefrom determining whether the patient has an increased possibility of Alzheimer's disease.

2. The assay of claim 1 wherein the binding partner is an antibody.

3. The assay of claim 1 or 2 wherein the neurons and astrocytes are in at least one biopsy removed from a living patient.

4. The assay of claim 1 or 2 wherein the neurons and astrocytes are in at least one tissue sample removed from a deceased patient.

5. An assay for the detection of an increased possibility of Parkinson's disease in a human patient, comprising:
a) providing a binding partner specific for GPR 38,
b) contacting the binding partner with at least one of neurons and neuropil from a substantia nigra of the patient under conditions suitable and for a time sufficient for the binding partner to bind to GPR 38 in the at least one of the neurons and neuropil,
c) detecting the binding partner bound to the GPR 38,
d) determining whether the at least one of the neurons and neuropil contain decreased levels of GPR 38 relative to normal and therefrom determining whether the patient has an increased possibility of Parkinson's disease.

6. The assay of claim 5 wherein the binding partner is an antibody.

7. The assay of claim 5 or 6 wherein the neurons and neuropil are in at least one biopsy removed from a living patient.

8. The assay of claim 5 or 6 wherein the neurons and neuropil are in at least one tissue sample removed from a deceased patient.

9. An assay for the detection of an increased possibility of ulcerative colitis in a human patient, comprising:
a) providing a binding partner specific for GPR 38,
b) contacting the binding partner with at least one of surface epithelium, neuroendocrine cells, enteric plexus ganglion cells, subsets of lymphoid cells, and subsets of fibroblasts from a colon of the patient under conditions suitable and for a time sufficient for the binding partner to bind to GPR 38 in the at least one of the surface epithelium, neuroendocrine cells, enteric plexus ganglion cells, subsets of lymphoid cells, and subsets of fibroblasts,
c) detecting the binding partner bound to the GPR 38,
d) determining whether the at least one of the surface epithelium, neuroendocrine cells, and the enteric plexus ganglion cells contain reduced levels of GPR 38 relative to normal or whether the at least one of the subsets of lymphoid cells and the subsets of fibroblasts contain increased levels of GPR 38 relative to normal, and therefrom determining whether the patient has an increased possibility of ulcerative colitis.

10. The assay of claim 9 wherein the binding partner is an antibody.

11. The assay of claim 9 or 10 wherein the at least one of the surface epithelium, neuroendocrine cells, enteric plexus ganglion cells, lymphoid cell,s and fibroblasts are in at least one biopsy removed from a living patient.

12. The assay of claim 9 or 10 wherein the at least one of the surface epithelium, neuroendocrine cells, enteric plexus ganglion cells, subsets of lymphoid cells, and subsets of fibroblasts are in at least one tissue sample removed from a deceased patient.

13. An assay for the detection of an increased possibility of Crohn's disease in a human patient, comprising:
a) providing a binding partner specific for GPR 38,
b) contacting the binding partner with at least one of absorptive epithelium, neuroendocrine cells, and eosinophils from a small intestine of the patient under conditions suitable and for a time sufficient for the binding partner to bind to GPR 38 in the at least one of the absorptive epithelium, neuroendocrine cells, and eosinophils,
c) detecting the binding partner bound to the GPR 38,
d) determining whether the at least one of the absorptive epithelium and neuroendocrine cells contain reduced levels of GPR 38 relative to normal or whether the eosinophils contain increased levels of GPR 38 relative to normal, and therefrom determining whether the patient has an increased possibility of Crohn's disease.

14. The assay of claim 13 wherein the binding partner is an antibody.

15. The assay of claim 13 or 14 wherein the at least one of the absorptive epithelium, neuroendocrine cells, and eosinophils are in at least one biopsy removed from a living patient.

16. The assay of claim 13 or 14 wherein the at least one of the absorptive epithelium, neuroendocrine cells, and eosinophils are in at least one tissue sample removed from a deceased patient.

17. An assay for the detection of an increased possibility of Hodgkin's disease in a human patient, comprising:
a) providing a binding partner specific for GPR 38,
b) contacting the binding partner with Reed Sternberg cells and reactive lymphoid cells from the patient under conditions suitable and for a time sufficient for the binding partner to bind to GPR 38 in the Reed Sternberg cells and reactive lymphoid cells,
c) detecting the binding partner bound to the GPR 38,
d) determining whether the Reed Sternberg cells contain increased levels of GPR 38 relative to normal and the reactive lymphoid cells contain focal punctuate staining of GPR 38, and therefrom determining whether the patient has an increased possibility of Hodgkin's disease.

18. The assay of claim 17 wherein the binding partner is an antibody.

19. The assay of claim 17 or 18 wherein the Reed Sternberg cells and reactive lymphoid cells are in at least one biopsy removed from a living patient.

20. The assay of claim 17 or 18 wherein the Reed Sternberg cells and reactive lymphoid cells are in at least one tissue sample removed from a deceased patient.

21. An assay for the detection of an increased possibility of glioblastoma in a human patient, comprising:
a) providing a binding partner specific for GPR 38,
b) contacting the binding partner with neoplastic glial cells from the patient under conditions suitable and for a time sufficient for the binding partner to bind to GPR 38 in the neoplastic glial cells and reactive lymphoid cells,
c) detecting the binding partner bound to the GPR 38,
d) determining whether the neoplastic glial cells contain increased levels of GPR 38 relative to normal and therefrom determining whether the patient has an increased possibility of glioblastoma.

22. The assay of claim 21 wherein the binding partner is an antibody.

23. The assay of claim 21 or 22 wherein the neoplastic glial are in a biopsy removed from a living patient.

24. The assay of claim 21 or 22 wherein the neoplastic glial cells are in a tissue sample removed from a deceased patient.

25. An assay for the detection of an increased possibility of carcinoma selected from the group consisting of breast carcinoma, colon carcinoma, lung small cell carcinoma, lung adenocarcinoma, ovarian carcinoma, pancreatic small cell carcinoma, pancreatic adenocarcinoma and prostate carcinoma in a human patient, comprising:
a) providing a binding partner specific for GPR 38,
b) contacting the binding partner with cells from a tissue selected from the group consisting of breast, colon, lung, ovarian, pancreas and prostate from the patient under conditions suitable and for a time sufficient for the binding partner to bind to GPR 38 in the tissue from the group consisting of breast, colon, lung, ovarian, pancreas and prostate,
c) detecting the binding partner bound to the GPR 38,
d) determining whether the tissue from the group consisting of breast, colon, lung, ovarian, pancreas and prostate contain increased levels of GPR 38 relative to normal and therefrom determining whether the patient has an increased possibility of carcinoma selected from the group consisting of breast carcinoma, colon carcinoma, lung small cell carcinoma, lung adenocarcinoma, ovarian carcinoma, pancreatic small cell carcinoma, pancreatic adenocarcinoma and prostate carcinoma wherein the tissue selected corresponds to the tissue potentially containing the possible carcinoma.

26. The assay of claim 25 wherein the binding partner is an antibody.

27. The assay of claim 25 or 26 wherein the tissue from the group consisting of breast, colon, lung, ovarian, and pancreas is in a biopsy removed from a living patient.

28. The assay of claim 25 or 26 wherein the tissue from the group consisting of breast, colon, lung, ovarian, and pancreas is in a tissue sample removed from a deceased patient.

29. The assay of claim 25 or 26 wherein the tissue is breast and the carcinoma is breast carcinoma.

30. The assay of claim 25 or 26 wherein the tissue is colon and the carcinoma is colon carcinoma.

31. The assay of claim 25 or 26 wherein the tissue is lung and the carcinoma is lung small cell carcinoma.

32. The assay of claim 25 or 26 wherein the tissue is lung and the carcinoma is lung adenocarcinoma.

33. The assay of claim 25 or 26 wherein the tissue is ovarian and the carcinoma is ovarian carcinoma.

34. The assay of claim 25 or 26 wherein the tissue is pancreas and the carcinoma is pancreatic small cell carcinoma.

35. The assay of claim 25 or 26 wherein the tissue is pancreas and the carcinoma is pancreatic adenocarcinoma.

36. The assay of claim 25 or 26 wherein the tissue is prostate and the carcinoma is prostate carcinoma.

37. A kit for the detection of antibodies against GPR 38 for use in an assay according to any one of claims 1, 2, 5, 6, 9, 10, 13, 14, 17, 18, 21, 22, 25 or 26, the kit comprising:
a) an antibody specific for GPR 38,
b) one or both of a reagent or a device for detecting the antibody, and
c) a label stating that the kit is to be used in the assay.

38. The kit of claim 37 where in the label is an FDA approved label.

39. An isolated and purified composition comprising GPR 38 and a pharmaceutically acceptable carrier for use in the manufacture of a medicament for inhibiting, preventing or treating at least one of Alzheimer's disease, Parkinson's disease, ulcerative colitis, Crohn's disease, Hodgkin's disease, glioblastoma, breast carcinoma, colon carcinoma, lung small cell carcinoma, lung adenocarcinoma, pancreatic small cell carcinoma and pancreatic adenocarcinoma.

40. A method of manufacturing a medicament able to reduce symptoms associated with Alzheimer's disease, Parkinson's disease, ulcerative colitis, Crohn's disease, Hodgkin's disease, glioblastoma, breast carcinoma, colon carcinoma, lung small cell carcinoma, lung adenocarcinoma, pancreatic small cell carcinoma and pancreatic adenocarcinoma in a human patient, comprising combining a pharmaceutically effective amount of a GPR 38 agonist, a pharmaceutically acceptable carrier, adjuvant, excipient, buffer and diluent.

41. A method of manufacturing a medicament able to reduce symptoms associated with Alzheimer's disease, Parkinson's disease, ulcerative colitis, Crohn's disease, Hodgkin's disease, glioblastoma, breast carcinoma, colon carcinoma, lung small cell carcinoma, lung adenocarcinoma, pancreatic small cell carcinoma and pancreatic adenocarcinoma in a human patient, comprising combining a pharmaceutically effective amount of a GPR 38 antagonist, a pharmaceutically acceptable carrier, adjuvant, excipient, buffer and diluent
